(19) United States
(12) Patent Application Publication
(10) Pub. No.: US 2026/0263091 A1
(43) Pub. Date: Sep. 10, 2026

(54) Apparatus and Methods for Clot Aspiration With Revolving Asymmetric Aspiration Zone

(71) Applicant: Dragon Vascular, Inc., Fremont, CA (US)

(72) Inventors: Jayson Delos Santos, Alamo, CA (US); Michael P. Wallace, Pleasanton, CA (US); Roy Leguidleguid, Fremont, CA (US); Adam Tanner, Campbell, CA (US); Erik T. Engelson, Menlo Park, CA (US)

(73) Assignee: Dragon Vascular, Inc., Fremont, CA (US)

(21) Appl. No.: 19/558,930

(22) Filed: Mar. 6, 2026

Related U.S. Application Data
(63) Continuation of application No. PCT/US2026/017719, filed on Mar. 4, 2026.
(60) Provisional application No. 63/766,947, filed on Mar. 4, 2025.
(60) Provisional application No. 63/773,429, filed on Mar. 17, 2025.
(60) Provisional application No. 63/800,430, filed on May 5, 2025.
(60) Provisional application No. 63/854,449, filed on Jul. 30, 2025.
(60) Provisional application No. 63/932,601, filed on Dec. 5, 2025.

(51) Int. Cl. A61B 17/22; A61B 17/00; A61M 25/00

(52) U.S. Cl. CPC A61B 17/22; A61B 2017/00292; A61B 2017/00398; A61B 2017/22079; A61M 2025/0046; A61M 25/0097; A61M 2206/20

(57) ABSTRACT

A system for aspirating clot from a blood vessel includes an aspiration catheter and a clot-disruption structure. The catheter includes a body, a clot-extraction lumen with an open distal region, and a central axis. A clot-disruption structure such as a disruptor revolves in and/or across the open distal region of the clot-extraction lumen and disrupts the clot to facilitate aspiration into and through the clot-extraction lumen with reduced risk of clogging. The clot-disruption element may comprise a solid disc. A drive cable is asymmetrically attached to a proximal portion of the clot-disruption element and rotates about its own axis to cause the clot-disruption structure to both revolve and rotate in the open distal region of the aspiration catheter.

### CROSS-REFERENCES TO RELATED APPLICATIONS

The present application is a Continuation of PCT/US2026/017719 filed Mar. 4, 2026; which claims the benefit of U.S. Provisional Patent Appln. Nos. 63/766,947 filed Mar. 4, 2025; 63/773,429 filed Mar. 17, 2025; 63/800,430 filed May 5, 2025; 63/854,449 filed Jul. 30, 2025; and 63/932,601 filed Dec. 5, 2025; the full disclosures which are incorporated herein by reference in their entirety for all purposes.

### BACKGROUND

### Field

The disclosed technology relates generally to medical devices and methods. More particularly, the disclosed technology relates to apparatus and methods for vascular clot removal.

Ischemic strokes are a leading cause of death and disability worldwide and result from blockage or interruption of blood flow within a cerebral artery, typically the middle cerebral artery. The blockage of blood flow can damage brain tissue, which in some cases may be treatable but which is often irreversible if the blockage is not removed within a short period of time.

Small vessel ischemic stroke may be treated by delivering thrombolytic drugs, such as tPA (tissue plasminogen activator), but large vessel ischemic stroke often presents a high clot burden which cannot be treated with thrombolytic drugs alone. In such cases, mechanical thrombectomy has become the standard of care.

Mechanical thrombectomy is a minimally invasive intervention where a catheter is intravascularly advanced into the patient's blood vessel to remove the blood clot and restore blood flow to the affected tissue. One common type of mechanical thrombectomy is referred to as “aspiration thrombectomy” where a negative pressure is applied through a clot-extraction lumen of the catheter to draw clot from blood vessels. A second type of mechanical thrombectomy uses a mechanical element to dislodge and remove the clot. For example, a stent-like or corkscrew-like retriever element may be extended beyond the catheter tip into the blood vessel and used to pull clot from the blood vessel. Aspiration is sometimes, but not always, combined with the use of mechanical retrieval elements.

Although an effective treatment in many cases, presently available mechanical thrombectomy treatments are often unable to remove sufficient clot to provide effective treatment in a single pass, and patients continue to suffer stroke symptoms even after the treatments, and multiple passes may be required. Treatments requiring multiple passes often increase procedure duration and/or blood vessel stress, elevating the risk of damage to the treated blood vessels. Presently available mechanical clot removal treatments also risk fragmenting the clot, creating emboli that can block smaller downstream vessels.

Of relevance to the disclosed technologies, one type of mechanical clot removal catheter uses rotating cutters and similar elements to break up clot and thrombus to enhance aspiration. While generally effective, it has been found that such rotating elements and their support structures can become entangled in fibrin when aspirating high fibrin clot, causing clogging of the clot-extraction lumen.

Blood clots that cause stroke are often heterogenous in composition with highly viscous portions that are resistant to aspiration using small diameter catheters of the type that are typically used in blood vessels supplying the brain. Attempts to remedy this challenge include the use of larger diameter (“large bore”) aspiration catheters that are matched in size to the inner diameter of the cerebral blood vessels being treated and/or the use of pulsatile aspiration pumps which is believed to increase the ingress of clot by increasing shear forces at the catheter tip. While sometimes effective, large-bore catheters cannot always access smaller target vessels and many clots resist aspiration even into large-bore catheters or are resistant to pulsatile aspiration.

A technique referred to as “corking” involves aspirating a short length of clot into an aspiration catheter tip. The result is that the clot is grasped but not fully aspirated by negative pressure at the tip of an aspiration catheter. The aspiration catheter is gently and slowly retracted to draw the clot into a larger guide or other catheter for extraction. While sometimes effective, corking risks clot break-up and downstream embolization.

For these reasons, it would be desirable to provide alternative and improved devices and methods for removing clot and thrombus from patients suffering from stroke and other conditions associated with vascular clotting. In particular, it would be desirable to provide mechanical clot removal catheters having an enhanced ability to remove clot rapidly, completely, and safely from the neurovasculature and other blood vessels and a reduced propensity to clog during use and, in particular in so doing, to lessen the tendency for rotating catheter elements to become entangled with fibrin during aspiration. It would be further desirable to accomplish this without significantly increasing the diameter of the clot removal catheters. At least some of these objectives would be met by the technologies described herein.

Background Art WO2023/219965; WO2023/219964; WO2025/038507; U.S. Pat. Nos. 4,646,736; 5,423,799; 6,454,779; 7,235,088; 7,981,128; 10,517,632; 10,835,272; 10,959,750; 10,960,178; 11,224,458; 11,931,055; 12,364,491; US2014/0324080; US2025/0228588; and CN110680463.

### BRIEF SUMMARY

The technologies disclosed herein provide methods, apparatuses, and systems for aspirating clot or thrombus (referred to herein collectively as clot) from a patient's blood vessels. Aspiration can be mechanically enhanced by applying a shear force to the clot while simultaneously drawing the clot into a clot-extraction lumen of an aspiration catheter. The technologies are particularly useful for removing clot from the neurovasculature (e.g., treatment of ischemic stroke) but will find use in the coronary (e.g., treatment of pulmonary embolism) and peripheral vasculatures (e.g., treatment of peripheral artery disease in the arms or legs) as well.

The shear force may be applied by a moving “shear” surface disposed at or near a distal opening of the catheter body through which the clot is being aspirated. As the clot is aspirated into and through the opening, the clot will engage the moving shear surface which applies a shear force to the clot which in turn will thin the clot and enhance aspiration, referred to herein as mechanical shear-thinning.

The moving surface may be provided in a number of ways as part of various structures and assemblies referred to generally herein as a “clot-disruption element,” “clot disruptor,” “occluder,” or simply “disruptor.” Examples of a clot-disruption element, disruptor and/or occluder includes, but are not limited to plates, discs, covers, shields, panels, blocks, and the like which are disposed at the distal opening and configured to expose clot to the moving surface. A clot disruption element may encompass any of foregoing terms and may refer to any element that rotates and revolves for removing clot from a vessel as described throughout the present disclosure. The moving surface may be planar and disposed transversely to the opening, e.g., perpendicularly to a longitudinal axis of the distal opening, so as to occlude a portion but not all of the distal opening, leaving an aspiration zone through which the clot can pass into a clot-extraction lumen.

The aspiration zone will typically form a flow constriction across the open distal region of the catheter, i.e., the aspiration zone will have a cross-sectional area which is less than that of open distal region in the absence of the clot-disruption element, disruptor or occluders. The clot is drawn therethrough the aspiration zone with a high velocity (but low volume) flow created by the constriction.

The moving surfaces of the clot-disruption elements, disruptors or occluders of the disclosed technologies may be disposed to rotate or revolve (i.e., orbitally rotate) within the open distal end of the aspiration catheter and apply shear to the clot that is being aspirated into the catheter tip and in particular applying shear to the meniscus of viscous clots. Applying shear to a localized region of clot within the meniscus reduces the viscosity due to shear thinning which occurs at the interface of the meniscus and at the rotating, revolving or other moving surface.

While in some instances the aspiration zone may be at a fixed location in the open distal end of the aspiration catheter, for example being located at a center of the open distal region of the catheter while the moving surface rotates or revolves thereabout, in most instances the aspiration zone will also be moving, and clot may be drawn through this moving aspiration zone, which may squish (e.g., laterally or radially compress) the clot as it enters the catheter. Dynamically changing the position of the aspiration zone relative to the inner wall of the catheter is believed to contribute to the shear thinning of the clot as it is aspirated into the clot-extraction lumen.

In specific examples, this dynamically changing aspiration zone comprises a crescent-shaped region or gap between a periphery of an occluder or disruptor that travels orbitally around the inner wall of the distal opening into the clot-extraction lumen. This process repeats itself continuously (e.g., constantly) as more and more clot is aspirated and shear-thinned against the rotating occluder or disruptor and then aspirated in the high velocity flow field.

The constricted aspiration zone is typically located in the distal opening of the aspiration catheter. In the disclosed embodiments, the constricted aspiration zone may be defined by or formed through a flow-blocking occluder or disruptor placed across the distal opening. In many embodiments, the occluder or disruptor will be a thin, flat plate that occludes flow through some or most of the distal opening but which presents minimum flow resistance through the aspiration zone, i.e., the aspiration zone acts as a flow orifice in restricting flow. For example, a disc placed across the distal opening may have a periphery sized or shaped to permit clot to bypass the disc and/or may have openings formed therethrough to allow clot to flow through the disc. The constricted aspiration zone thus created will typically be moved within, across, or over the area of distal tip, typically by revolving or rotating the disc or other flow-blocking plate, occluder or disruptor to revolve the aspiration zone in such a way to continuously displace and disrupt a stream of clot as it is aspirated through the moving aspiration zone into the clot-extraction lumen of the aspiration catheter.

Clot aspiration efficacy may be enhanced by reversing a direction in which the disc, occluder or disruptor, or other clot-disrupting element, for example a disc shaped structure, is rotated or revolved, typically by continuously alternating the rotational direction, often with a short pause between each reversal. Reversal frees any fibrin that may have attached itself to the clot-disrupting element or to the drive cable during rotation in a first direction. Once freed, this attached fibrin may then be aspirated.

Shear thinning of clot may also be enhanced by continuously accelerating and decelerating the rotation or revolution of the occluder or disruptor or other clot-disruption element, e.g., having a dynamically changing acceleration/deceleration during the majority of the time operating, rather than rotating at a constant speed. Such dynamic acceleration/deceleration is typically performed together with reversing the rotational direction, but in some cases could be used without changing the rotational direction. These technologies and techniques have been found to increase both a fluid velocity and dynamic pressure of the clot to both enhance clot uptake rate and decrease clot clogging in the tip.

The methods and apparatuses described herein may employ or include a clot-disrupting element that is configured to rotate around and/or across the distal region opening into the clot-extraction lumen (e.g., aspiration opening). For example, the occluder or disruptor or other clot-disrupting element may revolve or rotate in a plane that is transverse to the distal region, usually perpendicular to longitudinal axis of the clot-extraction lumen. The occluder or disruptor or other clot-disrupting element may at least partially occlude the opening into the clot-extraction lumen. As used herein, an occluder or disruptor or other clot-disrupting element may be configured as a plate, a disc, a cover, a shield, a panel, etc. The occluder or other clot-disruption element may have a distal face that is flat or curved. In some cases, the distal face is concave (e.g., rounded, bullet-shaped, etc.) from a top view in an axial direction. In some cases, the distal face of the occluder or disruptor or other clot-disruption element is concave. The distal face may be substantially smooth. Alternatively, the distal face may be textured, e.g., may include one or more channels and/or surface irregularities. The distal face of the occluder or disruptor or other clot-disruption element may be solid or, in some variations, may include one or more openings. In some cases, the occluder or disruptor or other clot-disruption element may have a ring shape. A “ring’ as used throughout the present disclosure may be interchangeably referred to as a “clip” or a “C-clip” as would be appreciated by one having ordinary skill in the art upon reading the present disclosure. The periphery of the occluder or disruptor or other clot-disruption element in the plane transverse to the aspiration opening may have a generally circular shape; alternatively, the occluder or disruptor or other clot-disruption element may have an oval or polygonal periphery (e.g., triangular, square, hexagon, etc.). For convenience, the occluder or disruptor or other clot-disruption element may be illustrated herein as a plate, for example, a disc, however it should be understood that any appropriate occluder or disruptor may be used, unless the context indicates otherwise.

In various embodiments, methods for removing clot from a patient's vasculature in accordance with the disclosed technologies comprise introducing an aspiration catheter into a blood vessel to position an open distal region of the aspiration catheter proximate clot. A flow-blocking structure is positioned transversely across the open distal region while leaving an open aspiration zone around or through the plate structure. The flow-blocking plate is revolved or rotated about a central axis of the open distal region, and a negative pressure is applied to a clot-extraction lumen to draw clot through the aspiration zone and into the clot-extraction lumen, thus moving the aspiration zone formed between the edge of the flow-blocking plate and the inner diameter of the aspiration opening. It has been found that aspirating clot through such a moving aspiration zone clot increases both a flow velocity and a dynamic pressure of the clot (relative to the flow velocity and the dynamic pressure when the plate structure is absent), which modifies the clot to both enhance a clot uptake rate and reduce clogging of the clot-extraction lumen.

In some instances, the flow velocity may be increased by a factor of at least 1.5, often 1.75 or more, 2 or more, 3 or more, 4 or more, or greater, and the dynamic pressure may be increased by a factor of at least 2, often 2.5 or more, 3 or more, 4 or more, 5 or more, or greater.

In some instances, revolving or rotating the plate structure causes the open aspiration zone to revolve about an axis of the open distal region.

In some instances, the open distal region may have a cross-sectional area in a range from about 1.2 mm² to 50 mm² (e.g., about 1 mm² to 6 mm², 1.2 mm² to 5.1 mm², 1.2 mm² to 10 mm², 5 mm² to 50 mm², 6 mm² to 50 mm², 10 mm² to 50 mm², etc.).

In some instances, the aspiration zone may have a cross-sectional area in a range from about 0.48 mm² to 40 mm².

In some instances, a cross-sectional area of the aspiration zone may have a cross-sectional area in a range from about 20% to 80% (e.g., about 25%-85%, about 20%-85%, about 30%-80%, about 35%-80%, about 40%-80%, about 45% to 80%, about 50%-80%, about 40%-75%, about 40%-70%, about 40%-65%, about 40%-60%, etc.) of a cross-sectional area of the distal region.

In some instances, the plate structure may comprise a disc.

In some instances, the disc may have a thickness in a range from 0.003 inches to 0.012 inches (i.e., 0.076 mm to 0.305 mm).

In some instances, the disc may be planar on at least a distal surface.

In some instances, the disc may have a rounded periphery, for example having a circular periphery.

In some instances, the disc may be convex in profile such that its center is closer to the catheter tip than its edge, and in some instances the center could extend beyond a plane of the distal opening while the edge remains at or recessed behind the plane of the distal opening. In this way, the center of the disc may exert a greater contact shear force against the clot meniscus than against the edges of the disc.

In some instances, the disc may have a width which is less than an inner diameter of the open distal region and the aspiration zone is located between an outer periphery of the disc and an inner wall of the open distal region. In such instances, the disc will typically be revolved about the axis of the open distal region to revolve the aspiration zone.

In some instances, the disc may have a width which is substantially equal to an inner diameter of the open distal region, and the aspiration zone may be located asymmetrically through the disc. In such instances, the disc will typically be rotated about the axis of the open distal region to revolve the aspiration zone.

By “revolve,” “revolved,” or “revolving,” it is meant that the aspiration zone, flow-blocking plate, disc, or other structure moves or is moved in a circular or orbital path about the central axis of the open distal region. In contrast, by “rotate,” “rotated,” or “rotating,” it is meant that plate structure, disc, or other structure spins or is spun about its own axis or center. Thus, when the flow-blocking plate is asymmetrically mounted within the open distal region, both the blocking plate and the aspiration zone created between a periphery of the blocking plate and the inner wall of the open distal region will revolve about center axis of the open distal region. In contrast, when the blocking plate is symmetrically located in the open distal region and has an asymmetrically located aspiration zone therethrough, the blocking plate can be rotated and only the aspiration zone will revolve to follow an orbital path.

In various embodiments, methods for removing clot from a patient's vasculature in accordance with the disclosed technologies comprise introducing an aspiration catheter having a clot-extraction lumen into a blood vessel to position an open distal region of the clot-extraction lumen proximate clot. A clot-disruption element, e.g. any of the plates, discs, covers, shields, panels, blocks or other occluders or disruptors described or in some instances open rings and other non-occluding clot-disruption elements, is rotated within the open distal region to cause an open gap region (e.g., an opening between a peripheral location on the clot-disruption element and the inner wall of the open distal region) to travel in an orbital path within the open distal region. A negative pressure is applied to a proximal end of the clot-extraction lumen to draw clot through the open gap region and into the clot-extraction lumen as the open distal gap region is travelling in the orbital path. The combination of aspiration with the moving open gap region may reshape clot thereby enhancing clot movement past the open gap region and into and through the clot-extraction lumen.

In some instances, revolving the occluder or disruptor or other clot-disruption element will increase both a flow velocity and a dynamic pressure applied to the clot relative to the flow velocity and the dynamic pressure when the is absent and the open distal region is unblocked. For example, the flow velocity may be increased by a factor of at least 1.5 and the dynamic pressure is increased by a factor of at least two.

In some instances, the open gap region may be diametrically opposed to a region on the periphery that lies closest to the inner wall of the open distal region.

In some instances, the occluder or disruptor or other clot-disruption element may have a circular periphery, e.g., may comprise a disc. The circular periphery may have an outer diameter in a range from 25% to 95% of an inner diameter of the rounded inner wall of the open distal region, typically being from 35% to 85%, often being from 50% to 75%.

In other instances, the occluder or disruptor or other clot-disruption element may have a non-circular periphery, for example being oval, polygonal, or irregular.

In some instances, the occluder or disruptor or other clot-disruption element may be revolved in a plane transverse to a longitudinal axis of the open distal region, where the plane may optionally be recessed behind the open distal region by a distance in a range from 0 to 10 mm, often from 0 to 5 mm, sometimes from 0 to 1 mm, and typically from 0.1 to 1 mm.

In instances where the clot-disrupting element comprises an occluder or disruptor, the occluder or disruptor may include a distal face that orbits the distal opening into the aspiration lumen in a plane that is parallel with a plane of the opening into the aspiration lumen and is within +/−10 mm (e.g., within +/−9 mm, within +/−8 mm, within +/−7 mm, within +/−6 mm, within +/−5 mm, within +/−4 mm, within +/−3 mm, within +/−2 mm, within +/−1 mm, within +/−0.5 mm, etc.) of the plane of the opening. In some cases the distal face of the occluder or disruptor may orbit the opening into the aspiration lumen in the same plane as the opening into the aspiration lumen, optionally being inhibited from extending distally beyond the plane of the opening into the aspiration lumen, e.g., by a retention element, such as a retention protrusion, retention ring, or the like located distal to the distal face of occluder or disruptor.

In some instances, the occluder or disruptor or other clot-disruption element may comprise a substantially solid disc disposed in the open distal region and oriented in a plane normal to the central axis.

In some instances, the substantially solid disc may be free from gaps across its surface.

In some instances, the substantially solid disc may have a generally circular periphery. In other instances, the substantially solid disc has a non-circular geometry.

In general, the occluder or disruptor or other clot-disruption element (e.g., disc, ring, etc.) may be part of an occluder or disruptor or other clot-disruption assembly that is coupled to a drive element, such as a drive cable, gears, or the like, that is configured to rotate and/or revolve the occluder or disruptor or other clot-disruption element. Such assemblies may include a retention element, such as retention protrusion (e.g., ring, split ring, wing(s), arm(s), etc.) and a connector (e.g., strut, arm, etc.) that may connect the occluder or disruptor or other clot-disruption element to the drive element, such as a drive cable.

In some instances, revolving the occluder or disruptor or other clot-disruption element may comprise rotating the drive cable or other element which has a distal end coupled to an outer peripheral location on a proximal side of the occluder or disruptor or other clot-disputing element. For example, the proximal side of the occluder or disruptor or other clot-disruption element may be attached to a retention member, such as a retention ring or other element that is rotatably mounted in an annular guide channel of an annular guide assembly disposed on an inner wall of the open distal region. The annular guide channel maintains the longitudinal position of the retention member and the entire clot-disruption assembly relative to the open distal region as the occluder or disruptor or other clot-disruption element is being revolved.

Exemplary annular guide assemblies may include a guide element that movably engages with the retention element(s) to allow the occluder or disruptor assembly, and therefore the occluder or disruptor, to revolve and/or rotate relative to the opening into the aspiration lumen. In some cases, the annular guide assembly may include one or more guide surfaces that may be configured as channels, ramps, protrusions, rings, split-rings, etc. that are positioned radially within the aspiration lumen so that it may engage with, and in some cases removably engage with, the occluder or disruptor assembly to maintain the position of the occluder or disruptor relative to the opening into the lumen, while allowing the occluder or disruptor assembly to rotate. In some cases, the annular guide assembly may act as a stop to prevent further distal advancement of the occluder/occluder assembly/disruptor. For example, the stop may be a distal lip or edge within the lumen that may extend at least partially around the inner diameter of the lumen.

In some instances, the drive cable may be directly attached to the retention element.

In other instances, the drive cable may be connected to the retention element through an axial connector, such as a strut, post, arm, or the like, to which the retention element and/or the occluder or disruptor or other clot-disruption element is attached.

In some instances, the retention ring or other element may be positioned in the annular guide channel of the annular guide assembly prior to introducing the aspiration catheter to the blood vessel.

In some instances, the occluder or disruptor or other clot-disruption assembly may be pre-positioned in the aspiration catheter prior to introducing the catheter into to the blood vessel.

In some embodiments, the occluder or disruptor or other clot-disruption assembly may be positioned in the annular guide prior to the aspiration catheter being advanced into the patient's vasculature.

In other embodiments, the occluder or disruptor or other clot-disruption assembly may be positioned proximal of the annular guide assembly within the clot extraction lumen prior to the aspiration catheter being advanced into the patient's vasculature. The occluder or disruptor or other clot-disruption assembly is fully advanced into the annular guide assembly only after the distal end of catheter is approaching the clot at which point the occluder or disruptor or other clot-disruption assembly may be moved into the annular guide assembly and the tip of the aspiration catheter advanced into the clot. This technique may minimize perturbation of the clot prior to aspiration.

In other embodiments, the occluder or disruptor or other clot-disruption assembly may be locked into position using the retention ring when the aspiration catheter tip is proximal to the clot and then the assembly may be advanced to and slightly into the clot as a unit. This technique can be preferred as it may cause the least perturbation of the clot prior to aspiration.

In some instances, the retention ring or other element may be configured to elastically deform to releasably engage or lock into the annular guide assembly. For example, the retention ring or other element may elastically contract as it is advanced into the guide channel.

In some instances, the retention ring or other element may have a structure that inhibits accidental dislodgement from the annular guide assembly. For example, the annular guide channel may have a proximal annular side or annular ramp that is steeper than a distal annular side or ramp.

In some instances, revolving the occluder or disruptor assembly or other clot-disruption structure may be configured to reverse its rotational direction of revolution periodically to enhance aspiration, for example the direction of revolution may be reversed after a time period typically in a range from about 0.1 sec to 10 sec.

In various embodiments, systems for removing clot from a patient's vasculature in accordance with the disclosed technologies comprise a catheter body having a clot-extraction lumen extending from a proximal end to an open distal region, where the proximal end is configured to be attached to a vacuum source and the open distal region has a central axis and typically has a rounded inner wall contiguous with the clot-extraction lumen. The occluder or disruptor or other clot-disruption element comprises a plate structure disposed transversely across at least a portion of the open distal region and which may be configured to define a flow-blocked zone and an open aspiration zone though the open distal region. A driver, typically comprising or consisting of a drive cable or shaft, is configured to revolve or rotate the plate structure about the central axis to cause the open aspiration zone to travel in an orbital path about the central axis of the open distal region. Applying a negative pressure to the proximal end of the clot-extraction lumen draws a meniscus of the clot into the open aspiration zone and subjects the clot to shear thinning as the thinned clot is aspirated into the clot-extraction lumen at a high velocity.

In some instances, the plate structure may be configured to generate a clot flow through the open aspiration zone having an increased flow velocity and an increased dynamic pressure relative to a flow velocity and a dynamic pressure of a clot flow into the open distal region in the absence of the plate structure.

In some instances, the plate structure may be asymmetrically positioned in the open distal region and is revolved about the central axis and wherein the aspiration zone is located between a peripheral edge of the plate structure and the rounded inner wall and follows an orbital path as the plate structure is revolved. For example, the driver may comprise any conventional drive element or structure, typically a drive cable or shaft, having a distal end coupled to the plate structure at or near a peripheral edge thereof at a location diametrically opposed to the open aspiration zone, where rotation of the drive shaft causes a distal length of the drive shaft to precess about the central axis.

In other instances, the plate structure may be symmetrically positioned in the open distal region and may be rotated about the central axis, and the aspiration zone may comprise an off-axis, flow-restricting aperture through the plate structure.

In some instances, the plate structure may comprise a planar structure oriented in a plane normal to the central axis.

In some instances, the open distal region may have a cross-sectional area in a range from about 1.2 mm² to 50 mm² and the aspiration zone may have a cross-sectional area in a range from about 0.48 mm² to 40 mm².

In some instances, the aspiration zone has a cross-sectional area in a range from about 40% to 80% of a cross-sectional area of the distal region.

In some instances, the plate structure may comprise a disc, wherein the disc may have an average thickness in a range from 0.1 mm to 1 mm, and may be planar on at least a distal surface.

In some instances, the disc may have a rounded periphery, typically a circular periphery, and may have a width which is less than an inner diameter of the open distal region and the aspiration zone is located between an outer periphery of the disc and an inner wall of the open distal region, typically having an average width or diameter in a range from 25% to 95% of the inner diameter of the open distal region of the clot-extraction lumen.

In some instances, the driver may be configured to revolve the disc to in turn revolve the aspiration zone.

In some instances, the disc may have a width which is substantially equal to an inner diameter of the open distal region, where the aspiration zone Is typically located asymmetrically through the disc. In such instances, the driver is typically configured to rotate the disc about the central axis of the open distal region which causes the aspiration zone to revolve about the axis.

In some instances, the disc may be substantially solid and free from gaps across its surface.

In some instances, the substantially solid disc has a rounded peripheral edge.

In some instances, a distal face of the disc or other plate may be contoured, for example having a convex distal face with a center that protrudes distally and may enhance contact with and shearing of the clot meniscus.

In some instances, the systems may further comprise a clot disruptor element such as a disc or plate retention structure or assembly, typically a circular retention ring coupled to and disposed proximally of the disc or plate structure, wherein the retention ring is received in an annular guide channel disposed on an inner wall of the open distal region.

In such instances, the circular retention ring may have an outer diameter approximately equal to an inner diameter of the open distal region of the clot-extraction lumen and comprises a distal bearing surface which engages a distal bearing surface on the guide channel, and the circular retention ring may further comprise a distal bearing surface which engages a proximal bearing surface on the guide channel. The distal and proximal bearing surfaces on the annular guide channel are typically axially spaced apart to form an annular slot for rotationally receiving the retention ring.

In some instances, the plate structure and the retention ring may be joined by an axial connector, such as a strut, post, arm, or the like. A distal end of the axial strut or other connector may be connected to a connection point on the periphery of the retention ring and have an offset portion, e.g., a dog leg, configured to position the connection point immediately adjacent to the inner wall of the open distal region as the plate structure is revolved or rotated. The dog leg may help to position the retention ring connection point immediately adjacent the inner wall of the catheter, which may increase the squishing leverage (e.g., the lateral/radial compression) of the occluder or disruptor against the inner wall of the catheter, and consequently on the clot.

In some instances, the retention ring may be split or comprise a gap configured to allow the retention ring to elastically contract as the retention ring is inserted into the annular guide channel. In such cases, the retention ring may have two wings or arms extending transversely from the axial connector.

In general, the apparatuses described herein may be configured to be actuated by driving rotation of a drive element, which may also be referred to herein as a drive transmission or simply transmission, and may be a drive cable, shaft, or the like. The drive element may be driven by a proximal driver that may include a motor, that may rotate the drive element. The motor may be electrically-driven or driven by other means such as pressurized air. In some cases the driver may be integrated with the occluder or disruptor or other clot-disruption assembly. In some cases, the driver may be a hand-held rotatory driver. Hand-held rotary drivers and motor drivers may be separate from the occluder or disruptor or other clot-disruption assembly and may be coupled to the proximal end region of the drive element to control rotation of the occluder or disruptor.

In some instances, the driver may comprise a drive cable or other drive element a motor configured to rotate the drive cable about an axis parallel to the central axis. The driver may further comprise an outer sleeve having a longitudinal passage therethrough, wherein the outer sleeve is non-rotatably disposed over the drive cable, and at least a portion of the drive cable may be covered with a lubricous cover or coating. In some instances, the outer sleeve may also have a lubricious coating. In some instances the outer sleeve may provide additional column strength to the otherwise flexible drive cable or other drive element.

In some instances, the driver may comprise a hand-held rotary driver, typically including (1) a handle detachably or permanently fixed to the drive cable and/or the outer sleeve and (2) a power module including the motor detachably secured to the handle, whereby the handle may be used to manipulate the drive shaft and sleeve in the clot-extraction lumen of the aspiration catheter prior to attaching the power module.

In some instances, the handle may comprise an input drive connector connected to the drive cable and may further comprise a flush port fluidly connected to the outer sleeve, typically to deliver a fluid to annular space between the drive cable and an inner wall of the outer sleeve.

In some instances, the power module may include a battery, control circuitry, and an output drive connector connected to the motor.

The driver and clot-disruption assemblies may include a connector therebetween. For example, the clot-disruption assembly may include a proximal drive connector (input connector) that may be configured to engage a distal drive connector (output connector) of the driver.

In some instances, the input and output drive connectors may comprise magnetic coupling elements, typically including a “key” or other alignment feature(s) to assure that there is no slippage in the rotary transmission, i.e., rotation of the driver and the clot-disruption assemblies remain synchronous.

In various embodiments, the technology disclosed herein provides a system for removing clot from a blood vessel. The system comprises a catheter body including a proximal end and a clot-extraction lumen. The clot-extraction lumen has an open distal region and a central axis therethrough. A clot-disruption structure, typically having a distal occluder or disruptor or other clot-disruption element, may be disposed proximate (usually being flush or retracted a small distance) the open distal region of the clot-extraction lumen, and a driver is configured to revolve the clot-disruption structure about the central axis of the open distal region of clot-extraction lumen as the clot is aspirated into the open distal. The clot-disruption structure is configured to modify the structure of the clot as the clot is aspirated into the open distal region while the clot-disruption structure is revolved about the central axis of the open distal region.

By “revolve” and “revolving” it is meant that a center point of the occluder or disruptor or other clot-disruption element moves in a circular pattern about the central axis of the open distal region of the clot-extraction lumen so that the occluder or disruptor or other clot-disruption element sweeps an annular path, as illustrated for example in FIGS. 4A to 4C hereinbelow. Since the center point of the occluder or disruptor or other clot-disruption element may be radially offset from the central axis, an asymmetric annular gap is created between an outer periphery of the occluder or disruptor or other clot-disruption element and an inner wall of the open distal region of the clot-extraction lumen.

The asymmetric annular opening thus moves in an annular, circular path about the open distal region and applies shear forces to the clot meniscus to cause shear thinning to promote aspiration as a negative pressure is applied to the clot extracting lumen. In some instances, the clot may be disrupted or rendered into smaller segments, as described in more detail below. In other instances, fibrin may be displaced, separated, or otherwise modified to facilitate aspiration of the clot. Such possible mechanisms of action are not meant to be limiting in any way and are offered only to help understand the present technology.

In the disclosed examples of the present technologies, the occluder or disruptor or other clot-disruption element will often rotate about its own center point in addition to the center point travelling in a circular pattern about the central axis of the open distal region of the clot-extraction lumen. Such additional rotation of the occluder or disruptor or other clot-disruption element may further facilitate conditioning and aspiration of the clot but is not considered essential for performance of the disclosed technologies.

In some instances, the clot-disruption element may be other than an occluder or disruptor, for example comprising a looped or other element having an open central region, sometimes referred to as a clot-disruption ring or “halo.” Such open structures provide at least two paths for the clot to be drawn into the clot-extraction lumen: a first path entering through the opening and a second bypassing an outer periphery of the clot-disruption ring, i.e., between an outer peripheral edge of the clot-disruption ring and an inner wall of the open distal region of the clot-extraction lumen.

In other instances, the occluder or disruptor or other clot-disruption element will comprise a substantially solid or disc-like element which provides only a “bypass” path between an outer peripheral edge of the disc-like element and an inner wall of the open distal region of the clot-extraction lumen. Surprisingly, it has been found that in many instances, the disc-like clot-disruption or other substantially solid element has a performance equal or superior to that of a ring-like occluder or disruptor or other clot-disruption element. By “substantially” solid, it is meant that the occluder or disruptor or other clot-disruption element will be solid and impenetrable by blood or clot over at least 90% of its surface area, usually over at least 95% of its surface area, and most often solid and free from holes, gaps, or other passages over the entire are inward from its periphery. The solid disc-like clot-disruption elements will typically have rounded edges, although in other embodiments might have squared or sharpened edges.

In some instances, such solid disc-like occluders or disruptors and other clot-disruption elements may have non-planar distal faces with raised, wave-like edges that may be configured to speed the movement of clot segments into the catheter as the raised edges catch and propel clot during rotation of the solid disc.

The aspiration catheters of the disclosed technologies can improve efficacy by reducing clogging resulting from fibrin becoming entwined in the clot-extraction lumen, for example by minimizing structure present in the lumen and particularly by minimizing structure present in the center of the open distal region of the clot-extraction lumen. In this way, the catheter lumen patency can be improved.

In specific examples, as described in more detail hereinafter, the occluder or disruptor or other clot-disruption element will be located on a distal end of the clot-disruption structure and be asymmetrically disposed within the open distal region of the clot-extraction lumen while a proximal portion of the clot-disruption structure (such as a retention ring described hereinafter) will be symmetrically disposed in the open distal region of the clot-extraction lumen. In this way, the distal occluder or disruptor or other clot-disruption element will follow a radially offset annular path and the proximal portion can rotate while being constrained to the inner wall of the open distal region of the clot-extraction lumen.

A peripheral location on the proximal portion of the clot-disruption structure can be connected to a distal end of a rotating drive cable. Rotation of the drive cable will cause the distal end of the drive cable to “revolve” along a circular path adjacent to an inner wall of the open distal region of the clot-extraction lumen so that blockage of the distal end of the aspiration path by the drive cable is minimized. Such drive cable rotation, in turn, causes the occluder or disruptor or other clot-disruption element to revolve as it follows an annular path in the open distal region of the clot-extraction lumen. In some instances, clot aspiration may be improved by rapidly alternating the direction of drive cable rotation between clockwise and counterclockwise with short pauses between directional changes. For example, the direction of revolution may be reversed every 0.1 sec to 10 sec, sometimes from 0.5 sec to 10 sec, sometimes from 1 sec to 5 sec. In this way, the drive cable and clot-disruption structure is nearly continuously exposed to acceleration and deceleration rather than continuous velocity. The short pauses between directional changes may allow strands of fibrin to travel into the catheter past the clot-disruption structure. It will be appreciated that due to frictional forces between the rotating drive cable and clot-disruption structure assembly and the inner wall of the catheter, the rotational acceleration and deceleration of the clot-disruption structure may be less than that of the proximal end of the drive cable. Therefore, to achieve adequate acceleration and deceleration of the occluder or disruptor or other clot-disruption element, the proximal end of the drive cable may need to be rotated to a greater extent.

In alternative embodiments, the clot-disruption structure will be revolved in only one direction, either with or without pauses, acceleration and deceleration of the rotational speed, or other changes in the rotational speed.

The occluder or disruptor or other clot-disruption element of the clot-disruption structure will have a width, usually a diameter for circular occluder or disruptor or other clot-disruption elements, less than the inner diameter of the open distal region and will typically comprise a planar structure oriented in a plane normal to the central axis of the clot-extraction lumen. As the center of the occluder or disruptor or other clot-disruption element will be radially offset from the central axis of the open distal region, a generally crescent-shaped gap or opening will be left on a side of the occluder or disruptor or other clot-disruption element which is diametrically opposed to side where the clot-disruption structure is connected to the driver.

In other instances, a distal face of the occluder or disruptor or other clot-disruption element could have a curve, wave, or other small deviation from being true planar.

A distal surface of the occluder or disruptor or other clot-disruption element will be located close to or at the open distal region of the clot-extraction lumen, typically being in a range from 0 to 10 mm, 0 to 8 mm. 0 to 6 mm, 0 to 4 mm, and 0 to 2 mm; sometimes being in a range from 0.5 mm to 10 mm, 0.5 mm to 8 mm. 0.5 mm to 6 mm, 0.5 mm to 4 mm, and 0.5 mm to 2 mm, and often being in a range from 1 mm to 10 mm, 1 mm to 8 mm. 1 mm to 6 mm, 1 mm to 4 mm, and 1 mm to 2 mm. In some embodiments the distal surface of the shearing element will be flush with the open distal region of the clot-extraction lumen.

While the clot-disruption structure may rotate at a constant and/or smoothly or continuously accelerating rate, it has been found that in some instances rapid rotational acceleration, deceleration, and alternating the rotational direction of the occluder or disruptor or other clot-disruption element may provide a more efficient break-up of the clot and thrombus than rotation in one direction only, resulting in efficient clot/thrombus aspiration with reduced clogging due to fibrin and other causes.

In some instances, the occluder or disruptor or other clot-disruption element comprises circular or similar open “looped” structures that provide separate paths for the clot to enter the open end as the clot/thrombus is being aspirated into the clot-extraction lumen. A first path is through the opening region in the occluder or disruptor or other clot-disruption element while a second path is located between an outer periphery of the occluder or disruptor or other clot-disruption element and an inner luminal wall of the open distal region. As the occluder or disruptor or other clot-disruption element is revolved, both paths will be continuously displaced providing a more controlled rending or break-up of the clot than would be provided by an occluder or disruptor or other clot-disruption element that rotates only.

In some instances, the occluder or disruptor or other clot-disruption element may comprise an at least partially looped element disposed in the open distal region and oriented in a plane normal to the central axis.

In some instances, the at least partially looped element may circumscribe an inner clot passage area and may define an asymmetric outer annular clot passage area disposed between an outer periphery of the loop and an inner wall of the open distal region.

In some instances, the at least partially looped element may comprise a continuous ring structure. The continuous ring structure will usually have a circular geometry but in other cases could have a non-circular geometry, such as oval, polygonal, or irregular.

In some instances, the looped element may have an average width thickness in a range from 0.1 mm to 1 mm, often in a range from 0.1 mm to 0.5 mm.

Circular, ring-like occluder or disruptor s or other clot-disruption elements will typically have an outer diameter in a range from 25% to 95% of an inner diameter of the open distal region of the clot-extraction lumen, usually having an outer diameter in a range from 35% to 85% of an inner diameter of the open distal region of the clot-extraction lumen, sometimes having an outer diameter in a range from 50% to 75% of an inner diameter of the open distal region of the clot-extraction lumen.

In other instances, the occluder or disruptor or other clot-disruption element comprises a substantially solid disc disposed in the open distal region and oriented in a plane normal to the central axis. The substantially solid disc will typically be free from gaps across its surface, but in some cases may have small discontinuities as discussed above.

In some instances, substantially solid disc may have a generally circular geometry.

In other instances, the substantially solid disc may a non-circular geometry.

In other instances, the substantially solid disc may be convex in profile.

In some instances, the substantially solid disc has an average width thickness in a range from 0.1 mm to 1 mm.

In some instances, the substantially solid disc has an average width thickness in a range from 0.1 mm to 0.5 mm.

In some instances, the substantially solid disc has an outer diameter in a range from 25% to 95% of an inner diameter of the open distal region of the clot-extraction lumen, usually having an outer diameter in a range from 35% to 85% of an inner diameter of the open distal region of the clot-extraction lumen, sometimes having an outer diameter in a range from 50% to 75% of an inner diameter of the open distal region of the clot-extraction lumen.

In some instances, the proximal portion of the clot-disruption structure may comprise a retention element disposed proximally of the occluder or disruptor or other clot-disruption element, where the retention element may comprise a circular or other retention ring having an outer diameter approximately equal to an inner diameter of the open distal region of the clot-extraction lumen. The retention ring will also have a distal bearing surface which engages a proximal bearing surface on a distal stop band disposed on an inner wall of the clot-extraction lumen in the distal open end. The distal stop band limits distal travel of the clot-disruption structure, typically preventing protrusion of the occluder or disruptor or other clot-disruption element beyond the distal end of the catheter.

In many embodiments, a proximal stop band will also be provided in the distal open end on the inner wall of the clot-extraction lumen. The proximal stop band will have a distal bearing surface configured to engage a proximal bearing surface on the retention ring. The distal and proximal stop bands will be axially spaced apart to form an annular slot for rotationally receiving the retention ring of the clot-disruption structure. In this way, axial movement of the clot-disruption structure is axially constrained without impeding the ability of the clot-disruption structure to rotate as it is driven by the driver.

The inner diameters of the stop bands may have slightly different inner diameters. For example, the inner diameter of the proximal stop band may have larger inner diameter to allow elastic retention rings to reversibly (elastically) pass through the proximal stop band while a smaller distal opening stops or inhibits further passage through distal stop band. Specific inner diameters and will depend on the overall all catheter dimensions. Exemplary distal: proximal diameters for 0.071 inch (i.e., 1.80 mm) inner diameter cerebral aspiration catheters include 0.060 inches to 0.062 inches (i.e., 1.52 mm to 1.57 mm) and 0.064 in to 0.065 in (i.e., 1.6256 mm to 1.651 mm).

While the retention rings will often have a continuous periphery (comprise a complete circle) and be substantially non-collapsible (withstand radially inward forces without significant loss of diameter), in many cases it will be desirable to configure the retention ring to elastically contract when exposed to radially inward forces, e.g. when being advanced past the proximal stop bend when that band is present. For example, the retention ring (and optionally other parts of the clot-disruption structure) may be formed from an elastic material, such a spring stainless steel or a nickel-titanium alloy, with a peripheral gap to allow elastic compression and expansion.

In some instances, the occluder or disruptor or other clot-disruption element and the retention ring or other element are joined by an axial strut, where for example the retention ring may be disposed proximally of the occluder or disruptor or other clot-disruption element by a distance in a range 0 mm to 15 mm, usually 0.5 mm to 15 mm, typically from 1 mm to 10 mm.

Exemplary distances may be in ranges from 0.5 mm to 1.5 mm and from 1.5 mm to 2.5 mm. In other cases, the occluder or disruptor or other clot-disruption element and the retention ring or other element may be joined directly together with no axial offset.

A distal end of the axial strut may be connected to a connection point on the periphery of the retention ring or other element. The axial strut will usually be configured to pass over the distal stop band, for example having a dog leg configured to position the connection point immediately adjacent to an inner surface of the open distal region of the catheter body as the clot-disrupting structure is revolved.

In some instances, the driver may comprise a drive cable and a motor configured to rotate the drive cable about an axis parallel to the central axis, where for example a distal end of the drive cable may have a fixed point of attachment on a peripheral edge of the occluder or disruptor or other clot-disruption element and wherein the fixed point of attachment may be radially offset from the central axis of the open distal region of clot-extraction lumen so that the occluder, disruptor, or other occluder or other clot-disruption element simultaneously revolves about the central axis and rotates about a center point of the occluder or disruptor or other clot-disruption element.

In some instances, the driver may further comprise an outer sleeve having a longitudinal passage therethrough, wherein the outer sleeve is non-rotatably disposed in the clot-extraction lumen and the drive cable is rotatably mounted in the longitudinal passage, where the drive cable is typically rotatably mounted in the clot-extraction lumen.

While the outer sleeve will be configured not to rotate along at least most of its proximal length, and often over its entire length, in some instances a distal portion of the outer sleeve may be configured to rotate with the drive cable so that the outer sleeve may be positioned as closely as possible to the retention ring as possible without engaging against the ring. For example, the sleeve may be “discontinuous” with the distal-most portion, e.g., several centimeter, being fixedly attached to the drive cable to prevent or mitigate sliding and rotate with the drive cable. The proximal portion would remain non-rotatably disposed about the drive cable.

At least a proximal portion of the outer sleeve will define the outer sleeve an annular space between drive cable and an inner wall of the outer sleeve to allow flushing.

In some instances, at least a portion of the drive cable may be covered with a lubricous cover or coating.

In some instances, at least a portion of the occluder or disruptor or other clot-disruption element may be covered with a lubricious cover or coating.

In some instances, at least a portion of the outer sleeve may be covered with a lubricious coating.

In some instances, the driver may be further configured to apply a distal biasing force on the drive cable to maintain forward positioning of the occluder or disruptor or other clot-disruption element.

In alternative embodiments, the retention ring or other element may be axially constrained to maintain positioning of the clot-disrupting structure in the absence of a biasing force provided by the drive cable. As described previously, the retention ring may be held in an annular guide channel usually disposed between axially spaced-apart stop bands. In such instances, the retention ring will preferably be configured to elastically contract so that it can be passed over the proximal stop band as the clot-disruption structure is axially advanced in the clot-extraction lumen.

In further embodiments, the systems of the disclosed technologies may comprise a detachable handle including the motor, where the handle is configured to rotationally drive the driver and non-rotationally secure the catheter body. In preferred instances, the handle may comprise any or all of a flush port, a battery, and control circuitry. In preferred instances, the handle may comprise a distal housing detachably connectable to a proximal housing, where the distal housing comprises a flush port and a proximal drive connector and the proximal housing comprises the motor, control circuitry, batteries, and a distal drive connector. Typically, the proximal and distal drive connectors comprise magnetic coupling elements.

In various embodiments, the technology disclosed herein provides a method for removing clot from a blood vessel. The method comprises introducing an aspiration catheter into a blood vessel to position an open distal region of the aspiration catheter proximate clot. A negative pressure is applied to a clot-extraction lumen to draw clot into the clot-extraction lumen through the open distal region, and an occluder or disruptor or other clot-disruption element is revolved about a central axis of the open distal region to condition the clot to facilitate passage of the clot into the open distal region of the clot-extraction lumen. In some cases, particularly when using a ring-like occluder or disruptor or other clot-disruption element, the clot/thrombus may be “rended” or otherwise broken into pieces as it is drawn into the clot-extraction lumen. In other cases, particularly when using a solid, disc-like occluder or disruptor or other clot-disruption element, intake of the clot/thrombus is facilitated with reduced break-up of the clot/thrombus. A solid disc-like clot disruption element may result in a faster update of the clot into the aspiration lumen.

In some instances, the method may further comprise rotating the occluder or disruptor or other clot-disruption element as it is being revolved.

In some instances, the method may comprise rapidly revolving the clot-disruption structure in alternating clockwise and counterclockwise directions such that the drive cable and clot-disruption structure are subject to nearly continuous acceleration and deceleration rather than continuous velocity. In such instances, the duration of rotation in one direction may be different from the duration in the opposite direction and/or the rate of rotational acceleration and/or declaration may differ in each direction. For example, the direction of revolution may be reversed every 0.1 sec to 10 sec, sometimes from 0.5 sec to 10 sec, sometimes from 1 sec to 5 sec, often being controlled based on average or maximum rotational speed, acceleration rate, deceleration rate, reverse direction, or other factors.

A brief (e.g., 0.5-2.0 second) pause between successive rotations may be desirable to allow strands of fibrin to flow unobstructedly past the clot-disruption structure and into and through the catheter extraction lumen. If fibrin-containing clot has wound around the clot-disruption structure, reversing the direction may lead to desirable unwinding. Once unwound, the brief pause would enable flow of the unwound or loosely wound fibrin-containing clot prior to rotation in the opposite direction.

In other instances, the methods may comprise rapidly revolving the occluder or disruptor or other clot-disruption element in a single direction (clockwise or counterclockwise), at either a constant or a varying rotational rate. When varying the rotational velocity, a brief pause in rotation between speed-up and slowdown may be desirable.

In most instances, the occluder or disruptor or other clot-disruption element will have a circular periphery with a diameter in in a range from 25% to 95% of an inner diameter of the open distal region of the clot-extraction lumen, usually having an outer diameter in a range from 35% to 85% of an inner diameter of the open distal region of the clot-extraction lumen, sometimes having an outer diameter in a range from 50% to 75% of an inner diameter of the open distal region of the clot-extraction lumen.

In some instances, the occluder or disruptor or other clot-disruption element will have a semi-circular or other rounded peripheral edge. In other instances, however, the peripheral edge may be squared, tapered, sharpened, serrated, sharpened, irregular or have other cross-sectional shapes intended for particular purposes.

In some instances, the occluder or disruptor or other clot-disruption element will revolve in an annular path in a plane normal to the central axis of the open distal, wherein the plane may for example be disposed proximally of a plane of the open distal region by a distance in a range 0 mm to 30 mm, often from 0.5 mm to 30 mm, more often from 0.5 mm to 15 mm, typically from 1 mm to 10 mm. Exemplary proximal offsets may be in ranges from 0.5 mm to 3 mm, usually from 1.5 mm to 2.5 mm. In other instances, the occluder or disruptor or other clot-disruption element may be aligned with the open distal region of the clot-extraction lumen. In other embodiments, the occluder or disruptor or other clot-disruption element may extend distally beyond the open distal region of the clot-extraction lumen.

In some instances, the occluder or disruptor or other clot-disruption element may comprise a ring, i.e. the occluder or disruptor or other clot-disruption element may have a circular periphery which surrounds an open central portion so that clot passes both through the open central portion and between the outer circular periphery and an inner wall of the open distal region.

In other instances, the occluder or disruptor or other clot-disruption element may comprise a non-circular periphery, which may be partially or fully across its center.

In some instances, the occluder or disruptor or other clot-disruption element may comprise an at least partially looped element disposed in the open distal region and oriented in a plane normal to the central axis, where, for example, the occluder or disruptor or other clot-disruption element comprises a ring.

In other instances of the methods described herein, the occluder or disruptor or other clot-disruption element comprises a substantially solid disc disposed in the open distal region and oriented in a plane normal to the central axis. The substantially solid disc will typically be free from gaps across its surface, but in some cases may have small discontinuities as discussed above.

In some instances, revolving the occluder or disruptor or other clot-disruption element structure may comprise rotating a drive cable having a distal end attached to a proximal surface of the occluder or disruptor or other clot-disruption element, where for example revolving the occluder or disruptor or other clot-disruption element may comprise applying a distal biasing force to engage a distal bearing surface of the occluder or disruptor or other clot-disruption element against a proximal bearing surface of a distal stop band disposed on an inner luminal wall of the open distal region while the occluder or disruptor or other clot-disruption element is being revolved.

In other instances, revolving the occluder or disruptor or other clot-disrupting structure may comprise constraining a retention ring or other element configured to be received and rotate in an annular guide channel disposed on an inner wall of the open distal region while the clot-disrupting structure is being revolved. For example, the occluder or disruptor or other clot-disrupting structure may be advanced in a distal direction through the clot-extraction lumen to position retention ring of the clot-disrupting structure in the annular guide channel prior to applying the negative pressure. Often, the retention ring of the clot-disrupting structure elastically contracts as it is advanced past a proximal stop band in the open distal region of the clot-extraction lumen.

Thus, described herein are apparatuses and methods for removing clot material in which an occluder or disruptor (e.g., disc, plate, etc.) orbitally moves across the aspiration opening into the aspiration lumen of the catheter. For example, a method of removing a clot material may include: positioning an aspiration catheter so that an opening into an aspiration lumen of the aspiration catheter is proximate to a clot material; moving an occluder or disruptor in a plane that is parallel to the opening of the aspiration catheter in an orbital manner so that an opening into the aspiration lumen between the occluder or disruptor and an inner wall of the aspiration opening moves around the aspiration opening; and applying suction while moving the occluder or disruptor to draw the clot material through the moving opening into the aspiration lumen between the occluder or disruptor and an inner wall of the aspiration opening.

The opening into the aspiration lumen may be at the distal end of the aspiration catheter. Positioning the aspiration catheter proximate to the clot (e.g., clot material) may generally include positioning the opening near, adjacent to, or in contact with the clot material. The aspiration catheter may be pre-loaded within the aspiration lumen to the plane that is parallel to the opening into the aspiration lumen, or it may be moved into position (e.g., through the lumen), during or after positioning the opening into the aspiration catheter proximate the clot material. For example, any of these methods may include positioning the occluder or disruptor in the plane by inserting the occluder or disruptor through the lumen of the aspiration catheter prior to moving the occluder or disruptor in the plane that is parallel to the aspiration opening.

The plane that is parallel to the opening of the aspiration catheter may be at or slightly proximal to the opening (e.g., between about 0 mm and 2 cm, between about 0-15 mm, between about 0-10 mm, between about 0-9 mm, between about 0-8 mm, between about 0-7 mm, between about 0-6 mm, between about 0-5 mm, between about 0-4 mm, between about 0-3 mm, between about 0-2 mm, between about 0-1 mm, etc.), but may configured so that at least the side(s) of the occluder or disruptor is/are prevented from extending out of the opening. Any of these methods may include maintaining the occluder or disruptor at or just proximal to the opening of the aspiration catheter, e.g., as the clot material is aspirated. This may include maintaining the occluder or disruptor in position while further advancing and/or withdrawing the catheter. Alternatively, the occluder or disruptor may be withdrawn proximally, as described in greater detail herein, before or during repositioning of the catheter for removal of additional clot from the body and/or for removal of the catheter from the body.

The occluder or disruptor may be moved in the plane by rotating and/or by revolving. In some cases moving the occluder or disruptor comprises moving a disc-shaped occluder or disruptor.

In general, moving the occluder or disruptor in the plane that is at or parallel to the opening of the aspiration catheter may result in an opening (e.g., a gap or space) into the aspiration lumen between the side(s) of the occluder or disruptor and an inner wall of the aspiration opening moving around the aspiration opening. In some cases, e.g., where the occluder or disruptor has a circular profile, such as when the occluder or disruptor is a disk, the opening may be a crescent-shaped opening that processes around the inner perimeter of the opening into the aspiration lumen as the occluder or disruptor is rotated and/or revolved. In general, clot material may be aspirated into the aspiration lumen though this moving opening, particularly after the region of the clot near the distal-facing end of the occluder or disruptor applies shear and thins the clot material. This may result in a dynamic lateral compression (e.g., squishing) of the clot material as it is drawn into the aspiration lumen, beneficially preventing the clot from clogging, in part by preventing fibrin from the clot from entangling with the occluder or disruptor or drive.

In any of these apparatuses and methods, moving the occluder or disruptor in the plane may include rotating a drive cable that is eccentrically coupled to the occluder or disruptor. The drive cable may be part of a drive or transmission that extends through the aspiration lumen. The suction may be started before moving the occluder or disruptor.

In any of these methods, suction (e.g., the application of negative pressure, also referred to as aspiration) may be started after the occluder or disruptor is driven to move (e.g., orbit) about the plane parallel to the aspiration opening. As mentioned, any of these methods may include contacting the clot material with a distal face of the occluder or disruptor prior to moving the occluder or disruptor in the plane that is parallel to the opening of the aspiration catheter. Without being bound by theory, this may thin the clot by shear thinning due to the rotating and/or revolving the distal face of the occluder or disruptor against the clot material, allowing it to be drawn in through the moving opening between the wall of the lumen and the outer edge(s) of the occluder or disruptor, while at the same time applying dynamic lateral compression. This may allow the rapid aspiration of even harder clot material, such as clot material having a high fibrin content.

For example, a method of removing a clot material may include: positioning an aspiration catheter comprising an aspiration lumen so that an opening into the aspiration lumen is proximate to a clot material; rotating a drive shaft to eccentrically revolve an occluder or disruptor in a plane that is parallel to the opening of the aspiration catheter in an orbital manner so that an opening into the aspiration lumen between the occluder or disruptor and an inner wall of the aspiration opening moves around the aspiration opening; and applying suction while moving the occluder or disruptor to draw the clot material through the moving opening into the aspiration lumen between the occluder or disruptor and an inner wall of the aspiration opening.

In some cases a method of removing a clot material may include: positioning an aspiration catheter comprising an aspiration lumen so that an opening into the aspiration lumen is proximate to a clot material; revolving an occluder or disruptor in a plane that is parallel to the opening of the aspiration catheter in an eccentric manner so that an opening into the aspiration lumen between an outer edge of the occluder or disruptor and an inner wall of the aspiration opening revolves around the aspiration opening; and applying suction while moving the occluder or disruptor to draw clot material through the moving opening into the aspiration lumen.

Any of the methods described herein may be methods in which the occluder or disruptor moves in an orbital manner around and/or across the plane of the opening (or an adjacent plane that is parallel or substantially parallel to the opening).

For example, described herein are methods (e.g., for removing a clot) that include: positioning an aspiration catheter so that an opening into an aspiration lumen through the aspiration catheter is proximate to a clot material; and eccentrically revolving a disc-shaped occluder or disruptor transversely around an opening into the aspiration lumen so that a distal face of the disc-shaped occluder or disruptor moves orbitally across the opening, while applying suction to draw clot material past the orbitally moving distal face and through the opening.

A method may include: positioning an aspiration catheter so that an opening into an aspiration lumen of the aspiration catheter is proximate to a clot material; dynamically changing the shape of the opening into the aspiration lumen by eccentrically revolving an occluder or disruptor transversely across the passage; and applying suction to draw clot material through the opening and into the aspiration lumen.

In some examples a method may include: positioning an aspiration catheter so that an opening into an aspiration lumen of the aspiration catheter is proximate to a clot material; and eccentrically revolving a disc-shaped occluder or disruptor transversely around an opening into the aspiration lumen by continuously accelerating, decelerating and reversing the rotation of a drive shaft coupled to the disc-shaped occluder or disruptor; and applying suction to draw the clot material against the disc-shaped occluder or disruptor and into the aspiration lumen.

In some cases it may be particularly beneficial to engage the occluder or disruptor at or near the distal end opening after the catheter has been positioned adjacent to or otherwise near (or in/against) the clot to be removed. For example, a method of removing clot material may include: positioning a catheter within a vessel; advancing an occluder or disruptor assembly within a lumen of the catheter until the occluder or disruptor assembly engages an annular guide assembly within a distal end region of the lumen so that an occluder or disruptor at a distal end of the occluder or disruptor assembly is positioned eccentrically across an opening into the lumen of the catheter to occlude a portion of the opening that is offset from a center of the opening; and rotating a drive shaft to eccentrically revolve the occluder or disruptor transversely around the opening while applying aspiration through the lumen.

Any of these methods may include advancing the catheter against a clot material to aspirate the clot material into the lumen of the catheter while rotating the drive shaft to dynamically compress the clot material between an edge of the revolving occluder or disruptor and an inner diameter of the opening. In any of the methods described herein, the method may also include advancing the catheter against a clot material to alter the viscosity of the clot material and aspirate the clot material into the lumen of the catheter.

As mentioned, the occluder or disruptor may be rotated by rotating a drive shaft. In any of these methods and apparatuses the drive shaft may rotate within a sleeve (e.g., a cover, sheet, etc.). Thus, the drive shaft may rotate relative to the sleeve, which may be coupled to the catheter or a component coupled to the catheter to limit or prevent rotation of the sleeve, while allowing the drive shaft to rotate freely within the sleeve. Thus, for example, any of these methods may include rotating the drive shaft within a sleeve extending through the lumen of the catheter.

In general, the occluder or disruptor may be part of an occluder or disruptor assembly that is mounted to (and rotated by) the drive shaft. The occluder or disruptor assembly may generally include one or more elements that help movably retain the occluder or disruptor assembly in the annular guide assembly within the distal end region of the lumen of the aspiration catheter. This retention element may be generically described as a retention protrusion or retention ring. The retention element may be a partial or full ring. In some cases the retention element comprises a retention protrusion that extends laterally from the occluder or disruptor assembly in a region behind (e.g., proximal to) the occluder or disruptor that is configured to extend perpendicularly across the aspiration opening. Thus, an occluder or disruptor assembly may engage an annular guide assembly by engaging a retention protrusion of the occluder or disruptor assembly within the annular guide assembly.

The annular guide assembly may be, for example, between about 1 mm and 5 cm of a distal end of the lumen (e.g., between about 1 mm and 4.5 cm, between about 1 mm and 4 cm, between about 1 mm and 3.5 cm, between about 1 mm and 3 cm, between about 1 mm and 2.5 cm, between about 1 mm and 2 cm, between about 1 mm and 1.5 cm, between about 1 mm and 1 cm, etc.).

As mentioned above, prior to removal of clot material, the occluder or disruptor assembly may be positioned eccentrically across the opening into the lumen of the catheter to occlude between about 30%-95% of the opening (e.g., between about 45%-95%, about 50%-95%, about 55%-95%, about 60%-95%, about 65%-95%, about 70%-95%, about 35%-90%, about 35%-90%, about 40%-90%, about 45%-90%, etc.).

Any of these methods may include moving (e.g., rotating and/or revolving) the occluder or disruptor by rotating the drive shaft, and in some cases rotating the drive shaft within the optional sleeve) at a rate that is between about 250 revolutions per minute (RPM) and 7000 RPM (e.g., between about 500 RPM and 7000 RPM, between about 500 RPM and 6000 RPM, between about 500 RPM and 5000 RPM, between about 750 RPM and 5000 RPM, between about 1000 RPM and 7000 RPM, between about 1000 RPM and 6000 RPM, between about 1000 RPM and 5000 RPM, etc.).

As mentioned above, it may be preferable to change the direction of the rotation. For example, any of these apparatuses may be configured to change between clockwise and counterclockwise, either instantaneously or with a pause or delay between changes in direction (e.g., between 0 and 5 seconds, between 1 ms and 5 seconds, between 1 ms and 2 seconds, between 1 ms and 1 second, etc.). Thus, any of these methods may include rotating the drive shaft by alternating the direction of rotation. Any of these methods may include increasing and/or decreasing the rate of rotation during the treatment. For example, the method may include continuously increasing and decreasing the rate of rotation.

Any of these methods may include advancing the catheter to, or adjacent and/or into the clot before or after the occluder or disruptor is positioned eccentrically across the opening and against a clot material.

For example, a method may include: positioning a catheter within a vessel; advancing an occluder or disruptor assembly within a lumen of the catheter until a retention protrusion of the occluder or disruptor assembly engages an annular guide assembly that is between 1 mm and 5 cm of a distal end of the lumen so that an occluder or disruptor at a distal end of the occluder or disruptor assembly is positioned eccentrically across an opening into the lumen of the catheter to occlude a portion of the opening; rotating a drive shaft to eccentrically revolve the occluder or disruptor transversely around the opening while retaining the retention protrusion within the annular guide assembly; and applying aspiration through the lumen. The method may include advancing the catheter against a clot material to aspirate the clot material into the lumen of the catheter while rotating the drive shaft to dynamically compress the clot material between an edge of the revolving occluder or disruptor and an inner diameter of the opening.

In any of these methods, method may include advancing the catheter against a clot material to alter the viscosity of the clot material and aspirate the clot material into the lumen of the catheter.

As mentioned, rotating the drive shaft may comprise rotating the drive shaft within a sleeve extending through the lumen of the catheter. Engaging the retention protrusion within the annular guide assembly may include engaging one or more of: a retention ring forming the retention protrusion, a split ring forming the retention protrusion, one or more wings forming the retention protrusion. In some cases engaging the retention protrusion within the annular guide assembly comprises engaging the retention protrusion against or between one or more of: a ring forming the annular guide assembly, a pair of rings forming the annular guide assembly, a channel forming the annular guide assembly.

The occluder or disruptor assembly may be positioned eccentrically across the opening into the lumen of the catheter to occlude, for example, between 30-95% of the opening. As mentioned above, rotating the drive shaft may comprise rotating the drive shaft between about 500 and 7000 RPM. In any of these examples rotating the drive shaft comprises alternating the direction of rotation.

For example, a method may include: positioning a catheter within a vessel; advancing an occluder or disruptor assembly within a lumen of the catheter until a retention protrusion of the occluder or disruptor assembly engages an annular guide assembly within a distal end region of the lumen so that an occluder or disruptor at a distal end of the occluder or disruptor assembly is positioned eccentrically across an opening into the lumen of the catheter to occlude between 30-95% of the opening; rotating a drive shaft to eccentrically revolve the occluder or disruptor transversely around the opening while retaining the retention protrusion within the annular guide assembly; and applying aspiration through the lumen.

Also described herein are systems including an occluder or disruptor assembly either with or without a catheter (e.g., an aspiration catheter). A system may include a catheter and occluder or disruptor assembly may include. For example, a system may include: a catheter having a lumen extending therethrough, an opening into the lumen, and an annular guide assembly within a distal end region of the lumen; and an occluder or disruptor assembly configured to be positioned within the catheter, the occluder or disruptor assembly comprising: an occluder or disruptor at a distal end of the occluder or disruptor assembly having a distal face that is configured to partially occlude the opening into the lumen of the catheter; a retention protrusion proximal to the occluder or disruptor and configured to movably engage the annular guide assembly of the catheter to limit axial movement of the occluder or disruptor assembly in the lumen; an elongated drive cable eccentrically coupled to the occluder or disruptor and configured to be rotated to drive revolution of the occluder or disruptor across the opening into the lumen of the catheter when the retention protrusion is engaged with the annular guide assembly of the catheter.

In any of the apparatuses described herein, the catheter may include an annular guide assembly with one or more annular rings. Annular guide assembly may project into the aspiration lumen and may be continuous or non-continuous (e.g., a series of protrusions forming a ring). In some examples the annular guide assembly may project into the lumen; alternatively, in some examples the annular guide assembly may be recessed into the wall of the lumen. The annular guide assembly may be located within the distal end region of the catheter, e.g., between about 2 mm and 5 cm from a distal end of the catheter.

In any of the apparatuses (and methods) described herein the occluder or disruptor assembly may include an occluder or disruptor that is coupled to an extender (e.g., dog leg extension) that links the occluder or disruptor assembly to the retention protrusion (e.g., retention ring) and may be coupled to the drive cable. The extender may be configured as a dog leg member.

The occluder or disruptor may be any of the occluders or disruptors described herein. In some cases the distal face of the occluder or disruptor may be configured to occlude between, e.g., 30%-95% of the opening into the lumen of the catheter when the retention protrusion is engaged with the annular guide assembly. For example, the occluder or disruptor may be a disc-shaped occluder or disruptor. In some cases the distal face of the occluder or disruptor comprises a convex surface. The distal face of the occluder or disruptor may comprise a substantially flat surface. The retention protrusion may be positioned between about 1 mm and 5 cm from the occluder or disruptor. The retention protrusion may comprise one or more of: a retention ring, a one or more wings or arms, a split ring, etc.

Any of these apparatuses may include a sleeve extending over at least a proximal length of the drive cable, wherein the drive cable is configured to rotate relative to the sleeve. Any of these apparatuses may include a proximal drive connector at a distal end of the drive cable configured to connect to a rotary driver.

In general, the occluder or disruptor assembly may be configured to be removably engaged with the annular guide assembly by coupling/uncoupling one or more retention protrusions extending laterally from the occluder or disruptor assembly into the annular guide assembly. For example, a retention protrusion of the occluder or disruptor assembly may be removably coupled to the annular guide assembly. In some cases the retention protrusion comprises a ring (or a split ring or partial ring) that may be elastically deformed to fit into the annular guide assembly allowing the occluder or disruptor assembly to be precisely rotated around and across the distal opening into the aspiration lumen.

A system may include: a catheter having a lumen extending therethrough, an opening into the lumen, and an annular guide assembly within a distal end region of the lumen; and an occluder or disruptor assembly configured to be positioned within the catheter, the occluder or disruptor assembly comprising: an occluder or disruptor at a distal end of the occluder or disruptor assembly having a distal face that is configured to occlude between 30%-95% of the opening into the lumen of the catheter; a retention protrusion proximal to positioned between 1 mm and 5 cm from the occluder or disruptor and configured to slidably engage with the annular guide assembly of the catheter; an elongate drive cable eccentrically coupled to the occluder or disruptor and configured to be rotated to drive revolution of the occluder or disruptor orbitally within the opening into the lumen of the catheter when the retention protrusion is engaged with the annular guide assembly of the catheter.

For example, a system may include: a catheter having a lumen extending therethrough, an opening into the lumen, and an annular guide assembly within a distal end region of the lumen; an occluder or disruptor assembly configured to be positioned within the catheter, the occluder or disruptor assembly comprising: an occluder or disruptor at a distal end of the occluder or disruptor assembly having a disc-shaped distal face configured to partially occlude the opening into the lumen; a retention protrusion positioned between 1 mm and 5 cm from the occluder or disruptor and configured to engage with the annular guide assembly within the lumen of the catheter; an elongate drive cable eccentrically coupled to the occluder or disruptor and configured to be rotated to drive revolution of the occluder or disruptor eccentrically about the opening into the lumen when the retention protrusion is engaged with the annular guide assembly of the catheter.

Also described herein are apparatuses including an occluder assembly that may be provided separately from a catheter. For example, described herein are occluder or disruptor assembly apparatuses configured to be positioned within an aspiration catheter to remove clot, the apparatus comprising: an occluder or disruptor at a distal end of the occluder or disruptor assembly having a distal face that is configured to partially occlude an opening into a lumen of the catheter; a retention protrusion proximal to the occluder or disruptor and configured to movably engage an annular guide assembly within the catheter lumen to limit axial movement of the occluder or disruptor assembly in the lumen; and an elongated drive cable eccentrically coupled to the occluder or disruptor and configured to be rotated to drive revolution of the occluder or disruptor across the opening into the lumen of the catheter when the retention protrusion is engaged with the annular guide assembly of the catheter.

The occluder or disruptor may be coupled to the retention protrusion and/or the drive cable through a connector, such as a dog leg connector. For example, the occluder or disruptor is coupled to the retention protrusion by a dog leg member. The distal face of the occluder or disruptor may be configured to occlude between about 30%-95% (e.g., between about 45%-95%, about 50%-95%, about 55%-95%, about 60%-95%, about 65%-95%, about 70%-95%, about 35%-90%, about 35%-90%, about 40%-90%, about 45%-90%, etc.) of the opening into the lumen of the catheter when the retention protrusion is engaged with the annular guide assembly.

Any appropriate occluder or disruptor may be used. For example, the occluder or disruptor may comprise a disc-shaped occluder or disruptor. The distal face of the occluder or disruptor may comprise a convex surface. In some cases the distal face of the occluder or disruptor comprises a substantially flat surface. The retention protrusion may be positioned between about 1 mm and 5 cm from the occluder or disruptor. The retention protrusion may comprise one or more of: a retention ring, one or more wings or arms, a split ring, etc. The retention projection(s) may generally form a ring (even if the actual projection(s) does/do not form a complete ring. In some cases either the retention protrusion(s) and/or the annular guide assembly may be lubricated or may include a lubricous material to more freely allow sliding within of the retention protrusion(s) within the annular guide assembly.

Any of these apparatuses may include a sleeve extending over at least a proximal length of the drive cable, wherein the drive cable is configured to rotate relative to the sleeve. Any of these apparatuses may include a proximal drive connector at a distal end of the drive cable configured to connect to a rotary driver.

For example, an occluder or disruptor assembly apparatus configured to be positioned within an aspiration catheter to remove clot may include: a disc-shaped occluder or disruptor at a distal end of the occluder or disruptor assembly having a distal face that is configured to partially occlude an opening into a lumen of the catheter; a retention protrusion proximal to the occluder or disruptor and configured to movably engage an annular guide assembly within the catheter lumen to limit axial movement of the occluder or disruptor assembly in the lumen; a connector connecting the disc-shaped occluder or disruptor to the retention protrusion so that the distal face is perpendicular to a long axis of the connector; and an elongated drive cable eccentrically coupled to the occluder or disruptor through the connector wherein the elongated drive cable is configured to be rotated to drive revolution of the occluder or disruptor across the opening into the lumen of the catheter when the retention protrusion is engaged with the annular guide assembly of the catheter.

For example, an occluder or disruptor assembly apparatus configured to be positioned within an aspiration catheter to remove clot may include: a disc-shaped occluder or disruptor at a distal end of the occluder or disruptor assembly having a distal face that is configured to partially occlude an opening into a lumen of the catheter; a retention protrusion proximal to the occluder or disruptor and configured to movably engage an annular guide assembly within the catheter lumen to limit axial movement of the occluder or disruptor assembly in the lumen; a connector connecting the disc-shaped occluder or disruptor to the retention protrusion so that the distal face is perpendicular to a long axis of the connector, wherein the connector extends between 2 mm and 5 cm; an elongated drive cable eccentrically coupled to the occluder or disruptor through the connector wherein the elongated drive cable is configured to be rotated to drive revolution of the occluder or disruptor across the opening into the lumen of the catheter when the retention protrusion is engaged with the annular guide assembly of the catheter; and a sleeve extending over at least a proximal length of the drive cable, wherein the drive cable is configured to rotate relative to the sleeve.

Also described herein are drivers for driving any of these occluder or disruptors. In general, the drivers may be hand-held drivers for driving rotation of an occluder or disruptor, e.g., by engaging with a proximal end of a drive transmission, such as a drive shaft, that is coupled to the occluder or disruptor to drive rotation. This engagement may be releasable.

For example, a hand-held rotary driver device may include: a drive motor; a housing enclosing the drive motor; a drive shaft coupled to the drive motor and extending distally; a distal drive connector on a distal end of the drive shaft, wherein the distal drive connector is configured to engage a proximal drive connector of an occluder or disruptor assembly apparatus; and a controller coupled to the drive motor and configured to drive rotation of the drive shaft at between 500 revolutions per minute (RPM) and 7000 RPM, wherein the controller is configured to alternate rotation of the drive shaft clockwise and counterclockwise.

The controller may generally be configured to control operation of the apparatus, including continuously accelerating and decelerating the rotation of the drive shaft. In some cases the controller is configured to cause the drive shaft to alternately rotate clockwise for a first duration, pause, and rotate counterclockwise for a second duration; the first duration, pause and second duration may each between about 0.1 second and 10 seconds. The distal drive connector may be configured to engage the proximal drive connector of an occluder or disruptor assembly apparatus through a keyed interface. The distal drive connector may include one or more magnets to engage the proximal drive connector of an occluder or disruptor assembly apparatus.

Any of the driver described herein may be configured to emit an audible indicator, e.g., during operation, such as when the apparatus is reversing direction (in variations that reverse direction), every x rotations (e.g., where x is between 1 and 3000, 1 and 2000, 1 and 1500, 1 and 1000, 1 and 500, etc.) within the housing that is configured to emit a sound indicating operation of the drive motor. For example, the audible indicator may comprise a clacker (e.g., an element that produces a clacking noise) configured to emit a sound indicating a change in rotational direction of the drive motor. In any of these apparatuses (systems, means, etc.) housing may be configured as a handle. For example, the apparatus may be configured to emit an audible indicator within the housing that is configured to emit a sound indicating operation of the drive motor. In some cases the audible indicator comprises a clacker configured to emit a sound indicating a change in rotational direction of the drive motor.

In general, any of the drive devices described herein may be hand-held rotary devices further comprising a control to activate driving of the drive shaft. For example, a hand-held rotary driver device may include: a drive motor; a housing enclosing the drive motor, wherein the housing is configured as a handle; a drive shaft coupled to the drive motor and extending distally; a distal drive connector on a distal end of the drive shaft, wherein the distal drive connector comprises a mechanical interlock configured to engage a proximal drive connector of an occluder or disruptor assembly apparatus; and a controller coupled to the drive motor and configured to drive rotation of the drive shaft at between 500 revolutions per minute (RPM) and 7000 RPM, wherein the controller is configured to alternate rotation of the drive shaft clockwise and counterclockwise and to continuously accelerate and decelerate the rotation of a drive shaft.

The mechanical interlock may include one or more keyed projections configured to engage with a corresponding one or more recesses on the proximal drive connector.

The controller may be configured to cause the drive shaft to alternately rotate clockwise for a first duration, pause for a second duration, and rotate counterclockwise for a third duration. For example, the first duration, second duration and third duration may each be between about 0.1 second and 10 seconds. In some cases, at least two of the first duration, the second duration and the third duration may have different lengths of time.

For example, the drive shaft connector may be configured to include one or more magnets to engage the proximal drive connector, e.g., of an occluder or disruptor assembly apparatus.

A hand-held rotary driver device may include: a drive motor; a housing enclosing the drive motor, wherein the housing is configured as a handle; a drive shaft coupled to the drive motor and extending distally; a distal drive connector on a distal end of the drive shaft, wherein the distal drive connector comprises a mechanical interlock configured to engage a proximal drive connector of an occluder or disruptor assembly apparatus; and a controller coupled to the drive motor and configured to drive rotation of the drive shaft at between 500 revolutions per minute (RPM) and 7000 RPM, wherein the controller is configured to cause the drive shaft to alternately rotate clockwise for a first duration, pause, and rotate counterclockwise for a second duration, wherein the first duration, pause and second duration are each between about 0.1 second and 10 seconds.

All of the methods and apparatuses described herein, in any combination, are herein contemplated and can be used to achieve the benefits as described herein.

### BRIEF DESCRIPTION OF THE DRAWINGS

FIG. 1A is a perspective view of a clot extraction catheter having an enlarged distal end with portions broken away to expose a clot-disruption structure with an occluder or disruptor or other clot-disruption element, a retention ring, and a distal stop band in accordance with the principles of the disclosed technology.

FIG. 1B is a perspective view of a clot extraction catheter similar to that of FIG. 1A, having a clot-disruption structure with an open, ring-like clot-disruption element.

FIG. 2 is detailed view of the distal region of the clot extraction catheter of FIG. 1A shown in partial section with the clot-disruption element fully advanced in the distal direction.

FIG. 3 is a view similar to that of FIG. 2 shown with the clot-disruption element revolved and rotated by 180°.

FIGS. 4A to 4C are distal end views of the clot extraction catheter of FIG. 1A shown with the clot-disruption element revolved and rotated in three positions separated by 120°.

FIG. 5A illustrates a drive cable assembly including a drive cable and an outer sleeve in accordance with the principles of the disclosed technology.

FIGS. 5B and 5C illustrate the relative rotation and revolution as the drive able is rotated.

FIG. 6 is a detailed view of the distal region of an alternative embodiment of a clot extraction catheter in accordance with the principles of the disclosed technology shown in partial section with an alternative clot-disruption element fully advanced in the distal direction.

FIG. 7 is a distal end view of the clot extraction catheter of FIG. 6.

FIG. 8 is a perspective view of an alternative embodiment of a clot-disruption structure of the disclosed technology having a C-shaped retention element.

FIGS. 9 and 10 are side and back views of the clot-disruptive structure of FIG. 8.

FIG. 11 is a perspective view of the clot-disruptive structure of FIGS. 8 to 10 shown with the C-shaped retention element disposed between distal and proximal stop bands in an open distal region of an aspiration catheter in accordance with the disclosed technologies.

FIGS. 12A and 12B are side, cross-sectional views of the open distal region of the aspiration catheter of FIG. 11 shown with the clot disruptive structure rotated in diametrically opposed positions.

FIG. 13 shows distal and proximal stop bands integrated into a single structure.

FIGS. 14A and 14B show the integrated stop band assembly of FIG. 13 positioned in an open distal region of an aspiration catheter with the clot-disruption structure of FIG. 11 being advanced (FIG. 14A) until the retention element is received in the integrated stop band assembly (FIG. 14B).

FIG. 15 illustrates a handle including a motor for use with the clot extraction catheters of the disclosed technology.

FIGS. 16A, 16B and 16C illustrate a handle assembly including a distal housing fixedly attached to the drive cable and a proximal housing for the motor, battery, and circuitry removably attached to the distal housing.

FIG. 17 is a detailed, perspective view of alternative embodiment of a clot-disruption structure including a disc-like clot-disruption element attached to a C-shaped retention element

FIG. 18 is a cross-sectional view of an annular guide located in an open distal region of a clot aspiration catheter with the clot-disruption structure of FIG. 17 shown in broken line.

FIG. 19 illustrates an alternative clot-disruption structure including a disc-like flow-blocking plate having an open passage formed therethrough to define an aspiration zone.

FIGS. 20A-20D illustrate alternative disc-like flow-blocking plate shaving open passages with different geometries.

FIG. 21 illustrates exemplary thrombectomy locations.

FIGS. 22A-22C illustrates an exemplary system for removing clot from a blood vessel, in accordance with various embodiments of the present disclosure.

FIGS. 23A-23C illustrate an exemplary clot disruptor assembly, in accordance with various embodiments of the present disclosure.

FIG. 24 illustrates an exemplary elongated drive cable, in accordance with various embodiments of the present disclosure.

FIG. 25 illustrates an exemplary a non-rotating sleeve, in accordance with various embodiments of the present disclosure.

FIGS. 26A-26B illustrate an exemplary aspiration catheter, in accordance with embodiments of the present disclosure.

FIG. 27 illustrates an exemplary system, in accordance with embodiments of the present disclosure.

FIG. 28 is a flowchart of a method for removing clot from a blood vessel, in accordance with embodiments of the present disclosure.

### DETAILED DESCRIPTION

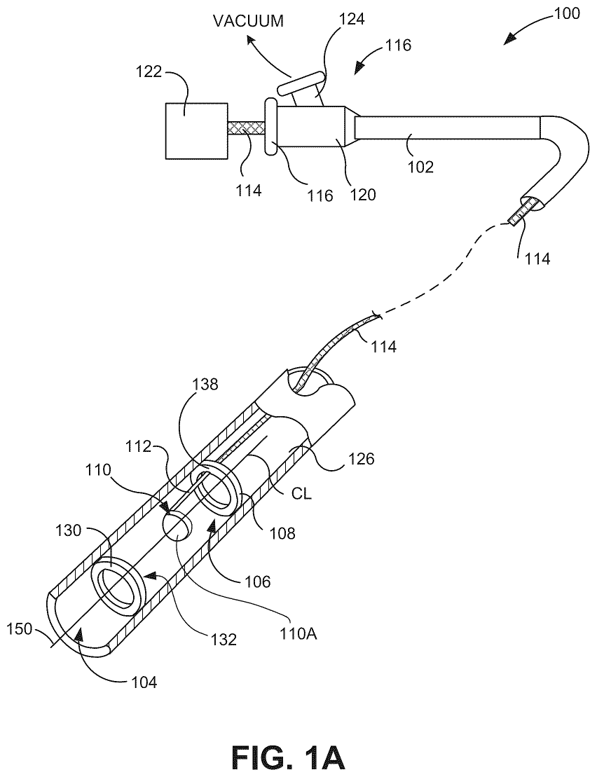

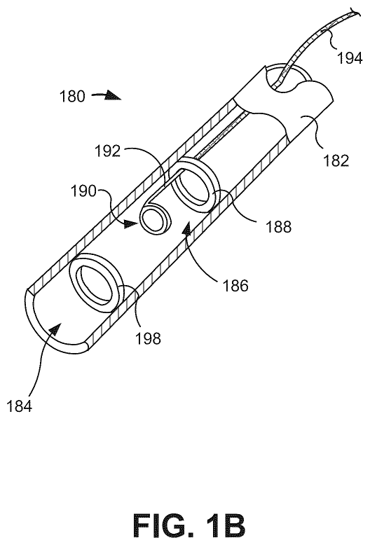

As shown in FIG. 1A, an exemplary clot extraction catheter 100 constructed in accordance with the disclosed technologies comprises a catheter body 102 having an open distal region 104 with portions broken away to expose a clot-disruption structure 106 including a retention ring 108 and a clot-disruption element 110 such as a disc-like clot-disruption element. The clot-disruption structure 106 is attached to a distal end of a drive cable 114, and the drive cable is configured to rotate and/or rotationally oscillate the clot-disruption structure about a central axis 150 of the open distal region. As explained in greater detail below, rotation of the clot-disruption structure 106 as a whole about the central axis 150 causes the clot-disruption element 110, which is radially offset from a center line CL of the clot distribution structure, to revolve about the center axis of the open distal region. As shown in FIG. 1A, the center line CL of the clot-disruption structure is coincident with the central axis 150 of the open distal region when the clot-disruption structure is in the open distal region.

A proximal end of the drive cable 114 extends through a proximal port 116 on a proximal hub 120 and is connected to a rotational drive motor 122 which can be controlled to rotate and/or rotationally oscillate the clot-disruption structure 106 at a rate in a range from between about 1000 RPM to 5000 RPM, inclusive, sometimes being about 3000 RPM, usually being about 4000 RPM. The drive cable 114 is usually configured to transmit torque in both clockwise and counterclockwise directions, typically comprising counter wound coils, often formed over a solid core wire.

In most embodiments, at least some of the operational parameters of the rotational drive motor 122 will be automatically controlled, for example to alternate rotation between clockwise and counterclockwise rotational directions. In some cases, the rotational drive motor 122 may be further configured to continuously accelerate or decelerate while reversing rotational directions. In some cases, the drive motor 122 and/or associated control circuitry is configured to rotate in a first rotational direction for a period in a range between about 0.1 second and 10 seconds, often from about 0.1 second to 5 seconds, from about 0.5 seconds to 5 seconds, from about 0.5 seconds to 2 seconds, from about 0.1 second to 2 seconds, and frequently from about 0.1 second to 1 second. Rotation may be paused for a short time period, usually from about 0.01 seconds to 5 seconds, often from about 0.05 seconds to 2 seconds, and more often from about 0.05 seconds to 1 second, and then after the pause, rotation may be started in the opposite rotational direction, although in some case, rotation could be resumed in the same rotational direction. The durations of clockwise and counterclockwise rotation may be the same or different, and the time period of a pause between clockwise-to-counterclockwise rotations may be shorter or longer than that of a pause between the clockwise-to-counterclockwise, or they may be the same.

In some cases, the drive motor 122 and/or associated control circuitry may be configured to automatically switch between clockwise and counterclockwise directions without pausing. In some cases, the drive motor 122 may be configured to pause or hold for a fixed or variable amount of time when switching between clockwise/counterclockwise rotation. For example, the hold time may be in a range between about 0.05 second and about 1 second, about 0.1 second and about 0.5 second, about 0.1 second and about 0.3 second, about 0.05 second and about 0.3 second, about 0.1 second, about 0.2 seconds, about 0.3 seconds, about 0.4 seconds, about 0.5 second, and the like.

In any of the methods and apparatuses described herein, the drive motor 122 may be configured to rotate the drive cable 114 continuously or near continuously, e.g., in a state of either acceleration or deceleration, or optionally with a pause, e.g., of approximately 0.5 seconds therebetween.

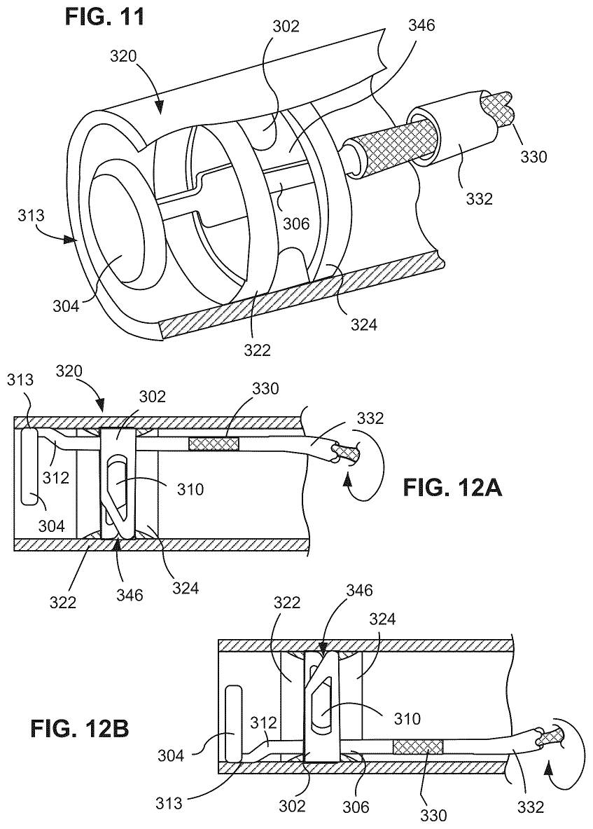

In any of the methods and apparatuses described herein, the drive motor 122 may be configured to rotate the drive cable 114 and thus the clot-disruption element 110 at a slow speed, for example between 1 RPM and 100 RPM, 5 RPM and 80 RPM, usually being about 50 RPM, either in a single direction, or in alternating directions for brief durations, e.g., several seconds, to facilitate the advancement of the clot-disrupting element 304, e.g., the C-shaped retention ring or other structure as it is advanced into position distal to the proximal stop band 324, as described with reference to FIGS. 11, 12A and 12B, below. The slow rotation has been observed to overcome sliding friction between the C-shaped retention structure and the proximal retention ring.

In some cases, at least some the operational parameters of the drive motor 122 can be controlled manually. For example, a user could control on-and-off operation of the drive motor 122 using a hand or foot switch. The user could also set rotational speed, reversal times, and the like, using manual controls connected to the drive motor 122 or to associated control circuitry.

A vacuum port 124 located on the proximal hub 120 can be connected to a vacuum source for aspirating clot or thrombus through clot-extraction lumen 126 which extends from the proximal hub 120 to the open distal region 104. Suitable vacuum sources include the Medela pump which is part of the AXS Universal® Aspiration Set available from Stryker Corporation, Kalamazoo, Michigan. The clot-disruption element 110 is prevented from being distally advanced beyond the open distal region 104 of the catheter body 102. A distal stop band 130 on the inner wall of the open distal region engages the retention ring 108 to limit distal advancement of the clot-disruption structure 106. A proximal bearing surface 132 of the distal stop band 130 engages a distal bearing surface 138 on the retention ring 108 as the clot-disruption structure 106 is distally advanced through the clot-extraction lumen 126 to prevent further distal advancement of the clot-disruption element 110.

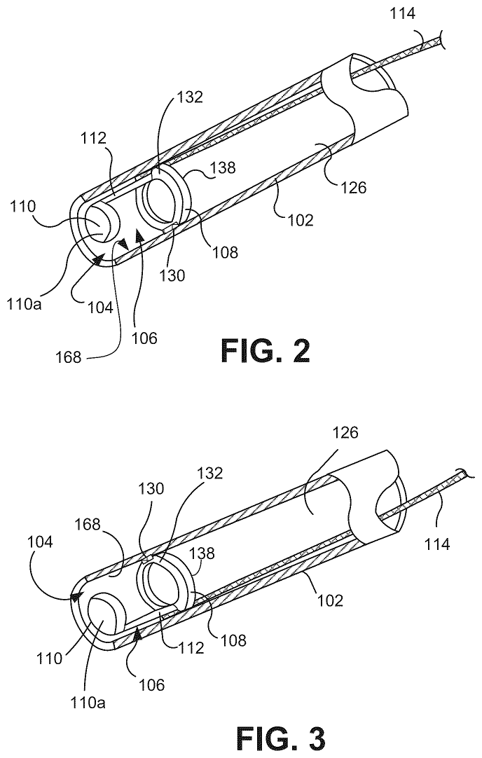

As shown in FIGS. 2 and 3, when the clot-disruption structure 106 is fully advanced in the distal direction, the disc-like clot-disruption element 110 is positioned at or close to a plane of an open end of the open distal region 104 (but will preferably not extend beyond that plane). When so located, the disc-like clot-disruption element 110 partially block the flow of the blood and clot into the clot-extraction lumen 126, diverting the blood and clot entering the open distal region 104 of the catheter body 102 to flow between an outer periphery of the clot-disruption element and an inner wall 168 of the open distal region 104 of the catheter body 102. The clot-disruption structure 106 is then rotated by the drive motor 122 and drive cable 114, causing the clot-disruption structure 106 to revolve, shown in its 12 o'clock position in FIG. 2 and it 6 o'clock position in FIG. 3.

The surface area of the distal-facing surface of the disc-like clot-disruption element 110 will typically be between about 25% and 95% of the cross-sectional area of the open distal region 104 of the catheter body 102 in which it rotates, often being in ranges between 30%-95%, between 40%-95%, between 50%-95%, between 60%-90%, between 30%-90%, between 50%-90%, between 40%-90%, between 50%-85%, between 40%-85%, and between 55%-90%.

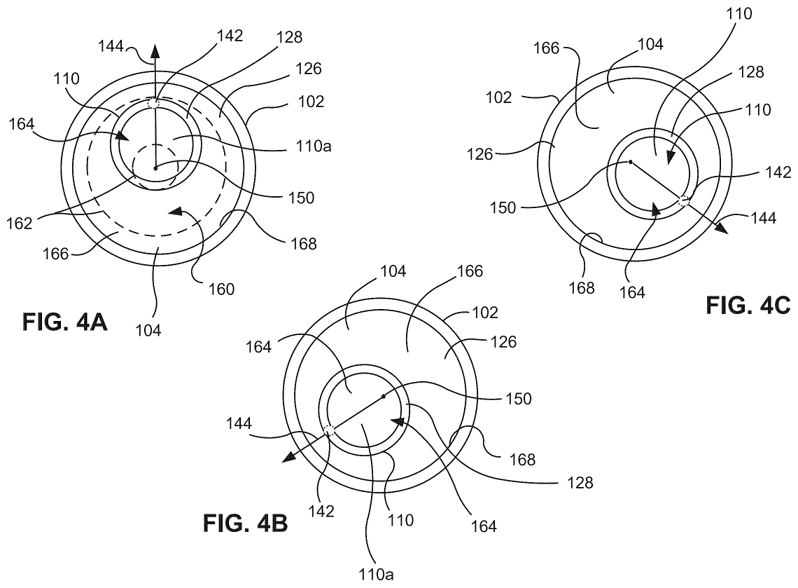

A distal-facing surface 110a of the solid clot-disruption element 110 is typically oriented in a plane perpendicular to the central axis 150 of the open distal region 104 of the clot-extraction lumen 126 so that it appears to cover or span a portion or percentage of the cross-sectional area of the open distal end, as best seen in FIGS. 4A to 4C.

The distal-facing surface 110a will usually be planar (flat and preferably free of features) and oriented orthogonally (at) 90° with respect to the central axis 150 of the open distal region 104. In other instances, however, the distal-facing surface may be non-planar (not illustrated), for example being curved, wavy, convex, or concave in the distal direction, or the like. When convex or concave, the distal facing surface will usually have a radius of curvature that is greater than a radius of the open distal region 104 of the clot-extraction lumen 126, for example having a radius of curvature which is 1.5-fold, 2-fold, 3-fold, 4-fold, 5-fold, 10-fold, or more, greater than the radius of the open distal region.

The distal facing surface 110a of the solid clot-disruption element 110 will also typically be smooth and free from irregularities, but in certain embodiments the surface may be textured, pitted, scored, porous, perforated, and in some cases may include surface features such as bumps, ridges, penetrating elements, cutting elements, and the like, formed over all or a portion of the distal-facing surface, which features may be integrated or attached.

In some instances the clot-disruption elements of the disclosed technologies can be partially or fully open in their centers, and in some cases comprise open ring structures which are asymmetrically mounted and revolved in the open distal region of the aspiration catheter. For example, as shown in FIG. 1B, an alternative clot extraction catheter 180 comprises a catheter body 182 having an open distal region 184 with portions broken away to expose a clot-disruption structure 186 including a retention ring 188 and an open, ring-like clot-disruption element 190. In all other respects, the structure of clot aspiration catheter 180 is similar to that of clot aspiration catheter 100, and catheter 180 includes a connecting strut 192, a drive cable 194, and a distal stop band 198.

The clot-disruption structure 106 will typically be centered within the open distal region 104, while the clot-disruption element 110 will be radially offset to allow the clot-disruption element to revolve about the central axis 150 while the clot-disruption structure as a whole is rotated within the open distal region. As shown in FIGS. 1-3, the connecting strut 112 is straight and spaced radially inwardly from the inner wall 168 as to enable the strut to clear the distal stop band 130. In other embodiments, the clot-disruption element 110 could be moved radially inwardly or outwardly by bending or otherwise reshaping the connecting strut 112, for example, as shown in FIGS. 10A to 14B and associated text below.

The length of the connecting strut may vary widely but typically will be in a range from about 0.4 mm to about 5 mm, often from about 0.5 mm to 3 mm, from about 0.75 mm to 2.5 mm, from about 0.8 mm to about 2.3 mm, from about 0.5 mm to 1.5 mm, and the like.

The axial strut will typically be configured to allow a peripheral location on the clot-disruption element to closely follow or track the inner wall of the open distal region while the clot-disruption element revolves in the open distal region. See FIGS. 10A to 14B and associated text below.

Referring again to FIG. 1A, the clot-disruption structure 106 is proximally retracted so that the retention ring 108 is positioned proximally to the distal stop band 130 which is fixed to an inner wall of the clot-extraction lumen 126. The position of the distal stop band 130 defines the size of the open distal region 104. A distal edge of the distal stop band 130 is typically spaced from 2 mm to 10 mm proximally of a plane of the distal opening of the catheter lumen, but the placement can vary significantly.

In use, the open distal region 104 of the clot extraction catheter 100 is intravascularly advanced to a location proximate a region of clot, typically in the neurovasculature. In such instances, the catheter 100 may be first advanced without the clot-disruption structure 106/drive cable 114 assembly, and the assembly then advanced through the clot-extraction lumen 126 until the distal bearing surface 138 of the retention ring 108 engages the proximal bearing surface 132 of the distal stop band 130, as shown in FIGS. 2 and 3. Alternatively, the apparatus may be inserted with the clot-disruption structure 106 pre-inserted into the catheter. The opposed bearing surfaces 132 and 138 prevent further distal advancement while allowing the retention ring 108 to be rotated against the distal stop band 130.

The retention ring 108 is rotated by the drive cable 114 which itself is rotated by the drive motor 122 (FIG. 1A). Rotation of the retention ring 108 in turn causes the connecting strut 112 to revolve about the central axis 150 of clot-extraction lumen 126, as seen in FIGS. 4A to 4C.

The revolving connecting strut 112 causes the clot-disruption element 110 to similarly revolve about the central axis 150, as can be seen by comparing FIGS. 2 and 3. While the clot-disruption element will also be rotating, it is the revolving motion that has been found to enhance the clot break-up and aspiration. Clot-disruption element 110, as illustrated in FIGS. 4A to 4C, comprises an outer frame 128 filled with gold or other radiopaque (RO) material, but could alternatively have any one of many structures as described elsewhere herein.

While the disclosed technologies can utilize a variety of different clot-disruption element designs, the use of disc-like and other flow-blocking structures has been found to be particularly effective. 388 have an outer diameter which is typically in a range from 25% to 95% of an inner diameter of the open distal region 104 of the clot-extraction lumen 126, often being in a range from 25% to 95% of the inner diameter of the open distal region of the clot-extraction lumen. While disc-like (circular) flow-blocking elements have been found particularly useful, other geometries including oval, polygonal, lobed, irregular, and the like, may also find use.

Revolution of the clot-disruption element 110 in the open distal region 104 of the catheter body 102 can be seen most clearly in FIGS. 4A to 4C. As shown in FIG. 4A, the clot-disruption element is at the 12 o'clock position with the strut connecting point 142 on directional vector 144. Rotation of the drive cable 114 (FIGS. 1-3), causes the clot-disruption element 110 to revolve and follow an annular path 160 delineated by broken lines 162. The annular path of travel is beneficial as it moves the disc-like or other clot-disruption element 110 through distinct clot passage zones defined by an open inner region 164 and an outer region 166 defined between the outer edge of the clot-disruption member and an inner wall 168 of the open distal region 104.

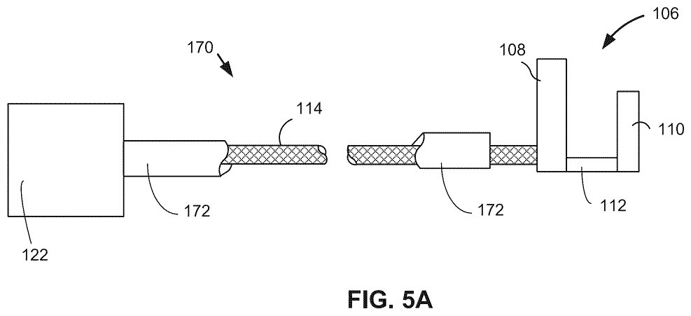

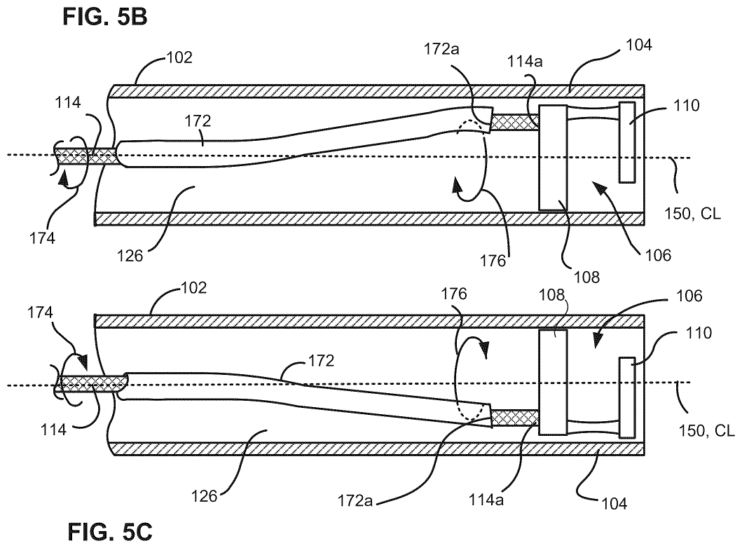

Referring now to FIG. 5A, in exemplary embodiments the drive cable 114 will be incorporated into a drive cable assembly 170 where the drive cable is disposed in an outer sleeve 172. In some cases, the sleeve will be fixed to the cable so that the cable and sleeve rotate together as they are driven by the drive motor 122. In most cases, the drive cable 114 will rotate within an inner lumen of the sleeve and the outer sleeve 172 will be configured not to rotate, as shown in FIGS. 5B and 5C. The later design may be advantageous as it reduces interference and friction between the rotating drive cable and the clot and luminal wall of the catheter as the clot is being aspirated.

As shown in FIGS. 5B and 5C, the drive cable 114 is rotated about its own axis, as indicated by arrow 174. Rotation of drive cable 114 causes the clot-disruption structure 106 to synchronously rotate with a distal end 114a of the drive cable following an orbital path as indicated by arrow 176. In contrast, the outer sleeve does not rotate about its own axis, but its distal end 172a does follow the same orbital path indicated by arrow 176. Thus, the clot entering the open distal region 104 is shielded from the rotation of the drive cable 114 which could wrap the fibrin content of the clot and risk clogging the clot-extraction lumen 126. The non-rotating sleeve also prevents any undesirable corkscrew movement and/or hydrodynamic vortices. In contrast, precession of the distal region of the outer sleeve 172 about the central axis 150 without rotation about its own axis can promote clot break up and enhance aspiration while minimizing fibrin entanglement.

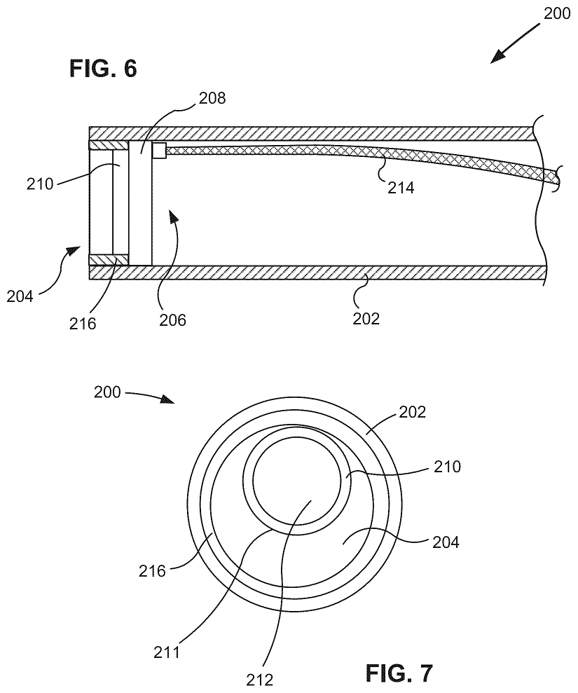

FIG. 6 is detailed view of the distal region of an alternative embodiment of a clot extraction catheter 200 constructed in accordance with the principles of the disclosed technology with an alternative clot-disruption structure 206 fully advanced in a distal direction in an open distal region 204 of a catheter body 202. In contrast to the clot-disruption structure 106, the clot-disruption structure 206 does not include a connecting strut so that a proximal side of a clot-disruption element 210 is fixed directly to a distal side of a retention ring 208.

As shown in FIG. 7, a distal stop band 216 is located at the distal tip of the open distal region 204 of so that the clot-disruption element 210 is located at or within a very short distance (e.g., 0 to 0.5 mm, often 0 to 0.1 mm) from a distal tip when the retention ring 208 is advanced fully distally against the distal stop band 216. A drive cable 214 is provided to rotate the retention ring 208 which in turn revolves the clot-disruption element 210 along a similar annular path as described previously with reference to 4A to 4C. The clot-disruption element 210 comprises an outer frame 211 filled with an RO material similar to clot-disruption element 110 as shown in FIGS. 4A to 4C

In still other embodiments similar to that shown in FIGS. 6 and 7, the distal stop band 216 could be located some distance proximally of the distal tip of the open distal region 204 in order to recess the clot-disruption element 210 a desired distance from the open distal tip.

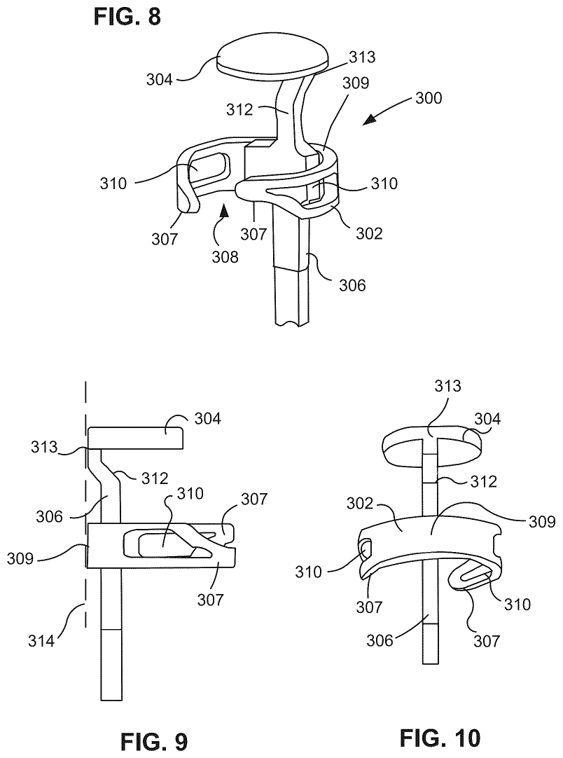

Referring now to FIGS. 8 to 10 an alternative clot-disruption structure 300 according to the disclosed technology comprises a C-shaped retention ring 302 and a disc-like clot-disruption element 304. The C-shaped retention ring 302 has two wings 307 separated by a gap 308. Each wing 307 is formed from an elastic material, such as a spring stainless steel or a shape memory alloy, such as a nickel-titanium alloy. Each wing 307 will usually have a cutouts 310 to enhance flexibility, and a bridging section 309 disposed between the wings is attached to a connecting strut 306.

In contrast to prior embodiments, the connecting strut 306 will have a dog leg 312 or other offset portion with aligns a connection point 313 on the clot-disrupting element 304 with the bridging section 309 of the retention ring 302 as indicated by line 314 in FIG. 10. Such alignment allows the connection point 313 to closely sweep the inner wall of the open distal region 320 of the aspiration catheter (FIG. 11). Such close sweeping by an edge of the clot-disrupting element 304 can enhance clot aspiration in at least some circumstances.

FIGS. 11, 12A and 12B illustrates the C-shaped retention ring 302 of the clot-disruptive structure 300 of FIGS. 8 to 10 disposed between distal and proximal stop bands 322 and 324 in the open distal region 320 of an aspiration catheter. The distal and proximal stop bands 322 and 324 are axially spaced-apart to form an annular guide channel 326 which rotationally receives the clot-disrupting element 304, e.g., the C-shaped retention ring. Unlike prior embodiments, the clot-disrupting element 304, e.g., the C-shaped retention ring, may radially compress as it is distally advanced past the proximal stop band 324. The elasticity provided by the C-shape and use of elastic materials allows such radial compression. Additionally, at least the proximal stop band 324 can have a ramped cross-section which facilitates radial compression as the retention ring 302 is advanced. The distal stop band 322 may also have a ramped cross section to facilitate retraction should the retention ring 302 be accidentally advanced beyond the distal stop band. Also, in some instances, the distal stop band 322 may be higher than the proximal stop band 324 to lessen the barrier to entry of the retention ring 302 into the annular guide channel 346 while increasing the barrier which prevents further advancement past the distal stop band.

The connecting strut 306 will usually be connected to a drive cable 330 which is optionally located in an outer sleeve 332, as previously described with reference to FIG. 6. As the drive cable 330 is rotated, the retention ring 302 will be rotated in the annular guide channel 346 with the connecting struct causing the clot-disruption element 304 to revolve as can be seen by comparing the positions of the clot-disruption element in FIGS. 12A and 12B.

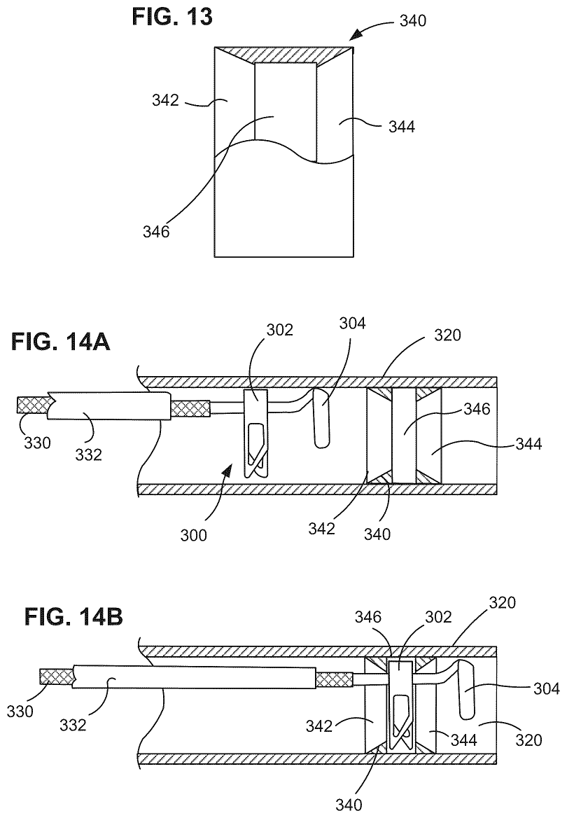

FIGS. 13, 14A, and 14B illustrate a stop band assembly 340 having distal and proximal stop bands 342 and 344, respectively, integrated into a single structure. The distal and proximal stop bands 342 and 344 define an annular guide channel 346 therebetween. One or more of the stop bands having distal and proximal stop bands 342 and 344 may include a beveled edge for assisting the retention ring 302 to slide between the distal and proximal stop bands 342 and 344 and maintain the retention ring 302 between the distal and proximal stop bands 342 and 344 during operation. The bevel may be formed via fine machining in some embodiments. During operation, the retention ring 302 may be rotated about 4000 rpm, thereby creating a dramatic increase in rotational velocity within the catheter lumen. The distal and proximal stop bands 342 and 344 and maintain the retention ring 302 in position during high velocity rotation. The integrated stop band assembly 340 assures the stop bands 342 and 344 will not fall out of alignment and disturb the annular guide channel 346 which could theoretically at least occur when the stop bands 322 and 324 are mounted separately on the inner wall of the open distal region 320, as shown in FIGS. 11, 12A and 12B.

As shown in FIGS. 14A and 14B, the clot-disruption structure 300 can be distally advanced in the open distal region 320 of an aspiration catheter so that the occluder or disruptor or other clot-disruption element 304 passes through both the proximal and distal stop bands 344 and 342 and the clot-disrupting element 304, e.g., the C-shaped retention ring, is captured in the annular guide channel 326 so that the axial position of the clot-disruption structure 300 is fixed (or proximal and distal axial movement of the clot-disruption structure 300 is limited) while the structure is free to rotate and cause the clot-disruption element 304 to revolve as previously described.

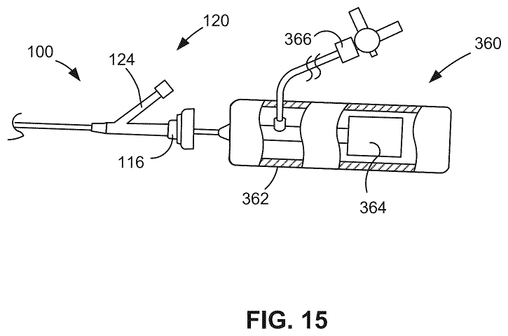

A first exemplary handle 360 configured for use with the aspiration catheters of the disclosed technology is illustrated in FIG. 15. The handle 360 comprises a housing 362 having a drive motor 364 therein and a flush port 366 for connecting to the proximal port 116 of aspiration catheter 100 (FIG. 1A). The drive motor 364 detachably connects to the drive cables 114, 170, 194, or 214, and the handle will typically be disposable.

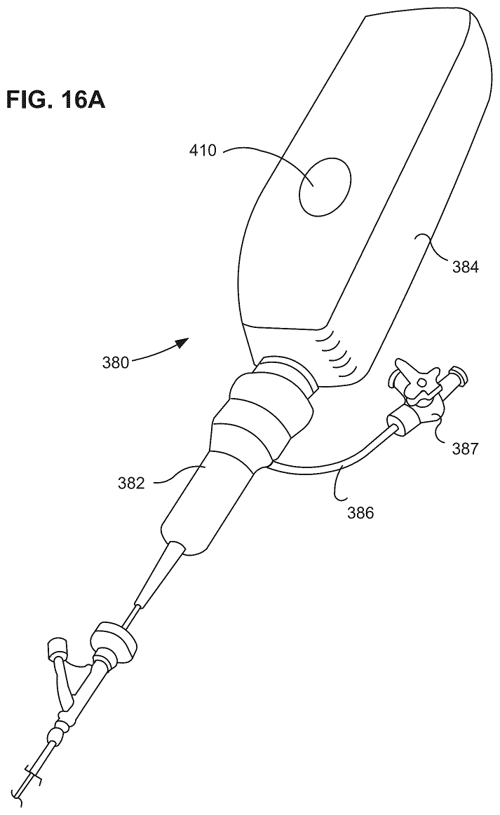

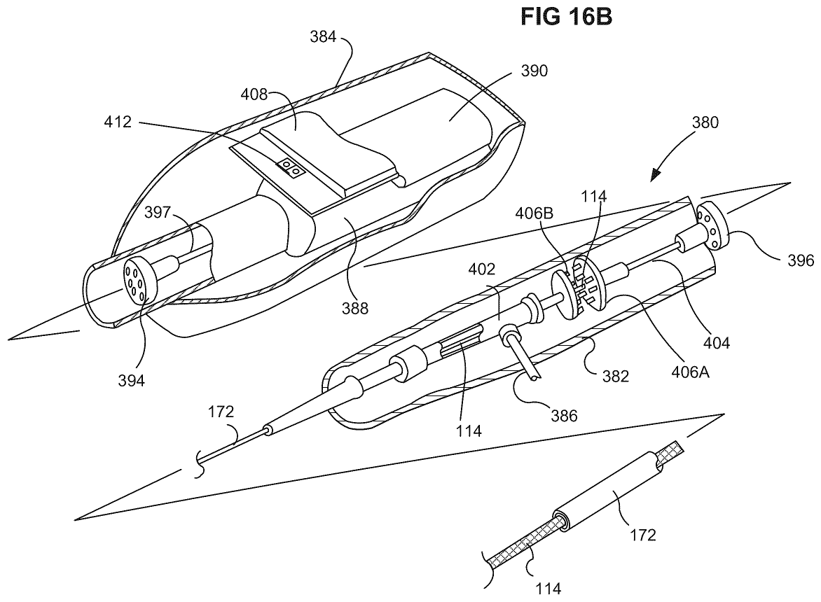

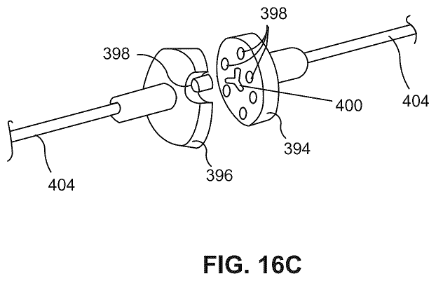

Referring now to FIGS. 16A, 16B, and 16C, a second exemplary handle assembly 380 configured for use with aspiration catheter 100 comprises a distal housing 382 and a proximal housing 384. The distal housing 382 includes a flush line 386 for providing a flushing fluid to the outer sleeve 172 positioned over the drive cable 114, as shown in FIG. 5A. The flush line has an external valve 387 for connecting to a fluid source and is internally connected to a tubular manifold 402 which supplies the fluid to a lumen of the outer sleeve 172 to flush the annular space between the drive cable 114 and an inner wall of the external sleeve. A proximal drive connector 396 (which is driven by motor 388 in proximal housing 384 as described below) rotates drive shaft 404 which is coupled to a proximal end of the drive cable 114. When connected to the motor, the drive cable 114 will be pushed distally causing clackers 406a and 406b to come into contact. Clacker 406b is stationary (drive cable 114 passes though it) while clacker 406a rotates with the drive shaft 414. Thus, the clackers 406a/b will engage and produce a sound when the motor 388 is rotating the drive cable 114, alerting that the clot-disruption structure is being rotated.

The clackers or other operational indicators are configured provide audible and/or tactile feedback to the user to indicate operation of the apparatus, specifically that the clot disruption structure and element are rotating and further provide an audible indication when the direction of rotation changes. The clackers operate mechanically to generate sound and/or vibration which the user can sense. Other suitable indicators may be electronic or electromechanical. For example, an electronic indicator may include a circuit configured to output a sound, light, vibration, or other indicator when the driver is rotating and/or the direction or speed of rotation has changed.

The proximal housing 384 includes a motor 388, battery 390, control circuitry 408, an external control 410 (such as one or more buttons), and internal control switches 412 actuated by the external control. The motor 388 rotates a drive shaft 397 which in turn rotates the proximal drive connector 394. As best seen in FIG. 16C, the proximal and distal drive connectors 394 and 396 are coupled by a magnetic coupling assembly including magnets 398 and a mechanical interlock 400 to prevent rotational slippage.

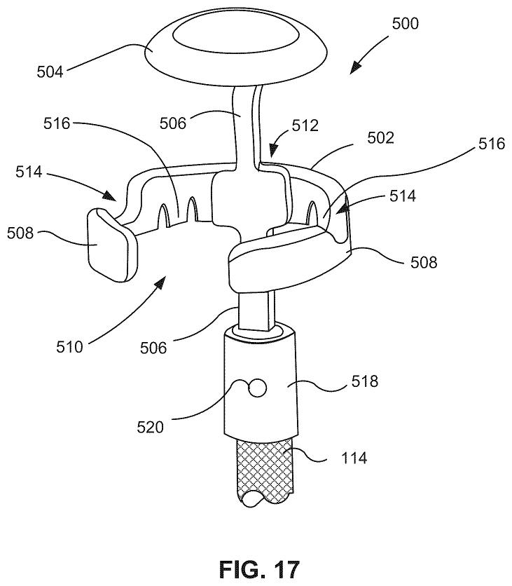

FIG. 17 illustrates a further alternative embodiment of a clot-disruption structure 500 including a disc-like occluder or disruptor attached to a C-shaped retention ring 504. The design is similar to that of clot-disruption structure 300 described with reference to FIGS. 8 to 10 and includes a connecting strut 506, a pair of wings 508, a gap 510 between distal tips of the wings, and bridging section 512 diametrically opposite the wings. In place of the cutouts 310 included in clot-disruption structure 300, wings 508 each include notches 514 which act as passive joints to allow the distal ends of the wings to bend radially inwardly as the clot-disruption structure 500 is advanced into any of the grooved retention structures described herein. The retention ring 502 is also provided with retention tabs 516 which project radially outwardly from the outer surface of the retention ring and act as stops to prevent the clot-disruption structure 500 from being accidentally displaced after having been positioned in the annular groove. The retention tabs are flared radially outwardly at an angle that allows them to depress radially inwardly when the retention ring 502 is advanced distally into an annular retention groove but spring back to inhibit withdrawal in a proximal direction.

FIG. 17 further illustrates connection of the clot-disruption structure 500 to the drive cable 114. A coupling sleeve 518 receive a distal end of the drive cable 114 and a proximal end of the connecting strut 506. The connecting sleeve 518 may be crushed, filled with an adhesive, solder, or other material, or combinations thereof, and the connection is usually completed with a fastening pin penetrating the sleeve 518, drive cable 114, and connection strut 506.

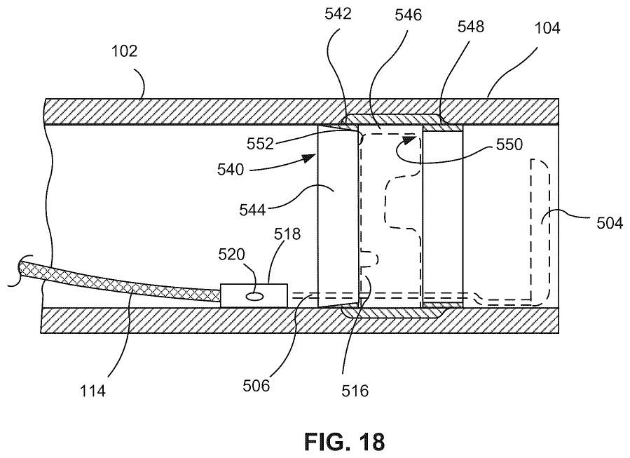

FIG. 18 is a cross-sectional view of an annular guide assembly 540 located in the open distal region 104 of catheter body 102 with the clot-disruption structure 500 of FIG. 17 shown in broken line. The annular guide assembly 540 comprises a proximal ring 542 having an inclined ramp 544 which tapers radially inwardly in the distal direction to facilitate entry of the retention ring 504 into an annular guide channel 546 as the clot-disruption structure 500 is distally advanced. A distal ring 548 may be formed separately from the proximal ring 542, for example being formed from a radiopaque (RO) material to facilitate fluoroscopic imaging. The proximal ring 542 defines a proximal stop surface 552 and the distal ring 548 defines a distal stop surface 550, and the stop surfaces together define the height or thickness of the annular guide channel 546 which will typically be chosen to be slightly larger than the height or thickness of the C-shaped retention ring 502 to allow the ring to rotate feely while maintaining the retention ring in the groove. The stop surfaces 550 and 552 will usually be oriented perpendicularly to the axis (i.e., not ramped) to improve retention of the retention ring 502 which will also have perpendicularly oriented surfaces on each side. The retention tabs 516 will deflect radially outwardly to enhance engagement with the proximal stop surface 552 to further assure that the clot-disruption structure 500 will not be accidentally pulled from the catheter during use.

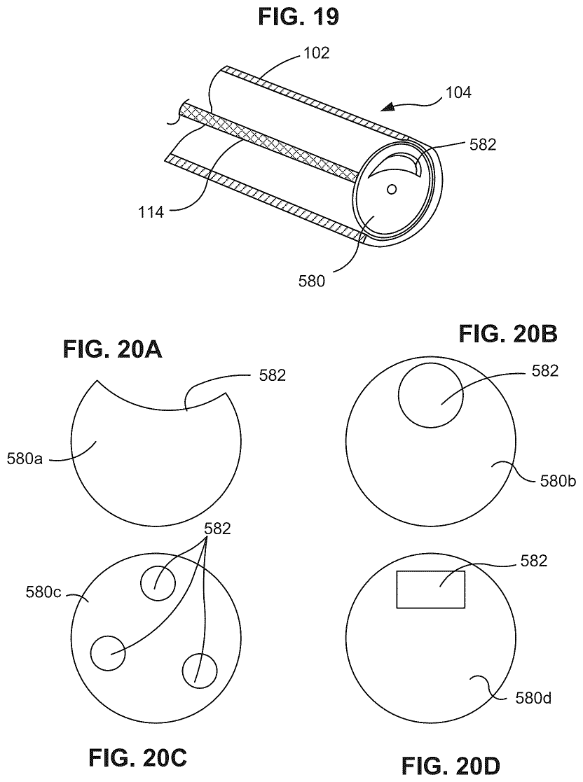

FIG. 19 illustrates an alternative clot-disruption structure 580 including a disc-like flow-blocking plate 580 having an open passage 582, e.g., a crescent shaped or other shaped passage, formed therethrough to define an aspiration zone. In contrast to previous embodiments, the disc-like flow-blocking plate 580 can be sized to block most or all of the open distal region 104 of catheter body 102. Flow of clot into the open distal region 104 occurs through the open passage 582 which can be rotated by rotating drive cable 114. The drive cable 114 is shown to be attached to a center of the disc-like flow-blocking plate 580, but it could also be asymmetrically attached as in any of the previously described embodiments. The drive cable 114 could also be positioned in an outer sleeve as previously described for other embodiments.

FIGS. 20A to 20D illustrate alternative passages formed in the rotating disc-like flow-blocking plate 580. For example, a disc-like flow-blocking plate 580a may have a crescent-shaped or other shaped cut out 581 in the periphery to define a limited area aspiration zone. A disc-like flow-blocking plate 580b may have a circular-shaped passage through the disc plate to define a limited area aspiration zone. A disc-like flow-blocking plate 580c may have a plurality of smaller circular-shaped passages through the disc plate to define the limited area aspiration zone, and a disc-like flow-blocking plate 580d may have a rectangular or other polygonal cut out through the disc plate to define the limited area aspiration zone.

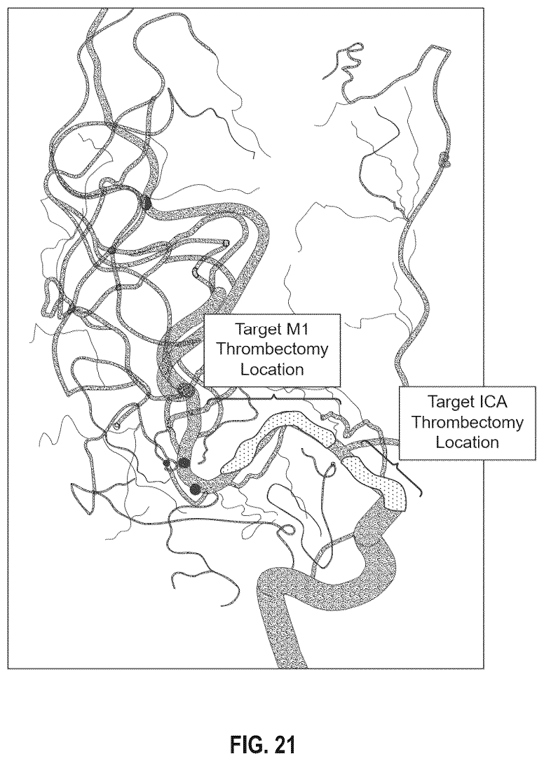

FIG. 21 illustrates exemplary thrombectomy locations. Thrombectomy procedures are commonly performed at two vascular locations as shown in FIG. 21. Clots tend to settle at the M1 segment of the middle cerebral artery (MCA) and at the internal carotid artery (ICA). According to various embodiments of the present disclosure, target vasculature includes the M1 segment and the ICA. The M1 segment refers to the initial portion of the MCA, which extends from its origin at the ICA bifurcation to the point where it branches into the superior and inferior trunks. Occlusions at these areas are a frequent cause of large vessel strokes and are primary targets for endovascular intervention. The ICA target location refers to the intracranial portion of the internal carotid artery before it bifurcates into the MCA and anterior cerebral artery (ACA). Thrombectomy at the ICA is indicated when a clot is lodged proximally, often leading to more extensive cerebral ischemia. Timely intervention at either site can significantly improve neurological outcomes for patients with acute large vessel occlusions.

TABLE 1
Performance Update

| MCA (M1) | ICA |
|---|---|
| Vessel & Clot Dimensions | Vessel & Clot Dimensions |
| Vessel diameter range | Vessel diameter |
| is 2.5-3.5 mm | range is 4-6 mm |
| Bench Test | Double the volume of |
| 20% RBC | clot than in the M1 |
| 5 mm OD clot lodged | Bench Test |
| into 2.5 mm vessel | 20% RBC |
| 1.5 cm | 6 mm OD clot lodged |
| length | into 4.9 mm vessel |
| 5.5 sec average (n = 10) | 1.5 cm length |
| | 8 sec average (n = 5) |

In various control scenarios, aspiration alone was not sufficient to draw the clot into the aspiration catheter. A control test was performed before each test using exemplary systems described herein. The systems and methods of the present disclosure significantly improved clot disruption and aspiration having the characteristics described according to the results of Table 1 above.

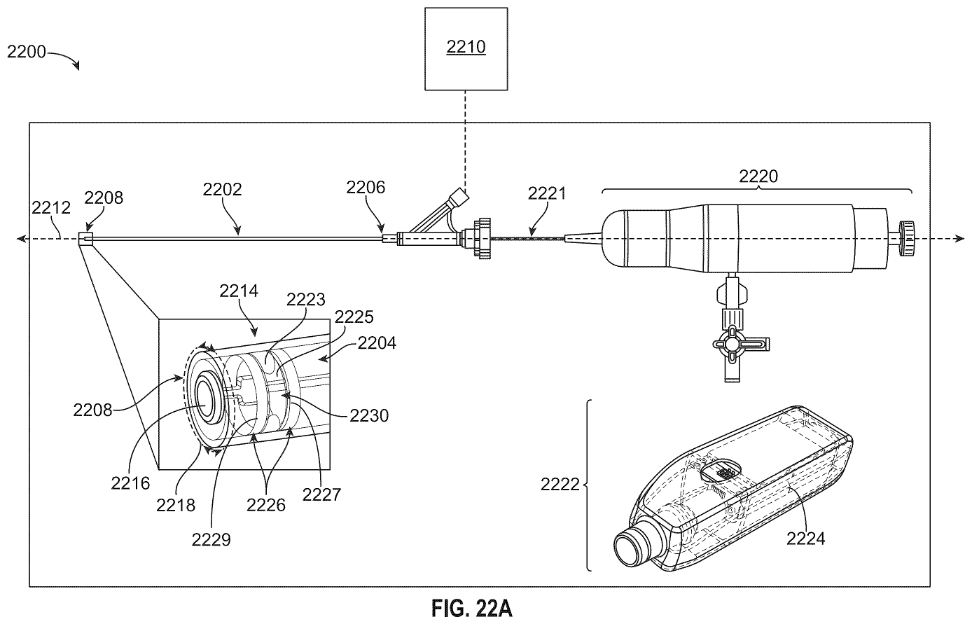

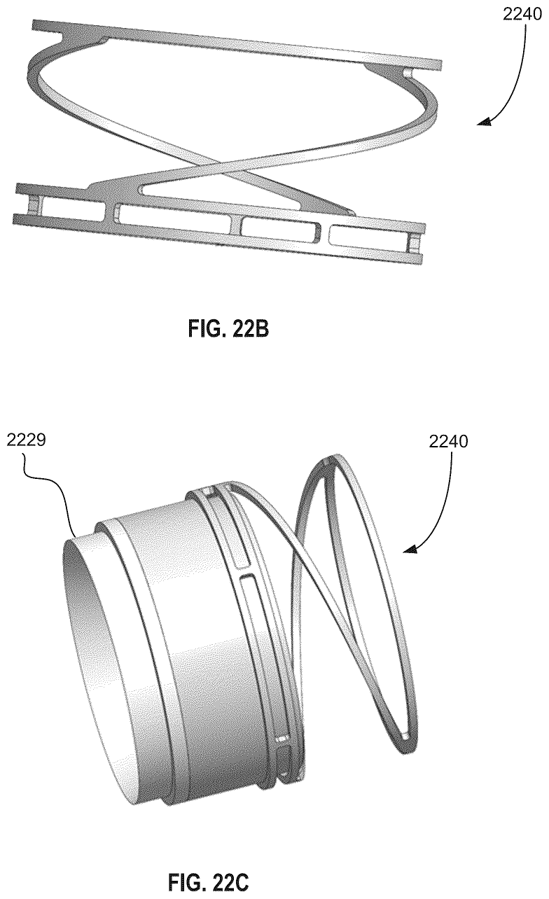

FIG. 22A illustrates an exemplary system for removing clot from a blood vessel, in accordance with various embodiments of the present disclosure. Clot removal system 2200 may include any combination of embodiments as previously described herein. System 2200 includes an aspiration catheter body 2202 having a clot-extraction lumen 2204 extending from a proximal end 2206 to an open distal region 2208. The proximal end 2206 is configured to be coupled to a vacuum source 2210. The open distal region 2208 includes a central axis 2212 extending therethrough.

System 2200 further includes a clot disruptor assembly 2214. According to various embodiments, the clot disruptor assembly 2214 may be preloaded into the aspiration catheter body 2202 prior to treatment and/or when the system 2200 is delivered to the health care professional. The clot disruptor assembly 2214 may include a substantially solid disc structure 2216 disposed transversely across at least a portion of the open distal region 2208. In at least some embodiments, the disc structure 2216 may be referred to as a disruptor. The disc structure 2216 may be configured to define a flow-blocked zone defined by a diameter of the disc structure 2216 and an open aspiration zone defined by the difference between the area between the diameter of the disc structure 2216 and a diameter of the lumen 2204 of the aspiration catheter body 2202. The disc structure 2216 may be substantially flush with the open distal region 2208 of the clot-extraction lumen 2204. Positioning the disc structure 2216 substantially flush with the opening provides the highest level of efficiency and safety. Positioning the disc structure 2216 too far inward lowers the efficiency of the clot removal and positioning the disc structure 2216 too far past the opening may become a patient safety concern.

The disc structure 2216 may include a planar structure oriented in a plane normal to the central axis 2212. In various embodiments, the disc structure 2216 is planar on at least a distal surface and has an average thickness in a range from 0.1 mm to 1 mm, inclusive. Furthermore, the disc structure 2216 has an average width or diameter in a range from 25% to 95%, inclusive, of an inner diameter of the open distal region 2208 of the clot-extraction lumen 2204.

The clot disruptor assembly 2214 may be further coupled to a driver 2221 that is configured to rotate and revolve the disc structure 2216 about the central axis 2212 to cause the open aspiration zone to travel in an orbital path 2218 about the central axis 2212 of the open distal region 2208. In some embodiments, the disc structure 2216 is asymmetrically positioned in the open distal region 2208 and is revolved about the central axis 2212 such that the aspiration zone is located between a peripheral edge of the disc structure 2216 and a rounded inner wall of the open distal region 2208 and follows an orbital path 2218 as the disc structure 2216 is revolved. For example, in at least some embodiments, the open distal region 2208 has a rounded inner wall and a cross-sectional area in a range from 1.2 mm² to 50 mm², inclusive, and the aspiration zone has a cross-sectional area in a range from 0.48 mm² to 40 mm², inclusive. Furthermore, the aspiration zone may have a cross-sectional area in a range from 40% to 80%, inclusive, of a cross-sectional area of the open distal region 2208.

In various embodiments, the clot disruptor assembly 2214 further includes a retention protrusion 2223 proximal to the disc structure 2216 and configured to movably engage with an annular guide assembly 2226. The annular guide assembly 2226 may extend from the lumen 2204 of the catheter body 2202. The retention protrusion 2223 may movably engage with the annular guide assembly 2226 to limit proximal and distal axial movement of the clot disruptor assembly 2214. The annular guide assembly 2226 may include two rings that extend from the lumen 2204 of the catheter body 2202. In various embodiments, the annular guide assembly 2226 includes a three-part construction stop band assembly including a middle spacer band 2225 having a first inner diameter and first outer diameter, and a proximal stop band 2227 and a distal stop band 2229 positioned on opposing axial sides of the middle spacer band 2225. The proximal stop band 2227 and distal stop band 2229 each have an inner diameter smaller than the inner diameter of the middle spacer band 2225 defining an annular guide channel 2230 therebetween when bonded to the middle spacer band 2229. The proximal stop band 2227 may be composed of stainless-steel alloys, cobalt chromium alloys, cobalt-based superalloys, or platinum iridium. The middle spacer band 2225 may be composed of stainless-steel alloys, cobalt chromium alloys, cobalt-based superalloys, or platinum iridium. The distal stop band 2229 may be composed of stainless-steel alloys, cobalt chromium alloys, cobalt-based superalloys, or platinum iridium. The bands are bonded together (e.g., by welding or adhesive bonding) in a concentrically aligned configuration sufficient to ensure that the annular ledges formed by the proximal stop band 2227 and distal stop band 2229 relative to the middle spacer band 2225 maintain substantially uniform radial height about the full circumference. In some embodiments, circumferential variation in ledge height is less than approximately 0.001 inches. Maintaining concentric alignment of the bonded bands is important to ensure reliable rotational retention of the clot disruptor assembly 2214 where the retention protrusion 2223 is disposed within the annular channel formed by the three-part construction stop band assembly. The inwardly projecting ledges formed by the proximal stop band 2227 and distal stop band 2229 define a radial capture height. If the rings are not concentrically aligned, circumferential variation in ledge height can occur, resulting in localized reductions in radial retention. At rotational speeds between 500 and 7000 RPM, even small eccentricities can generate dynamic radial forces and periodic displacement of the retention protrusion 2223. Excessive circumferential variation in the ledge geometry may therefore permit the retention protrusion 2223 to climb or migrate over a reduced-height region of the ledge, potentially allowing unintended escape from the annular guide channel 2230. Accordingly, concentricity between the bonded bands is controlled such that circumferential variation in ledge height is limited (e.g., less than approximately 0.001 inches), thereby maintaining consistent radial capture and preventing unintended radial displacement of the retention protrusion 2223 during high-speed operation.

According to various embodiments, the system 2200 further includes a handle 2220 coupled to or otherwise fixed to the driver 2221 and a power module 2222 including a motor 2224. The driver 221 may include a drive shaft (not shown) and the motor 2224 rotates the drive shaft about an axis parallel to the central axis 2212. For example, the motor 2224 may rotate the drive shaft about the central axis 2212. The power module 2222 may be detachably secured to the handle 2220 whereby the handle 2220 may be used to manipulate the driver 2221 in the clot-extraction lumen 2204 of the aspiration catheter body 2202. In some embodiments, the handle 2220 includes an input drive connector connected to the drive shaft and the power module 2222. The power module 2222 may include a battery, control circuitry, and an output drive connector connected to the motor 2224. The input and output drive connectors may include magnetic coupling elements for removably coupling the power module 2222 from the handle 2220 and vice versa.

In various embodiments, the disc structure 2216 may be configured to generate a clot flow through the open aspiration zone having an increased flow velocity and an increased dynamic pressure relative to a flow velocity and a dynamic pressure of a clot flow into the open distal region 2208 in the absence of the disc structure 2216. In at least some embodiments, applying a negative pressure to the proximal end 2206 of the lumen 2204 draws a flow of clot through the open aspiration zone and into the lumen 2204.

FIGS. 22B and 22C illustrate a stiffener ring 2240 that may be incorporated into the clot disruptor assembly 2214. Prolapse of the catheter tip may inadvertently expose the disc structure 2216 directly to a vessel wall which may, in turn, cause trauma to the vessel wall. The stiffener ring 2240 may be inserted into the lumen 2204 to mitigate prolapse of the open distal region 2208 of the catheter body 2202. The stiffener ring 2240 may be laser cut from a NiTi hypotube, according to at least some embodiments. Accordingly, the stiffener ring 2240 enables the open distal region 2208 of the catheter body 2202 to elastically collapse and return to its original shape after it is deformed due to the memory material of the stiffener ring 2240. In various embodiments, the stiffener ring 2240 may abut the distal stop band 2229 and be embedded within the wall of the lumen 2204. The stiffener ring 2240 may provide support for the open distal region 2208 of the catheter body 2202. In at least some embodiments, the stiffener ring 2240 may extend 0.254 mm past the distal stop band 2229.

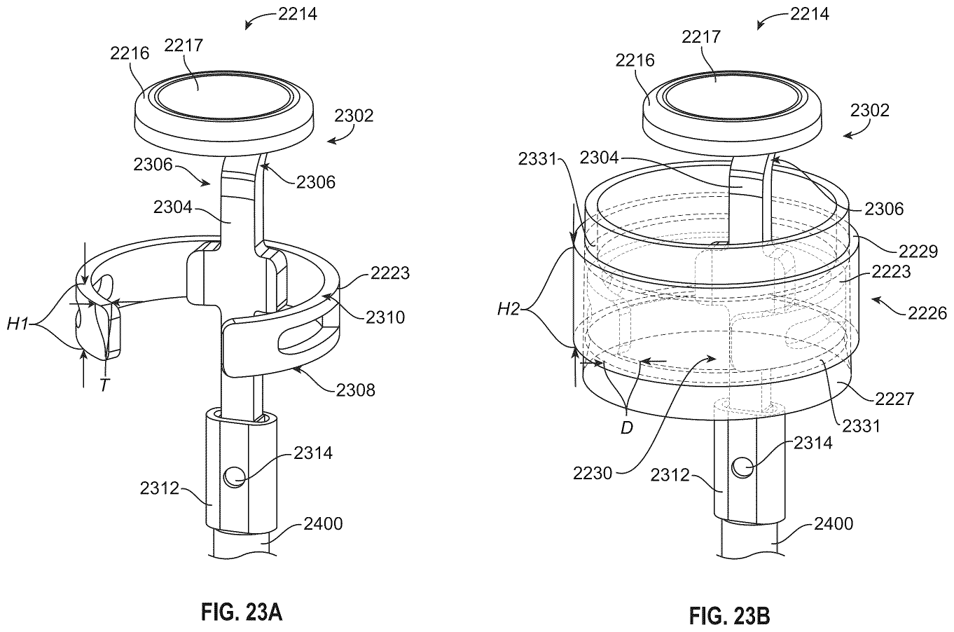

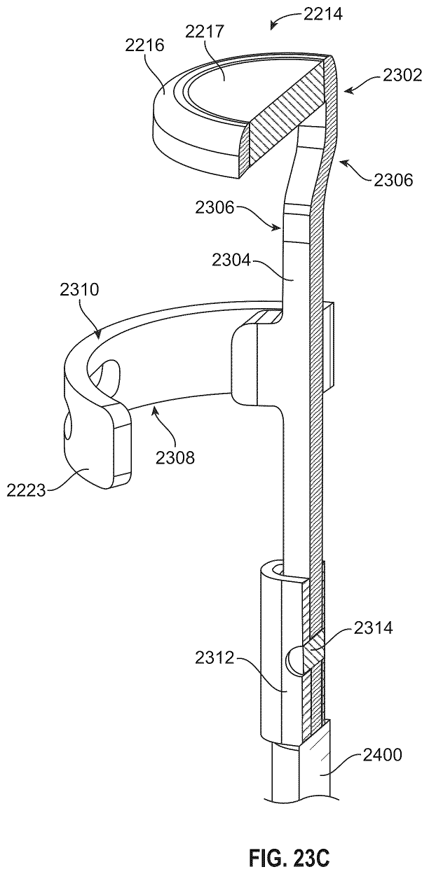

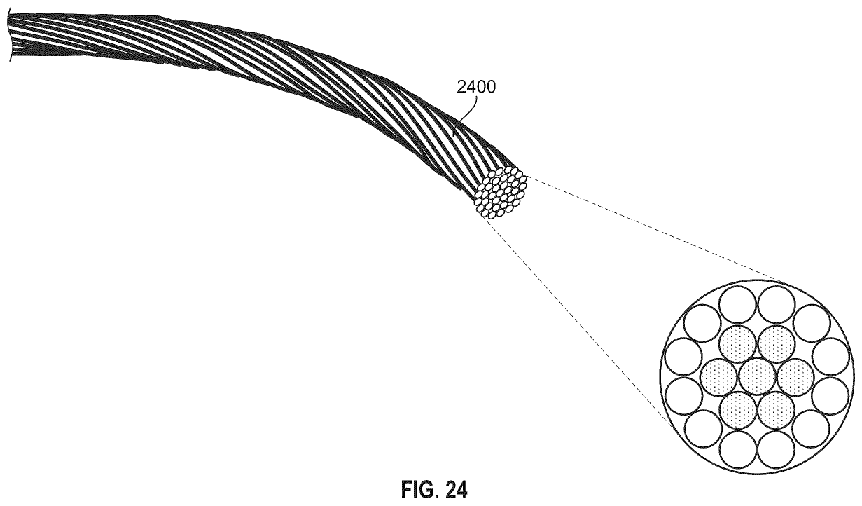

FIGS. 23A-23C illustrate an exemplary clot disruptor assembly, in accordance with various embodiments of the present disclosure. The clot disruptor assembly 2214 may include any combination of embodiments as described herein. In various embodiments, the clot disruptor assembly 2214 includes a disc structure 2216 as described in detail with respect to FIG. 22. The disc structure 2216 may be disposed at a distal end 2302 of the clot disruptor assembly 2214 and the disc structure 2216 is configured to partially occlude an opening into the lumen of an aspiration catheter. The disc structure 2216 provides an unexpected and surprising level of efficiency as it would be counterintuitive that a solid disc would efficiently break up clot material as the disc structure takes up a majority of the opening of the lumen 2204 and is blocked by the disc structure.

In some embodiments, at least a portion of the distal face of the disc structure 2216 is radiopaque and a portion of the annular guide assembly is radiopaque so as to facilitate engagement of the retention protrusion with the annular guide assembly. For example, the disc structure 2216 may include a filled material 2217 including tungsten, tantalum, gold, platinum, and stainless steel, barium sulfate, bismuth subcarbonate, bismuth trioxide, bismuth oxychloride, etc., or any combination thereof. In at least some embodiments, the disc structure 2216 includes a ring of NiTi and is solder filled with at least gold. A healthcare professional may monitor clot disruptor assembly 2214 and confirm that the disc structure 2216 having the filled material 2217 is advanced past the annular guide channel 2230 and the retention protrusion 2223 is clipped within the annular guide channel 2230.

In various embodiments, the clot disruptor assembly 2214 may further include a retention protrusion 2223 proximal to the disc structure 2216. For example, the retention protrusion 2223 is positioned between 1 mm and 5 cm from the disc structure 2216. The retention protrusion 2223 may include one or more of a retention ring (e.g., a full circular structure), one or more wing or arms, and a split ring. For example, the retention protrusion 2223 may include a circular retention ring. In various embodiments, the circular retention ring include a gap configured to allow the circular retention ring to elastically contract as the circular retention ring is inserted into the annular guide assembly 2226. In another example, the retention protrusion 2223 includes a C-shaped retention element having two wings separated by a gap as shown in FIGS. 23A-23B. According to various embodiments, the C-shaped configuration as shown and described herein advantageously reduces or mitigates any chatter caused by oscillation of the drive shaft. The retention protrusion 2223 may have an average thickness (T) in a range from 0.0762 mm to 0.1524 mm, inclusive. The retention protrusion 2223 may further have an axial height (H1) in a range from 0.127 mm to 1.0 mm, inclusive. In some embodiments, the axial height (H1) is about 0.381 mm.

The retention protrusion 2223 may be proximal to the disc structure 2216 and configured to movably engage with the annular guide assembly 2226. The annular guide assembly 2226 may extend from the lumen 2204 of the catheter body 2202. The retention protrusion 2223 may movably engage with the annular guide assembly 2226 to limit proximal and distal axial movement of the clot disruptor assembly 2214. The annular guide assembly 2226 may include two rings as shown in FIG. 22 that extend from the lumen 2204 of the catheter body 2202. In various embodiments, the annular guide assembly 2226 includes a three-part construction stop band assembly including a proximal stop band 2227 and a distal stop band 2229 defining an annular guide channel 2230 therebetween. The retention protrusion 2223 may be received within the annular guide channel 2230 of the annular guide assembly 2226. For example, the retention protrusion 2223 may be received between the proximal stop band 2227 and the distal stop band 2229.

In at least some embodiments, the disc structure 2216 has an outer diameter approximately equal to an inner diameter of the distal end region of the aspiration catheter lumen and includes a distal bearing surface 2308 which engages a distal bearing surface of the annular guide assembly 2226. The disc structure 2216 may further include a proximal bearing surface 2310 which engages a proximal bearing surface of the annular guide assembly 2226. The distal and proximal bearing surfaces on the annular guide assembly 2226 may be axially spaced apart to form an annular slot (e.g., the annular guide channel 2230) for rotationally receiving the disc structure 2216. The annular guide channel 2230 may have an axial height (H2) in range from 0.254 mm to 1.27 mm, inclusive. In various embodiments, an axial height (H2) is 0.508 mm. In some embodiments, an annular gap may exist between the retention protrusion 2223 and the annular guide channel 2230 when the retention protrusion 2223 is within the annular guide channel 2230 of the annular guide assembly 2226. The annular gap may be about 0.127 mm where H2-H1=0.508 mm-0.381 mm=0.127 mm. The annular gap may be in a range between 0.947 mm and 0.27 mm, in various embodiments. The annular guide channel 2230 may have an annular ledge 2231 having a depth (D) between 0.0254 mm to 0.2540 mm, inclusive. The annular guide channel 2230 may have an annular ledge 2231 at both a proximal and distal end of the annular guide channel 2230. The annular ledges 2231 may be symmetrical or asymmetrical. For example, the depth (D) may be different for each of a proximal annular ledge and a distal annual ledge.

In some embodiments, the clot disruptor assembly 2214 further includes an axial strut 2304. The axial strut 2304 may extend from or be part of a drive shaft 2400, to be described in further detail above. The retention protrusion 2223 and the disc structure 2216 may be joined by the axial strut 2304 as shown in FIGS. 23A-23C. For example, the retention protrusion 2223 and the disc structure 2216 may be laser welded to the axial strut 2304. The axial strut 2304 may include a connection point 2306 on a periphery of the disc structure 2216 and has a dog leg configured to position the connection point immediately adjacent to an inner wall of the distal end region go the lumen as the disc structure 2216 is revolved or rotated. A coupler tube 2312 and/or a coupler tube pin 2314 may be laser welded to couple the clot disruptor assembly 2214 to a drive shaft 2400, to be described in further detail below. In various embodiments, the drive shaft 2400 is stainless steel and the coupler tube 2312 is welded to the drive shaft 2400 and the axial strut 2304. The welded configuration mitigates any torsional force and resulting stress fractures.

FIG. 24 illustrates an exemplary elongated drive shaft, in accordance with various embodiments of the present disclosure. In various embodiments, the driver 2221 as referred to above includes a drive shaft 2400 coupled to the disc structure for rotating the disc structure. The drive shaft 2400 may include a two-component shaft. The drive shaft 2400 may be a multifilar torque strand formed in a helical pattern. The helical arrangement advantageously is able to at least partially untwist itself in response to becoming too stiff for moving within a tortuous vessel. For example, the individual filars in the drive shaft 2400 may be spaced apart from each other ever so slightly when the drive shaft 2400 is manufactured. The minute free space allows very small movements relative to each filar which enables the drive shaft 2400 to bend while still rotating with external torsional forces. The helical arrangement of the drive shaft 2400 increases the durability of the drive shaft 2400, reduces the friction inside the catheter, and protects against entanglement. The drive shaft 2400 may be coupled to a motor of a power module via a coupler, a crimp, welded, etc.

The drive shaft 2400 may include a distal end coupled to a disc structure at or near a peripheral edge thereof at a location diametrically opposed to the open aspiration zone. Rotation of the drive shaft 2400 may cause a distal length of the draft shaft to precess about the central axis. In various embodiments, the drive shaft 2400 is coupled to the disc structure and the drive shaft 2400 rotates the disc structure at around 4000 revolutions per minute. The drive shaft 2400 may eccentrically revolve the disc structure transversely around the opening of the lumen. The drive shaft 2400 may be offset within the catheter. The drive shaft 2400 may be rotated and revolved within the catheter, as described in detail with respect to the foregoing figures. The combination of the rotating and revolving drive shaft 2400 with the clot disruptor assembly may act as a “potato slicer” with respect to the clot in that it may “slice” the clot as part of the disruption and aspiration from the vasculature.

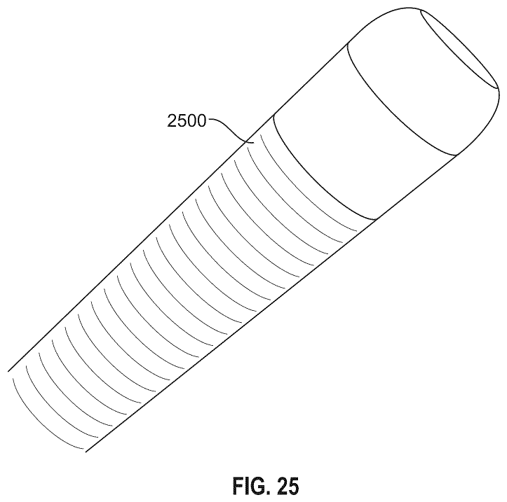

FIG. 25 illustrates an exemplary a non-rotating sleeve, in accordance with various embodiments of the present disclosure. The non-rotating sleeve 2500 may be used in combination with any of the embodiments as described herein. The non-rotating sleeve 2500, also referred to herein as an outer sleeve, may include a longitudinal passage therethrough. In various embodiments, the drive shaft 2400 as shown and described with respect to FIG. 24 is rotated within a non-rotating sleeve 2500 extending through the lumen of an aspiration catheter (e.g., aspiration catheter body 2202) such that the drive shaft 2400 and the non-rotating sleeve 2500 are coaxial. The non-rotating sleeve 2500 advantageously prevents or mitigates entanglement of clot material during rotation. The non-rotating sleeve 2500 further adds stiffness to the system. At least a portion of the drive shaft 2400 and/or the non-rotating sleeve 2500 is covered with a lubricious cover or coating (not shown) for reducing friction during rotation and revolution. In various embodiments, the non-rotating sleeve 2500 is configured to mitigate any air from entering between the drive shaft 2400 and the non-rotating sleeve 2500. The non-rotating sleeve 2500 further reduces chatter within the catheter and reduces the pressure load on the clot disruptor assembly. The non-rotating sleeve 2500 may extend over at least a proximal length of the drive shaft 2400 and is non-rotatably disposed over the drive shaft 2400. In various embodiments, the non-rotating sleeve 2500 terminates about 3 cm from the disc structure for increased flexibility of the drive shaft 2400.

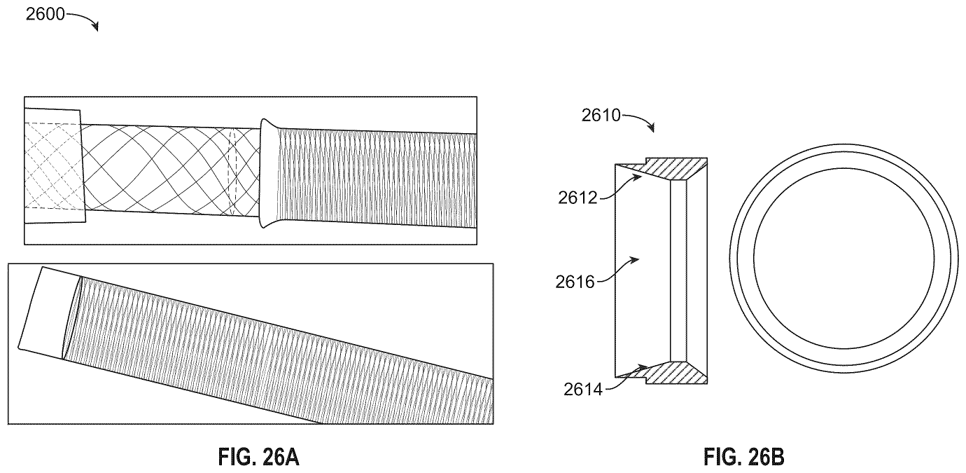

FIGS. 26A-26B illustrate an exemplary aspiration catheter, in accordance with embodiments of the present disclosure. The aspiration catheter 2600 may include a three-part construction stop band assembly 2610 integrated with the aspiration catheter as shown in the cross-section of FIG. 26B including a proximal stop band 2612 and a distal stop band 2614 defining an annular guide channel 2616 therebetween. According to some embodiments, a retention protrusion of a clot disruptor assembly may be disposed between the proximal stop band 2612 and the distal stop band 2614. One or more of the proximal stop band 2612 and the distal stop band 2614 may have an annular guide clearance of about 0.5 mm. The three-part construction stop band assembly 2610 may be a particular embodiment of the annular guide assembly as described throughout the present disclosure.

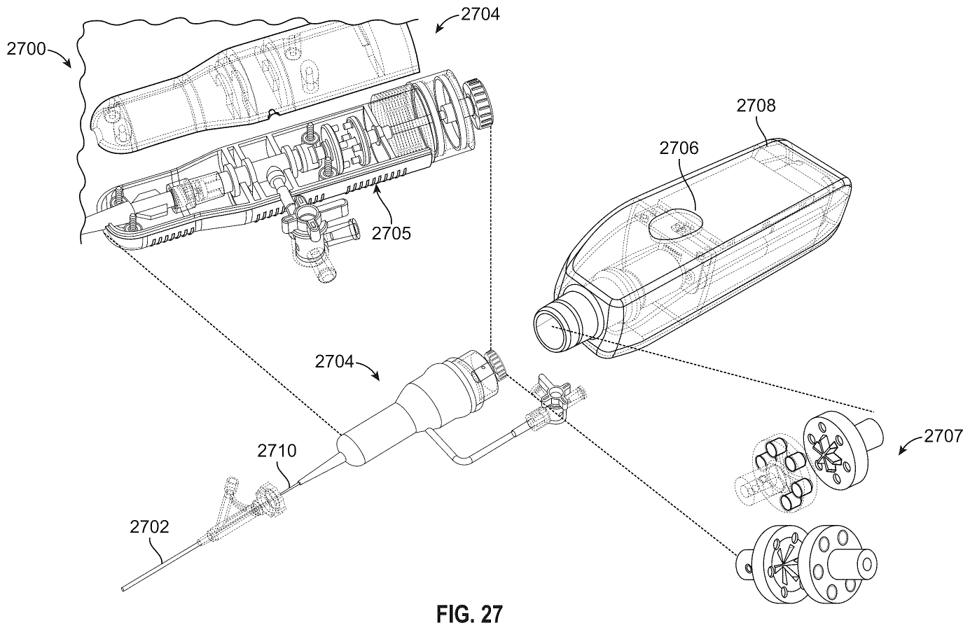

FIG. 27 illustrates an exemplary system, in accordance with embodiments of the present disclosure. System 2700 may include any of the embodiments as described in detail herein. The system may be about 0.071 in ID and have a 155 cm effective length. The system 2700 may be compatible with a 0.088 in guide catheter, according to various embodiments. For example, the system 2700 may be compatible with a Microcath and a Tenzing 5. Furthermore, the system 2700 may be compatible with a 0.035″ guide wire. The aspiration catheter 2702 may have a 6.5 F profile and a 0.071″ ID with a 145 cm effective length, in at least some embodiments. The handle 2704 may include a hybrid draft shaft construction.

In various embodiments, the handle 2704 may include a clacker assembly 2705 that advantageously provides an audible “clacker” for indicating the motor 2706 of the power module 2708 in “on.” For example, the clacker assembly 2705 may include extensions that create noise as an audio alert for the health care professional. The handle 2704 may have a 155 cm effective length. The handle 2704 may be magnetically coupled via corresponding magnetic couplers 2707 to the power module 2708. In various embodiments, the magnetic couplers 2707 are configured to remain coupled and maintain a constant torque response at a distal end of the drive shaft 2710. In some embodiments, the motor 2706 is a brushless motor. The power module 2708 may further include a printed control board (PCB) for rotating and revolving the drive shaft 2710 according to modifiable parameters.

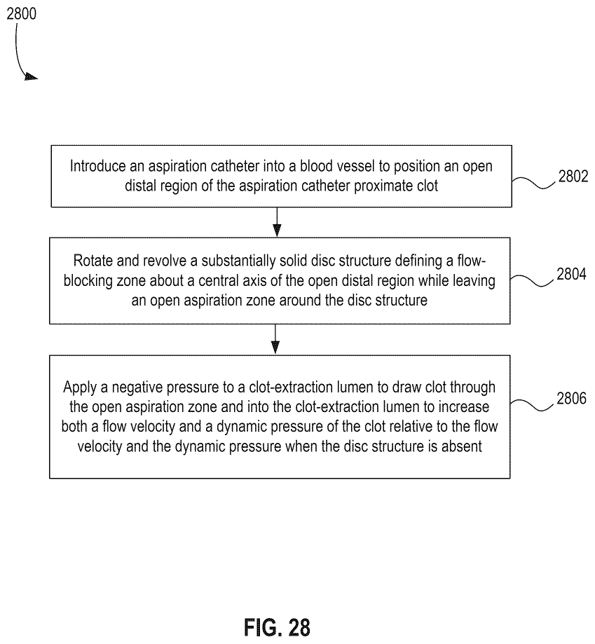

FIG. 28 is a flowchart of a method for removing clot from a blood vessel. Method 2800 may include more or less operations than those explicitly disclosed herein. The operations may be performed in alternative orders than those explicitly described herein unless otherwise specifically stated. Method 2800 may employ any of the embodiments described within the present disclosure including any devices or systems for removing clot as laid out above. Method 2800 may include operation 2802. Operation 2802 includes introducing an aspiration catheter into a blood vessel to position an open distal region of the aspiration catheter proximate clot. In various embodiments, the aspiration catheter and flow-blocking disc structure are positioned in a middle cerebral artery for the treatment of ischemic stroke. In other embodiments, the aspiration catheter and flow-blocking disc structure are positioned in an internal carotid artery for the treatment of ischemic stroke. The catheter may be advanced against the clot material to aspirate the clot material into the lumen of the catheter while rotating the drive shaft to dynamically compress the clot material between an edge of the revolving disruptor and an inner diameter of the opening. Advancing the catheter against the clot material may alter the viscosity of the clot material for aspirating the clot material into the lumen of the catheter.

In at least some embodiments, method 2800 includes advancing a clot disruptor assembly, including the flow-blocking disc, within a lumen of the aspiration catheter until a retention protrusion of the disruptor assembly engages an annular guide assembly that is between 1 mm and 5 cm of a distal end of the lumen so that a disruptor at a distal end of the disruptor assembly is positioned eccentrically across an opening into the lumen of the catheter to occlude a portion of the opening. The clot disruptor assembly and the annular guide assembly may include any of the embodiments described in detail above. The retention protrusion may be retained within the annular guide assembly to limit proximal and distal axial movement of the disruptor assembly in the lumen.

In some embodiments, engaging the retention protrusion within the annular guide assembly includes engaging the retention protrusion against or between one or more of a ring forming the annular guide assembly, a pair of rings forming the annular guide assembly, and a channel forming the annular guide assembly. In at least some embodiments, engaging the retention protrusion within the annular guide assembly includes engaging a C-shaped retention protrusion having two wings that engage a proximal stop band and a distal stop band of the annular guide assembly.

Operation 2804 may include rotating and revolving a substantially solid disc structure defining a flow-blocking zone about a central axis of the open distal region while leaving an open aspiration zone around the disc structure. In various embodiments, operation 2804 may include rotating and revolving the disc structure to revolve the open aspiration zone about an axis of the open distal region. The flow-blocking disc structure may be positioned transversely across the open distal region. The disc structure may be coupled to a drive shaft and operation 2804 may include rotating the drive shaft around 4000 revolutions per minute. The drive shaft may change direction of rotation throughout the procedure. In at least some embodiments, method 2800 includes reversing a direction of the revolutions every 0.5 seconds with a 0.1 millisecond pause therebetween. The healthcare professional may successfully and efficiently proceed with a program including the foregoing combination of 4000 revolutions per minute and reversing a direction of the revolutions every 0.5 seconds with a 0.1 second pause.

In various embodiments, the flow-blocking disc structure may be positioned across the open distal region before introducing the aspiration catheter into the blood vessel. The disruptor assembly including the flow-blocking disc may be advanced within the lumen of the aspiration catheter before positioning the aspiration catheter into the blood vessel. For example, the flow-blocking disc structure may be pre-assembled prior to blood vessel introduction. This pre-assembly is advantageous for efficient clinical use, and it facilitates coupling and locking of the retention ring within the annular guide assembly prior to patient introduction.

Operation 2806 may include applying a negative pressure to a clot-extraction lumen to draw clot through the open aspiration zone and into the clot-extraction lumen to increase both a flow velocity and a dynamic pressure of the clot relative to the flow velocity and the dynamic pressure when the disc structure is absent. In various embodiments, the flow velocity is increased by a factor of at least 1.5 and the dynamic pressure is increased by a factor of at least two. The negative pressure may be applied to the clot-extraction lumen for a period of time in a range from 5 seconds to 30 seconds, inclusive, for substantial removal of the clot from the middle cerebral artery in a single pass treatment. The negative pressure may be applied to the clot-extraction lumen for a period of time in a range from 5 seconds to 60 seconds, inclusive, for substantial removal of the clot from the internal carotid artery in a single pass treatment. These ranges are significantly reduced compared to convention systems where similar clot removal procedures are on the order several minutes. In various embodiments, a procedure using the systems and methods described herein may be about 2 minutes.

Reference Nos.

| | |
|---|---|
| 100 | Clot extraction catheter |
| 102 | Catheter body |
| 104 | Open distal region |
| 106 | Clot-disruption structure (e.g., disruptor assembly) |
| 108 | Retention ring |
| 110 | Clot-disruption element (e.g., disc-like disruptor) |
| 110a | Distal facing surface |
| 112 | Connecting strut |
| 114 | Drive cable |
| 114a | Distal end of drive cable |
| 116 | Proximal port |
| 120 | Proximal hub |
| 122 | Drive motor |
| 124 | Vacuum port |
| 126 | Clot-extraction lumen |
| 128 | Outer frame |
| 130 | Distal stop band |
| 132 | Proximal bearing surface |
| 138 | Distal bearing surface |
| 142 | Strut connection point |
| 144 | Directional vector |
| 150 | Central axis |
| 160 | Annular path |
| 162 | Broken lines |
| 164 | RO filler |
| 166 | Outer region |
| 168 | Inner wall |
| 170 | Drive cable assembly |
| 172 | Outer sleeve |
| 172a | Distal end of outer sleeve |
| 174 | Rotational arrow |
| 176 | Revolution arrow |
| 180 | Clot extraction catheter |
| 182 | Catheter body |
| 184 | Open distal region |
| 186 | Clot-disruption structure (e.g., disruptor assembly) |
| 188 | Retention ring |
| 190 | Ring-like clot-disruption element (disruptor) |
| 192 | Connecting strut |
| 194 | Drive cable |
| 198 | Distal stop band |
| 200 | Clot extraction catheter |
| 202 | Catheter body |
| 204 | Open distal end |
| 206 | Clot-disruption structure (e.g., disruptor assembly) |
| 208 | Retention ring |
| 210 | Clot-disruption element (e.g., disruptor) |
| 211 | Outer frame |
| 212 | RO filler |
| 214 | Drive cable |
| 216 | Distal stop band |
| 300 | Clot-disruption structure (e.g., disruptor assembly) |
| 302 | C-shaped retention ring |
| 304 | Clot-disruption element (e.g., disruptor) |
| 306 | Connecting strut |
| 307 | Wing |
| 308 | Gap |
| 309 | Bridging section |
| 310 | Cutout |
| 312 | Dog leg |
| 313 | Connection point |
| 314 | Line |
| 320 | Open distal end |
| 322 | Distal stop band |
| 324 | Proximal stop band |
| 330 | Drive cable |
| 332 | Outer sleeve |
| 340 | Stop band assembly |
| 342 | Distal stop band |
| 344 | Proximal stop band |
| 346 | Annular guide channel |
| 360 | Handle |
| 362 | Housing |
| 364 | Motor |
| 366 | Flush port |
| 380 | Handle Assembly |
| 382 | Distal housing |
| 384 | Proximal housing |
| 386 | Flush line |
| 387 | Valve |
| 388 | Motor |
| 390 | Battery |
| 394 | Proximal drive connector |
| 396 | Distal drive connector |
| 397 | Drive shaft |
| 398 | Magnets |
| 400 | Mechanical interlock |
| 402 | Tubular manifold |
| 404 | Drive shaft |
| 406 | Clackers |
| 406a | Clacker |
| 406b | Clacker |
| 408 | Control circuitry |
| 410 | External control |
| 412 | Internal switches |
| 500 | Clot-disruption structure (e.g., disruptor assembly) |
| 502 | C-shaped retention ring |
| 504 | Clot-disruption element (e.g., disruptor) |
| 506 | Connecting strut |
| 508 | Wing |
| 510 | Gap |
| 512 | Bridging section |
| 514 | Notches |
| 516 | Retention tabs |
| 518 | Coupling sleeve |
| 520 | Fastening pin |
| 540 | Annular Guide Assembly |
| 542 | Proximal ring |
| 544 | Ramp |
| 546 | Annular groove |
| 548 | Distal ring |
| 550 | Distal stop surface |
| 552 | Proximal stop surface |
| 580 | Flow bocking disc |
| 580a | Flow bocking disc |
| 580b | Flow bocking disc |
| 580c | Flow bocking disc |
| 580d | Flow bocking disc |
| 582 | Open passage |
| 2200 | Clot removal system |
| 2202 | Aspiration catheter body |
| 2204 | Aspiration catheter lumen |
| 2206 | Proximal end |
| 2208 | Open distal region |
| 2210 | Vacuum source |
| 2212 | Axis |
| 2214 | Clot disruptor assembly |
| 2216 | Disc structure |
| 2217 | Filled material |
| 2218 | Orbital path |
| 2220 | Handle |
| 2221 | Driver |
| 2222 | Power module |
| 2224 | Motor |
| 2225 | Middle stop band |
| 2226 | Annular guide assembly |
| 2227 | Proximal stop band |
| 2229 | Distal stop band |
| 2230 | Annular guide channel |
| 2231 | Annular ledge |
| 2240 | Stiffener ring |
| 2302 | Clot disruptor assembly distal end |
| 2304 | Axial strut |
| 2306 | Connection point |
| 2308 | Distal bearing surface |
| 2310 | Proximal bearing surface |
| 2312 | Coupler tube |
| 2314 | Coupler tube pin |
| 2400 | Drive shaft |
| 2500 | Non-rotation sleeve |
| 2600 | Aspiration catheter |
| 2610 | Three-part stop band construction assembly |
| 2612 | Proximal stop band |
| 2614 | Distal stop band |
| 2616 | Annular guide channel |
| 2700 | System |
| 2702 | Aspiration catheter |
| 2704 | Handle |
| 2705 | Clacker assembly |
| 2706 | Motor |
| 2707 | Magnetic couplers |
| 2708 | Power module |
| 2710 | Drive shaft |

All publications and patent applications mentioned in this specification are herein incorporated by reference in their entirety to the same extent as if each individual publication or patent application was specifically and individually indicated to be incorporated by reference.

### Examples

Example 1: A system for removing clot from a blood vessel, the system comprising: an aspiration catheter body having a clot-extraction lumen extending from a proximal end to an open distal region, wherein the proximal end is configured to be coupled to a vacuum source and the open distal region has a central axis therethrough; a substantially solid disc structure disposed transversely across at least a portion of the open distal region, said disc structure configured to define a flow-blocked zone and an open aspiration zone through the open distal region; and a driver configured to rotate and revolve the disc structure about the central axis to cause the open aspiration zone to travel in an orbital path about the central axis of the open distal region; wherein applying a negative pressure to the proximal end of the clot-extraction lumen draws a flow of clot through the open aspiration zone and into the clot-extraction lumen.

Example 2: A system according to Example 1, wherein the disc structure is configured to generate a clot flow through the open aspiration zone having an increased flow velocity and an increased dynamic pressure relative to a flow velocity and a dynamic pressure of a clot flow into the open distal region in the absence of the disc structure.

Example 3: A system according to any of Examples 1-2, wherein the disc structure is asymmetrically positioned in the open distal region and is revolved about the central axis and wherein the aspiration zone is located between a peripheral edge of the disc structure and a rounded inner wall of the open distal region and follows an orbital path as the disc structure is revolved.

Example 4: A system according to any of Examples 1-3, wherein the driver comprises a drive shaft having a distal end coupled to the disc structure at or near a peripheral edge thereof at a location diametrically opposed to the open aspiration zone, wherein rotation of the drive shaft causes a distal length of the drive shaft to precess about the central axis.

Example 5: A system according to any of Examples 1-4, wherein the disc structure comprises a planar structure oriented in a plane normal to the central axis.

Example 6: A system according to any of Examples 1-5, wherein the open distal region has a rounded inner wall and a cross-sectional area in a range from 1.2 mm² to 50 mm² and the aspiration zone has a cross-sectional area in a range from 0.48 mm² to 40 mm².

Example 7: A system according to any of Examples 1-6, wherein the aspiration zone has a cross-sectional area in a range from 40% to 80% of a cross-sectional area of the open distal region.

Example 8: A system according to any of Examples 1-7, wherein the disc structure is planar on at least a distal surface and has an average thickness in a range from 0.1 mm to 1 mm.

Example 9: A system according to Example 8, wherein the disc structure has an average width or diameter in a range from 25% to 95% of an inner diameter of the open distal region of the clot-extraction lumen.

Example 10: A system according to Example 8, wherein the disc structure is substantially flush with the open distal region of the clot-extraction lumen.

Example 11: A system according to any of Examples 1-10, wherein the driver comprises a drive shaft and a motor configured to rotate the drive shaft about an axis parallel to the central axis.

Example 12: A system according to Example 11, wherein the driver further comprises an outer sleeve having a longitudinal passage therethrough, wherein the outer sleeve is non-rotatably disposed over the drive shaft.

Example 13: A system according to Example 12, wherein at least a portion of the drive shaft or sleeve is covered with a lubricous cover or coating.

Example 14: A system according to any of Examples 12-13, wherein the driver comprises (1) a handle fixed to the drive shaft and the outer sleeve and (2) a module including the motor detachably secured to the handle, whereby the handle may be used to manipulate the drive shaft and sleeve in the clot-extraction lumen of the aspiration catheter prior to attaching the power module.

Example 15: A system according to Example 14, wherein the handle comprises an input drive connector connected to the drive shaft and the power module includes a battery, control circuitry, and an output drive connector connected to the motor, wherein the input and output drive connectors comprise magnetic coupling elements.

Example 16: A system for removing clot from a blood vessel, comprising: an aspiration catheter having a lumen extending therethrough, an opening into the lumen, and an annular guide assembly within a distal end region of the lumen; and a clot disruptor assembly configured to be positioned within the catheter, the disruptor assembly comprising: a disruptor at a distal end of the disruptor assembly having a distal face that is configured to partially occlude the opening into the lumen of the catheter; a retention protrusion proximal to the disruptor and configured to movably engage the annular guide assembly of the catheter to limit proximal and distal axial movement of the disruptor assembly in the lumen; and an elongated drive shaft eccentrically coupled to the disruptor and configured to be rotated to drive revolution of the disruptor across the opening into the lumen of the catheter when the retention protrusion is engaged with the annular guide assembly of the catheter.

Example 17: A system according to Example 16, wherein the annular guide assembly comprises a three-part construction stop band assembly including a proximal stop band and a distal stop band defining an annular guide channel therebetween.

Example 18: A system according to Example 17, wherein the annular guide channel has an axial height in range from 0.254 mm to 1.27 mm.

Example 19: A system according to any of Examples 16-18, wherein the annular guide assembly is between 2 mm and 5 cm from a distal end of the catheter.

Example 20: A system according to any of Examples 16-19, wherein the retention protrusion has an axial height in a range from 0.127 mm to 1.0 mm.

Example 21: A system according to any of Examples 16-20, wherein the distal face of the disruptor is configured to occlude between 30%-95% of the opening into the lumen of the catheter when the retention protrusion is engaged with the annular guide assembly.

Example 22: A system according to any of Examples 16-21, wherein the disruptor comprises a disc-shaped disruptor having a planar structure oriented in a plane normal to a central axis of the catheter lumen.

Example 23: A system according to any of Examples 16-22, wherein at least a portion of the distal face of the disruptor is radiopaque and a portion of the annular guide assembly is radiopaque so as to facilitate engagement of the retention protrusion with the annular guide assembly.

Example 24: A system according to any of Examples 16-23, wherein the retention protrusion is positioned between 1 mm and 5 cm from the disruptor.

Example 25: A system according to any of Examples 16-24, wherein the disruptor is substantially flush with the lumen opening.

Example 26: A system according to any of Examples 16-25, wherein the retention protrusion comprises one or more of: a retention ring, one or more wings or arms, and a split ring.

Example 27: A system according to any of Examples 16-26, wherein the retention protrusion comprises a circular retention ring configured to be removably received in an annular guide channel of the annular guide assembly disposed on an inner wall of the distal end region of the lumen.

Example 28: A system according to Example 27, wherein the circular retention ring is disposed between distal and proximal stop bands of the annular guide assembly.

Example 29: A system according to any of Examples 27-28, wherein the circular retention ring has an outer diameter approximately equal to an inner diameter of the distal end region of the aspiration catheter lumen and comprises a distal bearing surface which engages a distal bearing surface of the annular guide assembly.

Example 30: A system according to Example 29, wherein the circular retention ring further comprises a proximal bearing surface which engages a proximal bearing surface of the annular guide assembly.

Example 31: A system according to Example 30, wherein the distal and proximal bearing surfaces on the annular guide assembly are axially spaced apart to form an annular slot for rotationally receiving the circular retention ring.

Example 32: A system according to any of Examples 27-31, wherein the circular retention ring comprises a gap configured to allow the circular retention ring to elastically contract as the circular retention ring is inserted into the annular guide assembly.

Example 33: A system according to any of Examples 27-32, wherein the disruptor and the circular retention ring are joined by an axial strut.

Example 34: A system according to Example 33, wherein a distal end of the axial strut is connected to a connection point on a periphery of the circular retention ring and has a dog leg configured to position the connection point immediately adjacent to an inner wall of the distal end region of the lumen as the disruptor is revolved or rotated.

Example 35: A system according to any of Examples 27-34, wherein the circular retention ring comprises a C-shaped retention element having two wings separated by a gap.

Example 36: A system according to any of Examples 27-35, further comprising a non-rotating sleeve extending over at least a proximal length of the drive shaft, wherein the drive shaft is configured to rotate relative to the sleeve.

Example 37: A method for removing clot from a blood vessel, the method comprising: introducing an aspiration catheter into a blood vessel to position an open distal region of the aspiration catheter proximate clot; rotating and revolving a substantially solid disc structure defining a flow-blocking zone about a central axis of the open distal region while leaving an open aspiration zone around the disc structure; and applying a negative pressure to a clot-extraction lumen to draw clot through the open aspiration zone and into the clot-extraction lumen to increase both a flow velocity and a dynamic pressure of the clot relative to the flow velocity and the dynamic pressure when the disc structure is absent.

Example 38: A method according to Example 37, further comprising positioning the flow-blocking disc structure transversely across the open distal region.

Example 39: A method according to Example 38, wherein the flow-blocking disc structure is positioned across the open distal region before introducing the aspiration catheter into the blood vessel.

Example 40: A method according to any of Examples 37-39, wherein the flow velocity is increased by a factor of at least 1.5 and the dynamic pressure is increased by a factor of at least two.

Example 41: A method according to any of Examples 37-40, further comprising rotating and revolving the disc structure to revolve the open aspiration zone about an axis of the open distal region.

Example 42: A method according to any of Examples 37-41, wherein the open distal region has a cross-sectional area in a range from 1.2 mm² to 50 mm² and the aspiration zone has a cross-sectional area in a range from 0.48 mm² to 40 mm².

Example 43: A method according to any of Examples 37-42, wherein the aspiration zone has a cross-sectional area in a range from 40% to 80% of a cross-sectional area of the distal region.

Example 44: A method according to any of Examples 37-43, wherein the disc structure is planar on at least a distal surface and has an average thickness in a range from 0.1 mm to 1 mm.

Example 45: A method according to any of Examples 37-44, wherein the disc structure has a width which is less than an inner diameter of the open distal region and the aspiration zone is located between an outer periphery of the disc structure and an inner wall of the open distal region.

Example 46: A method according to any of Examples 37-45, wherein the aspiration catheter and flow-blocking disc structure are positioned in a middle cerebral artery for the treatment of ischemic stroke.

Example 47: A method according to Example 46, wherein the negative pressure is applied to the clot-extraction lumen for a period of time in a range from 5 seconds to 30 seconds for substantial removal of the clot from the middle cerebral artery in a single pass treatment.

Example 48: A method according to any of Examples 37-45, wherein the aspiration catheter and flow-blocking disc structure are positioned in an internal carotid artery for the treatment of ischemic stroke.

Example 49: A method according to Example 48, wherein the negative pressure is applied to the clot-extraction lumen for a period of time in a range from 5 seconds to 60 seconds for substantial removal of the clot from the internal carotid artery in a single pass treatment.

Example 50: A method according to any of Examples 37-49, wherein the disc structure is coupled to a drive shaft, further comprising rotating the drive shaft around 4000 revolutions per minute.

Example 51: A method according to Example 50, further comprising reversing a direction of the revolutions every 0.5 seconds with a 0.1 millisecond pause therebetween.

Example 52: A method for removing a clot material from a blood vessel, the method comprising: advancing a disruptor assembly within a lumen of an aspiration catheter until a retention protrusion of the disruptor assembly engages an annular guide assembly that is between 1 mm and 5 cm of a distal end of the lumen so that a disruptor at a distal end of the disruptor assembly is positioned eccentrically across an opening into the lumen of the catheter to occlude a portion of the opening; rotating a drive shaft to eccentrically revolve the disruptor transversely around the opening while retaining the retention protrusion within the annular guide assembly to limit proximal and distal axial movement of the disruptor assembly in the lumen; and applying aspiration through the lumen.

Example 53: A method according to Example 52, further comprising advancing the catheter against a clot material to aspirate the clot material into the lumen of the catheter while rotating the drive shaft to dynamically compress the clot material between an edge of the revolving disruptor and an inner diameter of the opening.

Example 54: A method according to Example 52 or 53, further comprising advancing the catheter against a clot material to alter the viscosity of the clot material and aspirate the clot material into the lumen of the catheter.

Example 55: A method according to any of Examples 52-54, wherein rotating the drive shaft comprises rotating the drive shaft within a non-rotating sleeve extending through the lumen of the catheter.

Example 56: A method according to any of Examples 52-55, wherein engaging the retention protrusion within the annular guide assembly comprises engaging one or more of: a retention ring forming the retention protrusion, a split ring forming the retention protrusion, one or more wings forming the retention protrusion.

Example 57: A method according to any of Examples 52-56, wherein engaging the retention protrusion within the annular guide assembly comprises engaging the retention protrusion against or between one or more of: a ring forming the annular guide assembly, a pair of rings forming the annular guide assembly, and a channel forming the annular guide assembly.

Example 58: A method according to any of Examples 52-55, wherein engaging the retention protrusion within the annular guide assembly comprises engaging a C-shaped retention protrusion having two wings that engage a proximal stop band and a distal stop band of the annular guide assembly.

Example 59: A method according to any of Examples 52-58, wherein the disruptor assembly is positioned eccentrically across the opening into the lumen of the catheter to occlude between 30-95% of the opening.

Example 60: A method according to any of Examples 52-59, further comprising positioning the aspiration catheter within a blood vessel, wherein the advancing the disruptor assembly within the lumen of the aspiration catheter is carried out before positioning the aspiration catheter into the blood vessel.

Example 61: A method for removing clot from a blood vessel, the method comprising: introducing an aspiration catheter into a blood vessel to position an open distal region of the aspiration catheter proximate clot; revolving and/or rotating a flow-blocking plate structure about a central axis of the open distal region while leaving an open aspiration zone around or through the plate structure; and applying a negative pressure to a clot-extraction lumen to draw clot through the aspiration zone and into the clot-extraction lumen to increase both a flow velocity and a dynamic pressure of the clot relative to the flow velocity and the dynamic pressure when the plate structure is absent.

Example 62: A method according to Example 61, further comprising positioning the flow-blocking plate structure transversely across the open distal region.

Example 63: A method according to Example 62, wherein the flow-blocking plate structure is positioned across the open distal region before introducing the aspiration catheter into the blood vessel.

Example 64: A method according to Example 62, wherein the flow-blocking plate structure is positioned across the open distal region after introducing the aspiration catheter into the blood vessel.

Example 65: A method according to Example 64, wherein the flow-blocking plate structure is positioned in the clot extraction lumen while the aspiration catheter is being introduced into the blood vessel and positioned across the open distal region after the open distal region is proximate the clot.

Example 66: A method according to any of Examples 61-65, wherein the flow velocity is increased by a factor of at least 1.5 and the dynamic pressure is increased by a factor of at least two.

Example 67: A method according to any of Examples 61-66, further comprising revolving or rotating the plate structure to revolve the open aspiration zone about an axis of the open distal region.

Example 68: A method according to any of Examples 61-67, wherein the open distal region has a cross-sectional area in a range from 1.2 mm² to 50 mm².

Example 69: A method according to Example 68, wherein the aspiration zone has a cross-sectional area in a range from 0.48 mm² to 40 mm².

Example 70: A method according to any of Examples 61-67, wherein a cross-sectional area of the aspiration zone has a cross-sectional area in a range from 40% to 80% of a cross-sectional area of the distal region.

Example 71: A method according to any of Examples 61-70, wherein the plate structure comprises a disc.

Example 72: A method according to Example 71, wherein the disc has a thickness in a range from 0.076 mm to 0.305 mm.

Example 73: A method according to Example 71 or 72, wherein the disc is planar on at least a distal surface.

Example 74: A method according to any of Examples 71-73, wherein the disc has a rounded periphery.

Example 75: A method according to Example 74, wherein the disc has a circular periphery.

Example 76: A method according to any of Examples 71-75, wherein the disc has a width which is less than an inner diameter of the open distal region and the aspiration zone is located between an outer periphery of the disc and an inner wall of the open distal region.

Example 77: A method according to Example 76, wherein the disc is revolved about the axis of the open distal region to revolve within the aspiration zone.

Example 78: A method according to any of Examples 71-75, wherein the disc has a width which is substantially equal to an inner diameter of the open distal region.

Example 79: A method according to Example 78, wherein the aspiration zone is located asymmetrically through the disc.

Example 80: A method according to Example 79, wherein the disc is rotated about the axis of the open distal region to revolve within the aspiration zone.

Example 81: A method for removing clot from a blood vessel, the method comprising: introducing an aspiration catheter having a clot-extraction lumen into a blood vessel to position an open distal region of the clot-extraction lumen proximate clot; revolving a clot-disruption element about the open distal region to cause an open gap region between a periphery of the clot-disruption element and the open distal region to travel in an orbital path about the open distal region; and applying a negative pressure to a proximal end of the clot-extraction lumen to draw clot through the open gap region and into the clot-extraction lumen as the open distal gap region is travelling in the orbital path.

Example 82: A method according to Example 81, wherein the clot-disruption element is revolved within a rounded inner wall of the open distal region.

Example 83: A method according to Example 81 or 82, wherein revolving the clot-disruption element increases both a flow velocity and a dynamic pressure of the clot relative to the flow velocity and the dynamic pressure when the clot-disruption element is absent and the open distal region is unblocked.

Example 84: A method according to Example 83, wherein the flow velocity is increased by a factor of at least 1.5 and the dynamic pressure is increased by a factor of at least two.

Example 85: A method according to any of Examples 81-84, wherein the open gap region is diametrically opposed to a region on the periphery that lies closest to the inner wall of the open distal region.

Example 86: A method according to any of Examples 81-85, wherein the clot-disruption structure has a circular periphery.

Example 87: A method according to Example 86, wherein the circular periphery of the clot-disruption element has an outer diameter in a range from 25% to 95% of an inner diameter of the rounded inner wall of the open distal region.

Example 88: A method according to Example 86, wherein the circular periphery of the clot-disruption element has an outer diameter in a range from 35% to 85% of an inner diameter of the rounded inner wall open distal region.

Example 89: A method according to Example 86, wherein the circular periphery of the clot-disruption element has an outer diameter in a range from 50% to 75% of an inner diameter of the rounded inner wall open distal region.

Example 90: A method according to any of Examples 81-85, wherein the clot-disruption element has a non-circular periphery.

Example 91: A method according to any of Examples 81-90, wherein clot-disruption element is revolved in a plane transverse to a longitudinal axis of the open distal region.

Example 92: A method as in Example 91, wherein the plane is recessed behind the open distal region by a distance in a range from 0 to 10 mm.

Example 93: A method as in Example 92, wherein the plane is recessed behind the open distal region by a distance in a range from 0 to 5 mm.

Example 94: A method as in Example 92, wherein the plane is recessed behind the open distal region by a distance in a range from 0 to 1 mm.

Example 95: A method as in Example 92, wherein the plane is recessed behind the open distal region by a distance in a range from 0.1 to 1 mm.

Example 96: A method according to any of Examples 81-95, wherein the clot-disruption element comprises a substantially solid disc disposed in the open distal region and oriented in a plane normal to the central axis.

Example 97: A method according to Example 96, wherein substantially solid disc is free from gaps across its surface.

Example 98: A method according to Example 96 or 97, wherein the substantially solid disc has a generally circular periphery.

Example 99: A method according to Example 96 or 97, wherein the substantially solid disc has a non-circular geometry.

Example 100: A method according to any of Examples 81-99, wherein revolving the clot-disruption element comprises rotating a drive cable having a distal end coupled to an outer peripheral location on a proximal side of the clot-disrupting element.

Example 101: A method according to Example 100, wherein the proximal side of the clot-disruption element is attached to a retention ring which is rotatably mounted in an annular guide channel disposed on an inner wall of the open distal region while the clot-disruption element is being revolved.

Example 102: A method according to Example 101, wherein the drive cable is attached directly to the retention ring.

Example 103: A method according to Example 101, wherein the plate structure and the retention ring are joined by an axial strut.

Example 104: A method according to any of Examples 101-103, wherein the retention ring is positioned in the annular guide channel prior to introducing the aspiration catheter to the blood vessel.

Example 105: A method according to any of Examples 101-103, wherein the retention ring elastically contracts as it is advanced into the guide channel.

Example 106: A method according to any of Examples 101-103, wherein the retention ring has structure that inhibits accidental dislodgement from the annular guide channel.

Example 107: A method as in any of Examples 71-106, wherein revolving the clot-disruption structure comprises reversing the direction of revolution periodically to enhance aspiration.

Example 108: A method according to Example 107, wherein the direction of revolution is reversed every 0.1 sec to 10 sec.

Example 109: A system for removing clot from a blood vessel, the system comprising: a catheter body having a clot-extraction lumen extending from a proximal end to an open distal region, wherein the proximal end is configured to be attached to a vacuum source and the open distal region has a central axis therethrough; a plate structure disposed transversely across at least a portion of the open distal region, said plate structure configured to define a flow-blocked zone and an open aspiration zone though the open distal region; and a driver configured to revolve and/or rotate the plate structure about the central axis to cause the open aspiration zone to travel in an orbital path about the central axis of the open distal region; and wherein applying a negative pressure to the proximal end of the clot-extraction lumen draws a flow of clot through the open aspiration zone and into the clot-extraction lumen.

Example 110: A system according to Example 109, wherein the open distal region has a rounded inner wall.

Example 111: A system according to Example 109 or 110, wherein the plate structure is configured to generate a clot flow through the open aspiration zone having an increased flow velocity and an increased dynamic pressure relative to a flow velocity and a dynamic pressure of a clot flow into the open distal region in the absence of the plate structure.

Example 112: A system according to any of Examples 109-111, wherein the plate structure is asymmetrically positioned in the open distal region and is revolved about the central axis and wherein the aspiration zone is located between a peripheral edge of the plate structure and the rounded inner wall and follows an orbital path as the plate structure is revolved.

Example 113: A system according to Example 112, wherein the driver comprises a drive shaft having a distal end coupled to the plate structure at or near a peripheral edge thereof at a location diametrically opposed to the open aspiration zone, wherein rotation of the drive shaft causes a distal length of the drive shaft to precess about the central axis.

Example 114: A system according to any of Examples 109-111, wherein the plate structure is symmetrically positioned in the open distal region and is rotated about the central axis, wherein the aspiration zone comprises an off-axis, flow-restricting aperture through the plate structure.

Example 115: A system according to any of Examples 109-114, wherein the plate structure comprises a planar structure oriented in a plane normal to the central axis.

Example 116: A system according to any of Examples 109-115, wherein the open distal region has a cross-sectional area in a range from 1.2 mm² to 50 mm².

Example 117: A system according to Example 116, wherein the aspiration zone has a cross-sectional area in a range from 0.48 mm² to 40 mm².

Example 118: A system according to any of Examples 109-117, wherein the aspiration zone has a cross-sectional area in a range from 40% to 80% of a cross-sectional area of the open distal region.

Example 119: A system according to any of Examples 109-118, wherein the plate structure comprises a disc.

Example 120: A system according to Example 119, wherein the disc has an average thickness in a range from 0.1 mm to 1 mm.

Example 121: A system according to any of Examples 119-120, wherein the disc is planar on at least a distal surface.

Example 122: A system according to any of Examples 119-121, wherein the disc has a rounded periphery.

Example 123: A system according to Example 122, wherein the disc has a circular periphery.

Example 124: A system according to any of Examples 119-123, wherein the disc has a width which is less than an inner diameter of the open distal region and the aspiration zone is located between an outer periphery of the disc and an inner wall of the open distal region.

Example 125: A system according to Example 124, wherein the disc has an average width or diameter in a range from 25% to 95% of the inner diameter of the open distal region of the clot-extraction lumen.

Example 126: A system according to any of Examples 119-125, wherein the driver is configured to revolve the disc to cause the aspiration zone to revolve.

Example 127: A system according to any of Examples 119-123, wherein the disc has a width which is substantially equal to an inner diameter of the open distal region.

Example 128: A system according to Example 127, wherein the aspiration zone is located asymmetrically through the disc.

Example 129: A system according to Example 127, wherein the driver is configured to rotate the disc about the central axis of the open distal region, which causes the aspiration zone to revolve about the axis.

Example 130: A system according to any of Examples 119-129, wherein disc is substantially solid and free from gaps across its surface.

Example 131: A system according to any of Examples 119-130, wherein the substantially solid disc has a rounded peripheral edge.

Example 132: A system according to any of Examples 110-131, further comprising a circular retention ring coupled to and disposed proximally of the plate structure, wherein the retention ring is received in an annular guide channel disposed on an inner wall of the open distal region.

Example 133: A system according to Example 132, wherein the circular retention ring has an outer diameter approximately equal to an inner diameter of the open distal region of the clot-extraction lumen and comprises a distal bearing surface which engages a distal bearing surface on the guide channel.

Example 134: A system according to Example 133, wherein the circular retention ring further comprises a proximal bearing surface which engages a proximal bearing surface on the guide channel.

Example 135: A system according to Example 134, wherein the distal and proximal bearing surfaces on the annular guide channel are axially spaced apart to form an annular slot for rotationally receiving the retention ring.

Example 136: A system according to any of Examples 132-135, wherein the retention ring comprises a gap configured to allow the retention ring to elastically contract as the retention ring is inserted into the annular guide channel.

Example 137: A system according to any of Examples 132-136, wherein the plate structure and the retention ring are joined by an axial strut.

Example 138: A system according to Example 137, wherein a distal end of the axial strut is connected to a connection point on the periphery of the retention ring and has a dog leg configured to position the connection point immediately adjacent to the inner wall of the open distal region as the plate structure is revolved or rotated.

Example 139: A system according to any of Examples 110-138, wherein the driver comprises a drive cable and a motor configured to rotate the drive cable about an axis parallel to the central axis.

Example 140: A system according to Example 139, wherein the driver further comprises an outer sleeve having a longitudinal passage therethrough, wherein the outer sleeve is non-rotatably disposed over the drive cable.

Example 141: A system according to Example 140, wherein at least a portion of the drive cable is covered with a lubricous cover or coating.

Example 142: A system according to Example 140, wherein at least a portion of the sleeve is covered with a lubricous cover or coating.

Example 143: A system according to Example 140 or 141, wherein the driver comprises (1) a handle fixed to the drive cable and the outer sleeve and (2) a power module including the motor detachably secured to the handle, whereby the handle may be used to manipulate the drive shaft and sleeve in the clot-extraction lumen of the aspiration catheter prior to attaching the power module.

Example 144: A system according to Example 143, wherein the handle comprises an input drive connector connected to the drive cable.

Example 145: A system according to Example 144, wherein the handle further comprises a flush port fluidly connected to the outer sleeve.

Example 146: A system according to any of Examples 142-145, wherein the power module includes a battery, control circuitry, and an output drive connector connected to the motor.

Example 147: A system according to Example 146, wherein the input and output drive connectors comprise magnetic coupling elements.

Example 148: A system for removing clot from a blood vessel, comprising: a catheter body including (a) a proximal end and (b) a clot-extraction lumen having an open distal region having a diameter and a central axis therethrough; a clot-disruption structure having a distal clot-disruption element disposed proximate the open distal region of the clot-extraction lumen, said clot-disruption element having a width less than the diameter of the open distal region and being asymmetrically positioned thereacross; and a driver configured to rotate the clot disruption structure and revolve the clot-disruption element about the central axis of the open distal region of clot-extraction lumen as clot is aspirated into the open distal.

Example 149: A system according to Example 148, wherein the clot-disruption element comprises a planar structure oriented in a plane normal to the central axis.

Example 150: A system according to Example 149, wherein the planar structure comprises an open loop.

Example 151: A system according to Example 149, wherein the planar structure comprises a solid disc.

Example 152: A system according to any of Examples 149-151, wherein the clot-disruption element has an outer periphery which defines an asymmetric outer annular clot passage area disposed between the outer periphery and an inner wall of the open distal region.

Example 153: A system according to any of Examples 148-152, wherein the at least partially looped element comprises a continuous disruption ring structure.

Example 154: A system according to Example 153, wherein the continuous disruption ring structure has a circular geometry.

Example 155: A system according to Example 153, wherein the continuous disruption ring structure has a non-circular geometry.

Example 156: A system according to any of Examples 148-155, wherein the clot-disruption element has an average thickness in a range from 0.1 mm to 1 mm.

Example 157: A system according to Example 156, wherein the clot-disruption element has an average thickness in a range from 0.1 mm to 0.5 mm.

Example 158: A system according to any of Examples 148-155, wherein the clot-disruption element has an average width or diameter in a range from 25% to 95% of an inner diameter of the open distal region of the clot-extraction lumen.

Example 159: A system according to any of Examples 150-154, wherein the disruption ring structure has an outer diameter in a range from 25% to 95% of an inner diameter of the open distal region of the clot-extraction lumen, usually having an outer diameter in a range from 35% to 85% of an inner diameter of the open distal region of the clot-extraction lumen, sometimes having an outer diameter in a range from 50% to 75% of an inner diameter of the open distal region of the clot-extraction lumen.

Example 160: A system according to Example 148, wherein the clot-disruption element comprises a substantially solid disc disposed in the open distal region and oriented in a plane normal to the central axis.

Example 161: A system according to Example 160, wherein the substantially solid disc is free from gaps across its surface.

Example 162: A system according to Example 160 or 161, wherein the substantially solid disc has a generally circular periphery.

Example 163: A system according to Example 160 or 161, wherein the substantially solid disc has a non-circular periphery.

Example 164: A system according to any of Examples 160-163, wherein the substantially solid disc has an average thickness in a range from 0.1 mm to 1 mm.

Example 165: A system according to Example 164, wherein the substantially solid disc has an average thickness in a range from 0.1 mm to 0.5 mm.

Example 166: A system according to any of Examples 160-165, wherein the substantially solid disc has an outer diameter in a range from 25% to 95% of an inner diameter of the open distal region of the clot-extraction lumen, usually having an outer diameter in a range from 35% to 85% of an inner diameter of the open distal region of the clot-extraction lumen, sometimes having an outer diameter in a range from 50% to 75% of an inner diameter of the open distal region of the clot-extraction lumen.

Example 167: A system according to any of Examples 160-166, wherein the substantially solid disc has a rounded peripheral edge.

Example 168: A system according to any of Examples 149-167, wherein the clot-disruption structure further comprises a retention element disposed proximally of the clot-disruption element.

Example 169: A system according to Example 168, wherein the retention element comprises a circular retention ring having an outer diameter approximately equal to an inner diameter of the open distal region of the clot-extraction lumen and a distal bearing surface which engages a proximal bearing surface on a distal stop band disposed on an inner wall of the clot-extraction lumen in the distal open end.

Example 170: A system according to Example 168, wherein the circular retention ring on the clot-disruption structure has a proximal bearing surface which engages a distal bearing surface on a proximal stop band disposed on the inner wall of the clot-extraction lumen in the distal open end, wherein the distal and proximal stop bands are axially spaced apart to form an annular slot for rotationally receiving the retention ring of the clot-disruption structure.

Example 171: A system according to Example 169 or 170, wherein the retention ring comprises a continuous circle.

Example 172: A system according to Example 170, wherein the retention ring comprises a gap configured to allow the retention ring to elastically contract as the retention ring is moved over the proximal stop band.

Example 173: A system according to any of Examples 170-172, wherein the proximal stop band has a smaller opening than the distal stop band.

Example 174: A system according to any of Examples 169-173, wherein the clot-disruption element and the retention ring are joined by an axial strut.

Example 175: A system according to Example 174, wherein a distal end of the axial strut is connected to a connection point on the periphery of the retention ring and has a dog leg configured to position the connection point immediately adjacent to an inner surface of the open distal region of the catheter body as the clot-disruption structure is revolved.

Example 176: A system according to any of Examples 168-170, wherein the retention ring is disposed proximally of the clot-disruption element by an offset distance in a range from 0 mm to 15 mm, often from 0.5 mm to 15 mm, typically from 0.5 mm to 10 mm, with preferred offset distances in ranges of 0.5 mm to 1 mm and 1.5 mm to 2.5 mm.

Example 177: A system according to Example 169, wherein the retention ring is joined directly to the clot-disruption element with no axial offset.

Example 178: A system according to any of Examples 169-177, wherein the distal bearing surface of the retention ring and the proximal bearing surface of the distal stop band each have an annular geometry, wherein the annular bearing surfaces are brought together when the clot-disruption structure is fully advanced in the distal direction in the clot-extraction lumen.

Example 179: A system according to any of Examples 148-178, wherein the driver comprises a drive cable and a motor configured to rotate the drive cable about an axis parallel to the central axis.

Example 180: A system according to Example 179, wherein a distal end of the drive cable has a fixed point of attachment proximate a peripheral edge of the clot-disruption structure, wherein the fixed point of attachment is radially offset from the central axis of the open distal region of clot-extraction lumen so that the clot-disruption structure simultaneously revolves about the central axis and rotates about a center point of the clot-disruption element as the drive cable is rotated.

Example 181: A system according to Example 180, wherein the driver further comprises an outer sleeve having a longitudinal passage therethrough, wherein the outer sleeve is non-rotatably disposed in the clot-extraction lumen and the drive cable is rotatably mounted in the longitudinal passage.

Example 182: A system according to any of Examples 179-181, wherein at least a portion of the drive cable is covered with a lubricous cover or coating.

Example 183: A system according to any of Examples 179-182, wherein the driver is further configured to apply a distal biasing force on the drive cable to maintain forward positioning of the clot-disruption structure.

Example 184: A system according to any of Examples 179-182, wherein the retention ring is axially constrained to maintain positioning of the clot-disruption structure in the absence of biasing force provided by the drive cable.

Example 185: A system according to any of Examples 179-182, wherein the retention ring is axially constrained to maintain positioning of the clot-disruption structure in the presence of axial load exerted by the drive cable.

Example 186: A system according to any of Examples 179-182, further comprising a detachable handle comprising the motor, wherein the handle is configured to rotationally drive the driver and non-rotationally secure the catheter body.

Example 187: A system according to Example 186, wherein the handle comprises a flush port.

Example 188: A system according to Example 185 or 187, wherein the handle comprises a battery.

Example 189: A system according to any of Examples 177-188, wherein the handle comprises control circuitry.

Example 190: A system according to any of Examples 185-189, wherein the handle comprises a distal housing detachably connectable to a proximal housing.

Example 191: A system according to Example 190, wherein the distal housing comprises a flush port and a proximal drive connector.

Example 192: A system according to Example 191, wherein the proximal housing comprises the motor, control circuitry, batteries, and a distal drive connector.

Example 193: A system according to Example 192, wherein the proximal and distal drive connectors comprise magnetic coupling elements.

Example 194: A method of removing a clot material, the method comprising: positioning an aspiration catheter so that an opening into an aspiration lumen of the aspiration catheter is proximate to a clot material; moving a disruptor in a plane that is parallel to the opening of the aspiration catheter in an orbital manner so that an opening into the aspiration lumen between the disruptor and an inner wall of the aspiration opening moves around within the aspiration opening; and applying suction while moving the disruptor to draw the clot material through the moving opening into the aspiration lumen between the disruptor and an inner wall of the aspiration opening.

Example 195: A method according to Example 194, wherein moving the disruptor comprises moving a disc-shaped disruptor.

Example 196: A method according to Example 194 or 195, further comprising positioning the disruptor in the plane by inserting the disruptor through the lumen of the aspiration catheter prior to moving the disruptor in the plane that is parallel to the aspiration opening.

Example 197: A method according to any of Examples 194-196, wherein moving the disruptor in the plane comprises rotating a drive cable that is eccentrically coupled to the disruptor.

Example 198: A method according to any of Examples 194-197, wherein the opening into the aspiration lumen comprises a crescent-shaped opening.

Example 199: A method according to any of Examples 194-198, wherein suction is started before moving the disruptor.

Example 200: A method according to any of Examples 194-198, wherein suction is started after moving the disruptor.

Example 201: A method according to any of Examples 194-200, further comprising contacting the clot material with a distal face of the disruptor prior to moving the disruptor in the plane that is parallel to the opening of the aspiration catheter.

Example 202: A method according to any of Examples 194-201, further comprising maintaining the disruptor at or just proximal to the opening of the aspiration catheter.

Example 203: A method of removing a clot material, the method comprising: positioning an aspiration catheter comprising an aspiration lumen so that an opening into the aspiration lumen is proximate to a clot material; rotating a drive shaft to eccentrically revolve a disruptor in a plane that is parallel to the opening of the aspiration catheter in an orbital manner so that an opening into the aspiration lumen between the disruptor and an inner wall of the aspiration opening moves around within the aspiration opening; and applying suction while moving the disruptor to draw the clot material through the moving opening into the aspiration lumen between the disruptor and an inner wall of the aspiration opening.

Example 204: A method according to Example 203, wherein moving the disruptor comprises moving a disc-shaped disruptor.

Example 205: A method according to Example 203 or 204, further comprising positioning the disruptor in the plane by inserting the disruptor through the lumen of the aspiration catheter prior to moving the disruptor in the plane that is parallel to the aspiration opening.

Example 206: A method according to any of Examples 203-205, wherein the opening into the aspiration lumen comprises a crescent-shaped opening.

Example 207: A method according to any of Examples 203-206, wherein suction is started before moving the disruptor.

Example 208: A method according to any of Examples 203-207, wherein suction is started after moving the disruptor.

Example 209: A method according to any of Examples 203-208, further comprising contacting the clot material with a distal face of the disruptor prior to moving the disruptor in the plane that is parallel to the opening of the aspiration catheter.

Example 210: A method according to any of Examples 203-209, further comprising maintaining the disruptor at or just proximal to the opening of the aspiration catheter.

Example 211: A method of removing a clot material, the method comprising: positioning an aspiration catheter comprising an aspiration lumen so that an opening into the aspiration lumen is proximate to a clot material; revolving a disruptor in a plane that is parallel to the opening of the aspiration catheter in an eccentric manner so that an opening into the aspiration lumen between an outer edge of the disruptor and an inner wall of the aspiration opening revolves around the aspiration opening; and applying suction while moving the disruptor to draw clot material through the moving opening into the aspiration lumen.

Example 212: A method, the method comprising: positioning an aspiration catheter so that an opening into an aspiration lumen through the aspiration catheter is proximate to a clot material; and eccentrically revolving a disc-shaped disruptor transversely around an opening into the aspiration lumen so that a distal face of the disc-shaped disruptor moves orbitally across the opening, while applying suction to draw clot material past the orbitally moving distal face and through the opening.

Example 213: A method, the method comprising: positioning an aspiration catheter so that an opening into an aspiration lumen of the aspiration catheter is proximate to a clot material; and dynamically changing the shape of the opening into the aspiration lumen by eccentrically revolving a disruptor transversely across the passage; and applying suction to draw clot material through the opening and into the aspiration lumen.

Example 214: A method, the method comprising: positioning an aspiration catheter so that an opening into an aspiration lumen of the aspiration catheter is proximate to a clot material; and eccentrically revolving a disc-shaped disruptor transversely around an opening into the aspiration lumen by continuously accelerating, decelerating and reversing the rotation of a drive shaft coupled to the disc-shaped disruptor; and applying suction to draw the clot material against the disc-shaped disruptor and into the aspiration lumen.

Example 215: A method, the method comprising: positioning a catheter within a vessel; advancing a disruptor assembly within a lumen of the catheter until the disruptor assembly engages an annular guide assembly within a distal end region of the lumen so that a disruptor at a distal end of the disruptor assembly is positioned eccentrically across an opening into the lumen of the catheter to occlude a portion of the opening that is offset from a center of the opening; and rotating a drive shaft to eccentrically revolve the disruptor transversely around the opening while applying aspiration through the lumen.

Example 216: A method according to Example 215, further comprising advancing the catheter against a clot material to aspirate the clot material into the lumen of the catheter while rotating the drive shaft to dynamically compress the clot material between an edge of the revolving disruptor and an inner diameter of the opening.

Example 217: A method according to Example 215 or 216, further comprising advancing the catheter against a clot material to alter the viscosity of the clot material and aspirate the clot material into the lumen of the catheter.

Example 218: A method according to any of Examples 215-217, wherein rotating the drive shaft comprises rotating the drive shaft within a sleeve extending through the lumen of the catheter.

Example 219: A method according to any of Examples 215-218, wherein the disruptor assembly engages the annular guide assembly by engaging a retention protrusion of the disruptor assembly within the annular guide assembly.

Example 220: A method according to any of Examples 215-219, wherein the annular guide assembly is between 1 mm and 5 cm of a distal end of the lumen.

Example 221: A method according to any of Examples 215-220, wherein the disruptor assembly is positioned eccentrically across the opening into the lumen of the catheter to occlude between 30-95% of the opening.

Example 222: A method according to any of Examples 215-221, wherein rotating the drive shaft comprises rotating the drive shaft between about 500 and 7000 RPM.

Example 223: A method according to any of Examples 215-222, wherein rotating the drive shaft comprises alternating the direction of rotation.

Example 224: A method according to any of Examples 215-223, further comprising advancing the catheter, with the disruptor positioned eccentrically across the opening, against a clot material.

Example 225: A method, the method comprising: positioning a catheter within a vessel; advancing a disruptor assembly within a lumen of the catheter until a retention protrusion of the disruptor assembly engages an annular guide assembly that is between 1 mm and 5 cm of a distal end of the lumen so that a disruptor at a distal end of the disruptor assembly is positioned eccentrically across an opening into the lumen of the catheter to occlude a portion of the opening; and rotating a drive shaft to eccentrically revolve the disruptor transversely around the opening while retaining the retention protrusion within the annular guide assembly; and applying aspiration through the lumen.

Example 226: A method according to Example 225, further comprising advancing the catheter against a clot material to aspirate the clot material into the lumen of the catheter while rotating the drive shaft to dynamically compress the clot material between an edge of the revolving disruptor and an inner diameter of the opening.

Example 227: A method according to Example 225 or 226, further comprising advancing the catheter against a clot material to alter the viscosity of the clot material and aspirate the clot material into the lumen of the catheter.

Example 228: A method according to any of Examples 225-227, wherein rotating the drive shaft comprises rotating the drive shaft within a sleeve extending through the lumen of the catheter.

Example 229: A method according to any of Examples 225-228, wherein engaging the retention protrusion within the annular guide assembly comprises engaging one or more of: a retention ring forming the retention protrusion, a split ring forming the retention protrusion, one or more wings forming the retention protrusion.

Example 230: A method according to any of Examples 225-229, wherein engaging the retention protrusion within the annular guide assembly comprises engaging the retention protrusion against or between one or more of: a ring forming the annular guide assembly, a pair of rings forming the annular guide assembly, a channel forming the annular guide assembly.

Example 231: A method according to any of Examples 225-230, wherein the disruptor assembly is positioned eccentrically across the opening into the lumen of the catheter to occlude between 30-95% of the opening.

Example 232: A method according to any of Examples 225-231, wherein rotating the drive shaft comprises rotating the drive shaft between about 500 and 7000 RPM.

Example 233: A method according to any of Examples 225-232, wherein rotating the drive shaft comprises alternating the direction of rotation.

Example 234: A method, the method comprising: positioning a catheter within a vessel; advancing a disruptor assembly within a lumen of the catheter until a retention protrusion of the disruptor assembly engages an annular guide assembly within a distal end region of the lumen so that a disruptor at a distal end of the disruptor assembly is positioned eccentrically across an opening into the lumen of the catheter to occlude between 30-95% of the opening; and rotating a drive shaft to eccentrically revolve the disruptor transversely around the opening while retaining the retention protrusion within the annular guide assembly; and applying aspiration through the lumen.

Example 235: A system, the system comprising: a catheter having a lumen extending therethrough, an opening into the lumen, and an annular guide assembly within a distal end region of the lumen; and a disruptor assembly configured to be positioned within the catheter, the disruptor assembly comprising: a disruptor at a distal end of the disruptor assembly having a distal face that is configured to partially occlude the opening into the lumen of the catheter; a retention protrusion proximal to the disruptor and configured to movably engage the annular guide assembly of the catheter to limit axial movement of the disruptor assembly in the lumen; and an elongated drive cable eccentrically coupled to the disruptor and configured to be rotated to drive revolution of the disruptor across the opening into the lumen of the catheter when the retention protrusion is engaged with the annular guide assembly of the catheter.

Example 236: A system according to Example 235, wherein the annular guide assembly comprises one or more annular rings.

Example 237: A system according to Example 235 or 236, wherein the annular guide assembly is between 2 mm and 5 cm from a distal end of the catheter.

Example 238: A system according to any of Examples 235-237, wherein the disruptor is coupled to the retention protrusion by a dog leg member.

Example 239: A system according to any of Examples 235-238, wherein the distal face of the disruptor is configured to occlude between 30%-95% of the opening into the lumen of the catheter when the retention protrusion is engaged with the annular guide assembly.

Example 240: A system according to any of Examples 235-239, wherein the disruptor comprises a disc-shaped disruptor.

Example 241: A system according to any of Examples 235-240, wherein the distal face of the disruptor comprises a convex surface.

Example 242: A system according to any of Examples 235-241, wherein the distal face of the disruptor comprises a substantially flat surface.

Example 243: A system according to any of Examples 235-242, wherein the retention protrusion is positioned between 1 mm and 5 cm from the disruptor.

Example 244: A system according to any of Examples 235-243, wherein the retention protrusion comprises one or more of: a retention ring, a one or more wings or arms, a split ring.

Example 245: A system according to any of Examples 235-244, further comprising a sleeve extending over at least a proximal length of the drive cable, wherein the drive cable is configured to rotate relative to the sleeve.

Example 246: A system according to any of Examples 235-245, further comprising a proximal drive connector at a proximal end of the drive cable configured to connect to a rotary driver.

Example 247: A system according to any of Examples 235-246, wherein the retention protrusion of the disruptor assembly is removably coupled to the annular guide assembly.

Example 248: A system, the system comprising: a catheter having a lumen extending therethrough, an opening into the lumen, and an annular guide assembly within a distal end region of the lumen; and a disruptor assembly configured to be positioned within the catheter, the disruptor assembly comprising: a disruptor at a distal end of the disruptor assembly having a distal face that is configured to occlude between 30%-95% of the opening into the lumen of the catheter; a retention protrusion proximal to be positioned between 1 mm and 5 cm from the disruptor and configured to slidably engage with the annular guide assembly of the catheter; and an elongate drive cable eccentrically coupled to the disruptor and configured to be rotated to drive revolution of the disruptor orbitally within the opening into the lumen of the catheter when the retention protrusion is engaged with the annular guide assembly of the catheter.

Example 249: A system according to Example 248, wherein the annular guide assembly comprises one or more annular rings.

Example 250: A system according to Example 248 or 249, wherein the annular guide assembly is between 2 mm and 5 cm from a distal end of the catheter.

Example 251: A system according to Example 248-250, wherein the disruptor comprises a disc-shaped disruptor.

Example 252: A system according to Example 248-251, wherein the distal face of the disruptor comprises a convex surface.

Example 253: A system according to Example 248-252, wherein the retention protrusion comprises one or more of: a retention ring, a one or more wings or arms, a split ring.

Example 254: A system according to Example 248-253, further comprising a sleeve extending over at least a proximal length of the drive cable, wherein the drive cable is configured to rotate relative to the sleeve.

Example 255: A system according to Example 248-254, further comprising a proximal drive connector at a distal end of the drive cable configured to couple to a rotary driver.

Example 256: A system, the system comprising: a catheter having a lumen extending therethrough, an opening into the lumen, and an annular guide assembly within a distal end region of the lumen; a disruptor assembly configured to be positioned within the catheter, the disruptor assembly comprising: a disruptor at a distal end of the disruptor assembly having a disc-shaped distal face configured to partially occlude the opening into the lumen; a retention protrusion positioned between 1 mm and 5 cm from the disruptor and configured to engage with the annular guide assembly within the lumen of the catheter; and an elongate drive cable eccentrically coupled to the disruptor and configured to be rotated to drive revolution of the disruptor eccentrically about the opening into the lumen when the retention protrusion is engaged with the annular guide assembly of the catheter.

Example 257: A disruptor assembly apparatus configured to be positioned within an aspiration catheter to remove clot, the apparatus comprising: a disruptor at a distal end of the disruptor assembly having a distal face that is configured to partially occlude an opening into a lumen of the catheter; a retention protrusion proximal to the disruptor and configured to movably engage an annular guide assembly within the catheter lumen to limit axial movement of the disruptor assembly in the lumen; and an elongated drive cable eccentrically coupled to the disruptor and configured to be rotated to drive revolution of the disruptor across the opening into the lumen of the catheter when the retention protrusion is engaged with the annular guide assembly of the catheter.

Example 258: A system according to Example 257, wherein the disruptor is coupled to the retention protrusion by a dog leg member.

Example 259: A system according to any of Examples 257-258, wherein the distal face of the disruptor is configured to occlude between 30%-95% of the opening into the lumen of the catheter when the retention protrusion is engaged with the annular guide assembly.

Example 260: A system according to any of Examples 257-259, wherein the disruptor comprises a disc-shaped disruptor.

Example 261: An apparatus according to any of Examples 257-260, wherein the distal face of the disruptor comprises a convex surface.

Example 262: An apparatus according to any of Examples 257-261, wherein the distal face of the disruptor comprises a substantially flat surface.

Example 263: An apparatus according to any of Examples 257-262, wherein the retention protrusion is positioned between 1 mm and 5 cm from the disruptor.

Example 264: An apparatus according to any of Examples 257-263, wherein the retention protrusion comprises one or more of: a retention ring, one or more wings or arms, a split ring.

Example 265: An apparatus according to any of Examples 257-264, further comprising a sleeve extending over at least a proximal length of the drive cable, wherein the drive cable is configured to rotate relative to the sleeve.

Example 266: An apparatus according to any of Examples 257-265, further comprising a proximal drive connector at a distal end of the drive cable configured to connect to a rotary driver.

Example 267: A disruptor assembly apparatus configured to be positioned within an aspiration catheter to remove clot, the apparatus comprising: a disc-shaped disruptor at a distal end of the disruptor assembly having a distal face that is configured to partially occlude an opening into a lumen of the catheter; a retention protrusion proximal to the disruptor and configured to movably engage an annular guide assembly within the catheter lumen to limit axial movement of the disruptor assembly in the lumen; a connector connecting the disc-shaped disruptor to the retention protrusion so that the distal face is perpendicular to a long axis of the connector; and an elongated drive cable eccentrically coupled to the disruptor through the connector wherein the elongated drive cable is configured to be rotated to drive revolution of the disruptor across the opening into the lumen of the catheter when the retention protrusion is engaged with the annular guide assembly of the catheter.

Example 268: A disruptor assembly apparatus according to Example 267, wherein the connector is a dog leg member.

Example 269: A disruptor assembly apparatus according to Example 267 or 268, wherein the distal face of the disruptor is configured to occlude between 30%-95% of the opening into the lumen of the catheter when the retention protrusion is engaged with the annular guide assembly.

Example 270: A disruptor assembly apparatus according to any of Examples 267-269, wherein the distal face of the disruptor comprises a convex surface.

Example 271: A disruptor assembly apparatus according to any of Examples 267-270, wherein the distal face of the disruptor comprises a substantially flat surface.

Example 272: A disruptor assembly apparatus according to any of Examples 267-271, wherein the retention protrusion is positioned between 1 mm and 5 cm from the disruptor.

Example 273: A disruptor assembly apparatus according to any of Examples 267-272, wherein the retention protrusion comprises one or more of: a retention ring, one or more wings or arms, a split ring.

Example 274: A disruptor assembly apparatus according to any of Examples 267-273, further comprising a sleeve extending over at least a proximal length of the drive cable, wherein the drive cable is configured to rotate relative to the sleeve.

Example 275: A disruptor assembly apparatus according to any of Examples 267-274, further comprising a proximal drive connector at a distal end of the drive cable configured to connect to a rotary driver.

Example 276: A disruptor assembly apparatus configured to be positioned within an aspiration catheter to remove clot, the apparatus comprising: a disc-shaped disruptor at a distal end of the disruptor assembly having a distal face that is configured to partially occlude an opening into a lumen of the catheter; a retention protrusion proximal to the disruptor and configured to movably engage an annular guide assembly within the catheter lumen to limit axial movement of the disruptor assembly in the lumen; a connector connecting the disc-shaped disruptor to the retention protrusion so that the distal face is perpendicular to a long axis of the connector, wherein the connector extends between 2 mm and 5 cm; an elongated drive cable eccentrically coupled to the disruptor through the connector wherein the elongated drive cable is configured to be rotated to drive revolution of the disruptor across the opening into the lumen of the catheter when the retention protrusion is engaged with the annular guide assembly of the catheter; and a sleeve extending over at least a proximal length of the drive cable, wherein the drive cable is configured to rotate relative to the sleeve.

Example 277: A hand-held rotary driver device, the device comprising: a drive motor; a housing enclosing the drive motor; a drive shaft coupled to the drive motor and extending distally; a distal drive connector on a distal end of the drive shaft, wherein the distal drive connector is configured to engage a proximal drive connector of a disruptor assembly apparatus; and a controller coupled to the drive motor and configured to drive rotation of the drive shaft at between 500 revolutions per minute (RPM) and 7000 RPM, wherein the controller is configured to alternate rotation of the drive shaft clockwise and counterclockwise.

Example 278: A device according to Example 277, wherein the controller is configured to continuously accelerate and decelerate the rotation of the drive shaft.

Example 279: A device according to Example 277, wherein the controller is configured to cause the drive shaft to alternately rotate clockwise for a first duration, pause, and rotate counterclockwise for a second duration.

Example 280: A device according to Example 279, wherein the first duration, pause and second duration are each between about 0.1 second and 10 seconds.

Example 281: A device according to any of Examples 277-280, wherein the distal drive connector is configured to engage the proximal drive connector of a disruptor assembly apparatus through a keyed interface.

Example 282: A device according to any of Examples 277-281, wherein the distal drive connector comprises one or more magnets to engage the proximal drive connector of a disruptor assembly apparatus.

Example 283: A device according to any of Examples 277-282, further comprising an audible indicator within the housing that is configured to emit a sound indicating operation of the drive motor.

Example 284: A device according to Example 283, wherein the audible indicator comprises a clacker configured to emit a sound indicating a change in rotational direction of the drive motor.

Example 285: A device according to any of Examples 277-284, wherein the housing is configured as a handle.

Example 286: A device according to any of Examples 277-285, further comprising a control to activate driving of the drive shaft.

Example 287: A hand-held rotary driver device, the device comprising: a drive motor; a housing enclosing the drive motor, wherein the housing is configured as a handle; a drive shaft coupled to the drive motor and extending distally; a distal drive connector on a distal end of the drive shaft, wherein the distal drive connector comprises a mechanical interlock configured to engage a proximal drive connector of a disruptor assembly apparatus; and a controller coupled to the drive motor and configured to drive rotation of the drive shaft at between 500 revolutions per minute (RPM) and 7000 RPM, wherein the controller is configured to alternate rotation of the drive shaft clockwise and counterclockwise and to continuously accelerate and decelerate the rotation of a drive shaft.

Example 288: A device according to Example 287, wherein the mechanical interlock comprises one or more keyed projections configured to engage with a corresponding one or more recesses on the proximal drive connector.

Example 289: A device according to Example 287 or 288, wherein the controller is configured to cause the drive shaft to alternately rotate clockwise for a first duration, pause for a second duration, and rotate counterclockwise for a third duration.

Example 290: A device according to Example 289, wherein the first duration, second duration and third duration are each between about 0.1 second and 10 seconds.

Example 291: A device according to Example 289 or 290, wherein at least two of the first duration, the second duration and the third duration are different lengths of time.

Example 292: A device according to any of Examples 287-291, wherein the drive shaft connector comprises one or more magnets to engage the proximal drive connector of a disruptor assembly apparatus.

Example 293: A device according to any of Examples 287-292, further comprising an audible indicator within the housing that is configured to emit a sound indicating operation of the drive motor.

Example 294: A device according to Example 293, wherein the audible indicator comprises a clacker configured to emit a sound indicating a change in rotational direction of the drive motor.

Example 295: A device according to any of Examples 287-294, further comprising a control to activate driving of the drive shaft.

Example 296: A hand-held rotary driver device, the device comprising: a drive motor; a housing enclosing the drive motor, wherein the housing is configured as a handle; a drive shaft coupled to the drive motor and extending distally; a distal drive connector on a distal end of the drive shaft, wherein the distal drive connector comprises a mechanical interlock configured to engage a proximal drive connector of a disruptor assembly apparatus; and a controller coupled to the drive motor and configured to drive rotation of the drive shaft at between 500 revolutions per minute (RPM) and 7000 RPM, wherein the controller is configured to cause the drive shaft to alternately rotate clockwise for a first duration, pause, and rotate counterclockwise for a second duration, wherein the first duration, pause and second duration are each between about 0.1 second and 10 seconds.

Example 297: A system for removing clot from a blood vessel, the system comprising: an aspiration catheter body having a clot-extraction lumen extending from a proximal end to an open distal region; a clot disruption element disposed transversely across at least a portion of the open distal region, said clot disruption element having an axis and being a disc structure configured to define a flow-restricting zone and an open aspiration zone through the open distal region; and a driver configured to drive the clot disruption element to cause the clot disruption element to revolve about its axis and the open aspiration zone to travel in an orbital path about the central axis of the open distal region; wherein the proximal end is configured to be coupled to a vacuum source to apply a negative pressure to the proximal end of the clot-extraction lumen to draw a flow of clot through the open aspiration zone and into the clot-extraction lumen.

Although certain embodiments or examples of the disclosure have been described in detail, variations and modifications will be apparent to those skilled in the art, including embodiments or examples that may not provide all the features and benefits described herein. It will be understood by those skilled in the art that the present disclosure extends beyond the specifically disclosed embodiments or examples to other alternative or additional examples or embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub combinations of the specific features of the embodiments and examples may be made and still fall within the scope of the present disclosure. Accordingly, it should be understood that various features of the disclosed embodiments can be combined with or substituted for one another in order to form varying modes or examples of the present disclosure. Thus, it is intended that the scope of the present disclosure herein disclosed should not be limited by the particular disclosed embodiments or examples described above. For all of the embodiments and examples described above, the steps of any methods for example need not be performed sequentially.

What is claimed is:

1. A system for removing clot from a blood vessel, said system comprising:
an aspiration catheter body having a clot-extraction lumen extending from a proximal end to an open distal region, wherein the proximal end is configured to be coupled to a vacuum source and the open distal region has a central axis therethrough;
a substantially solid disc structure disposed transversely across at least a portion of the open distal region, said disc structure configured to define a flow-blocked zone and an open aspiration zone through the open distal region; and
a driver configured to rotate and revolve the disc structure about the central axis to cause the open aspiration zone to travel in an orbital path about the central axis of the open distal region;
wherein applying a negative pressure to the proximal end of the clot-extraction lumen draws a flow of clot through the open aspiration zone and into the clot-extraction lumen.

2. The system of claim 1, wherein the disc structure is configured to generate a clot flow through the open aspiration zone having an increased flow velocity and an increased dynamic pressure relative to a flow velocity and a dynamic pressure of a clot flow into the open distal region in the absence of the disc structure.

3. The system of claim 1, wherein the disc structure is asymmetrically positioned in the open distal region and is revolved about the central axis and wherein the aspiration zone is located between a peripheral edge of the disc structure and a rounded inner wall of the open distal region and follows an orbital path as the disc structure is revolved.

4. The system of claim 1, wherein the driver comprises a drive shaft having a distal end coupled to the disc structure at or near a peripheral edge thereof at a location diametrically opposed to the open aspiration zone, wherein rotation of the drive shaft causes a distal length of the drive shaft to precess about the central axis.

5. The system of claim 1, wherein the disc structure comprises a planar structure oriented in a plane normal to the central axis.

6. The system of claim 1, wherein the open distal region has a rounded inner wall and a cross-sectional area in a range from 1.2 mm² to 50 mm² and the aspiration zone has a cross-sectional area in a range from 0.48 mm² to 40 mm².

7. The system of claim 1, wherein the aspiration zone has a cross-sectional area in a range from 40% to 80% of a cross-sectional area of the open distal region.

8. The system of claim 1, wherein the disc structure is planar on at least a distal surface and has an average thickness in a range from 0.1 mm to 1 mm.

9. The system of claim 8, wherein the disc structure has an average width or diameter in a range from 25% to 95% of an inner diameter of the open distal region of the clot-extraction lumen.

10. The system of claim 8, wherein the disc structure is substantially flush with the open distal region of the clot-extraction lumen.

11. The system of claim 1, wherein the driver comprises a drive shaft and a motor configured to rotate the drive shaft about an axis parallel to the central axis.

12. The system of claim 11, wherein the driver further comprises an outer sleeve having a longitudinal passage therethrough, wherein the outer sleeve is non-rotatably disposed over the drive shaft.

13. The system of claim 12, wherein at least a portion of the drive shaft or sleeve is covered with a lubricous cover or coating.

14. The system of claim 12, wherein the driver comprises (1) a handle fixed to the drive shaft and the outer sleeve and (2) a power module including the motor detachably secured to the handle, whereby the handle may be used to manipulate the drive shaft and sleeve in the clot-extraction lumen of the aspiration catheter prior to attaching the power module.

15. The system of claim 14, wherein the handle comprises an input drive connector connected to the drive shaft and the power module includes a battery, control circuitry, and an output drive connector connected to the motor, wherein the input and output drive connectors comprise magnetic coupling elements.

16. A method for removing clot from a blood vessel, said method comprising:
introducing an aspiration catheter into a blood vessel to position an open distal region of the aspiration catheter proximate clot;
rotating and revolving a substantially solid disc structure defining a flow-blocking zone about a central axis of the open distal region while leaving an open aspiration zone around the disc structure; and
applying a negative pressure to a clot-extraction lumen to draw clot through the open aspiration zone and into the clot-extraction lumen to increase both a flow velocity and a dynamic pressure of the clot relative to the flow velocity and the dynamic pressure when the disc structure is absent.

17. The method of claim 16, further comprising positioning the flow-blocking disc structure transversely across the open distal region.

18. The method of claim 17, wherein the flow-blocking disc structure is positioned across the open distal region before introducing the aspiration catheter into the blood vessel.

19. The method of claim 16, wherein the flow velocity is increased by a factor of at least 1.5 and the dynamic pressure is increased by a factor of at least two.

20. The method of claim 16, further comprising rotating and revolving the disc structure to revolve the open aspiration zone about an axis of the open distal region.

21. The method of claim 16, wherein the open distal region has a cross-sectional area in a range from 1.2 mm² to 50 mm² and the aspiration zone has a cross-sectional area in a range from 0.48 mm² to 40 mm².

22. The method of claim 16, wherein the aspiration zone has a cross-sectional area in a range from 40% to 80% of a cross-sectional area of the distal region.

23. The method of claim 16, wherein the disc structure is planar on at least a distal surface and has an average thickness in a range from 0.1 mm to 1 mm.

24. The method of claim 16, wherein the disc structure has a width which is less than an inner diameter of the open distal region and the aspiration zone is located between an outer periphery of the disc structure and an inner wall of the open distal region.

25. The method of claim 16, wherein the aspiration catheter and flow-blocking disc structure are positioned in a middle cerebral artery for the treatment of ischemic stroke.

26. The method of claim 25, wherein the negative pressure is applied to the clot-extraction lumen for a period of time in a range from 5 seconds to 30 seconds for substantial removal of the clot from the middle cerebral artery in a single pass treatment.

27. The method of claim 16, wherein the aspiration catheter and flow-blocking disc structure are positioned in an internal carotid artery for the treatment of ischemic stroke.

28. The method of claim 27, wherein the negative pressure is applied to the clot-extraction lumen for a period of time in a range from 5 seconds to 60 seconds for substantial removal of the clot from the internal carotid artery in a single pass treatment.

29. The method of claim 16, wherein the disc structure is coupled to a drive shaft, further comprising rotating the drive shaft around 4000 revolutions per minute.

30. The method of claim 29, further comprising reversing a direction of the revolutions every 0.5 seconds with a 0.1 millisecond pause therebetween.
